SEQUENCE LISTING (64 sequences)

SEQ ID NO: 1 - protein, 34 aa, Homo sapiens
CKKHELYVSFRDLGWQDWIIAPEGYAAYYCEGEC

SEQ ID NO: 2 - protein, 33 aa, Homo sapiens
AFPLNSYMNATNHAIVQTLVHFINPETVPKPCC

SEQ ID NO: 3 - protein, 34 aa, Homo sapiens
APTQLNAISVLYFDDSSNVILKKYRNMVVRACGC

SEQ ID NO: 4 - protein, 102 aa, Homo sapiens
CKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 5 - protein, 26 aa, Homo sapiens
CELYVSFRDLGWQDWIIAPEGYAAYC

SEQ ID NO: 6 - protein, 15 aa, Homo sapiens
CFRDLGWQDWIIAPC

SEQ ID NO: 7 - protein, 11 aa, Homo sapiens
ECRDLGWQDWC

SEQ ID NO: 8 - protein, 11 aa, Homo sapiens
CWDQWGLDRCE

SEQ ID NO: 9 - protein, 14 aa, Homo sapiens
CRDLGWQDWIIAPC

SEQ ID NO: 10 - protein, 14 aa, Homo sapiens
CPAIIWDQWGLDRC

SEQ ID NO: 11 - protein, 32 aa, Homo sapiens
CAFPLNSYMNATNHAIVQTLVHFINPETVPKC

SEQ ID NO: 12 - protein, 13 aa, Homo sapiens
CLNSYMNATNHAC

SEQ ID NO: 13 - protein, 11 aa, Homo sapiens
CCFINPETVCC

SEQ ID NO: 14 - protein, 9 aa, Homo sapiens
CFINPETVC

SEQ ID NO: 15 - protein, 9 aa, Homo sapiens
CVTEPNIFC

SEQ ID NO: 16 - protein, 28 aa, Homo sapiens
CLNAISVLYFDDSSNVILKKYRNMVVRC

SEQ ID NO: 17 - protein, 11 aa, Homo sapiens
CYFDDSSNVIC

SEQ ID NO: 18 - protein, 11 aa, Homo sapiens
CIVNSSDDFYC

SEQ ID NO: 19 - protein, 16 aa, Homo sapiens
CYFDDSSNVICKKYRS

SEQ ID NO: 20 - protein, 16 aa, Homo sapiens
SRYKKCIVNSSDDFYC

SEQ ID NO: 21 - protein, 15 aa, Homo sapiens
CFDDSSNVCLKKYRS

SEQ ID NO: 22 - protein, 15 aa, Homo sapiens
CFDDSSNVICKKYRS

SEQ ID NO: 23 - protein, 16 aa, Homo sapiens
CYFDDSSNVCLKKYRS

SEQ ID NO: 24 - protein, 20 aa, Homo sapiens
CYFDDSSNVICKKYRGSGSC

SEQ ID NO: 25 - protein, 19 aa, Homo sapiens
CYFDDSSNVICKKYRGSGS

SEQ ID NO: 26 - protein, 26 aa, Homo sapiens
ECRDLGWQDWCCFDDSSNVICKKYRS

SEQ ID NO: 27 - protein, 28 aa, Homo sapiens
ECRDLGWQDWCAACFDDSSNVICKKYRS

SEQ ID NO: 28 - protein, 19 aa, Homo sapiens
EAACYFDDSSNVICKKYRS

SEQ ID NO: 29 - protein, 454 aa, Homo sapiens
MHLTVFLLKGIVGFLWSCWVLVGYAKGGLGDNHVHSSFIYRRLRNHERREIQREILSILGLPHRPRPFSPGKQASSAPLFMLDLYNAMTNEENPEESEYSVRASLAEETRGARKGYPASPNGYPRRIQLSRTTPLTTQSPPLASLHDTNFLNDADMVMSFVNLVERDKDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVRAANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 30 - protein, 34 aa, Homo sapiens
CKKHELYVSFRDLGWQDWIIAPEGYAAFYCDGEC

SEQ ID NO: 31 - protein, 33 aa, Homom sapiens
SFPLNAHMNATNHAIVQTLVHLMFPDHVPKPCC

SEQ ID NO: 32 - protein, 34 aa, Homo sapiens
APTKLNAISVLYFDDSSNVILKKYRNMVVRSCGC

SEQ ID NO: 33 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 34 - protein, 34 aa, Homo sapiens
CRKHELYVSFQDLGWQDWIIAPKGYAANYCDGEC

SEQ ID NO: 35 - protein, 33 aa, Homo sapiens
SFPLNAHMNATNHAIVQTLVHLMNPEYVPKPCC

SEQ ID NO: 36 - protein, 34 aa, Homo sapiens
APTKLNAISVLYFDDNSNVILKKYRNMVVRACGC

SEQ ID NO: 37 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 38 - protein, 34 aa, Homo sapiens
CKRHPLYVDFSDVGWNDWIVAPPGYHAFYCHGEC

SEQ ID NO: 39 - protein, 32 aa, Homo sapiens
PFPLADHLNSTNHAIVQTLVNSVNSKIPKACC

SEQ ID NO: 40 - protein, 34 aa, Homo sapiens
VPTELSAISMLYLDENEKVVLKNYQDMVVEGCGC

SEQ ID NO: 41 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 42 - protein, 34 aa, Homo sapiens
CRRHSLYVDFSDVGWNDWIVAPPGYQAFYCHGDC

SEQ ID NO: 43 - protein, 32 aa, Homo sapiens
PFPLADHLNSTNHAIVQTLVNSVNSSIPKACC

SEQ ID NO: 44 - protein, 34 aa, Homo sapiens
VPTELSAISMLYLDEYDKVVLKNYQEMVVEGCGC

SEQ ID NO: 45 - protein, 402 aa, Homo sapiens [sequence omitted]

SEQ ID NO: 46 - protein, 34 aa, Homo sapiens
CRRHELYVSFQDLGWLDWVIAPQGYSAYYCEGEC

SEQ ID NO: 47 - protein, 33 aa, Homo sapiens
SFPLDSCMNATNHAILQSLVHLMKPNAVPKACC

SEQ ID NO: 48 - protein, 34 aa, Homo sapiens
APTKLSATSVLYYDSSNNVILRKHRNMVVKACGC

SEQ ID NO: 49 - protein, 372 aa, Homo sapiens
MPPPQQGPCGHHLLLLLALLLPSLPLTRAPVPPGPAAALLQALGLRDEPQGAPRLRPVPPVMWRLFRRRDPQETRSGSRRTSPGVTLQPCHVEELGVAGNIVRHIPDRGAPTRASEPVSAAGHCPEWTVVFDLSAVEPAERPSRARLELRFAAAAAAAPEGGWELSVAQAGQGAGADPGPVLLRQLVPALGPPVRAELLGAAWARNASWPRSLRLALALRPRAPAACARLAEASLLLVTLDPRLCHPLARPRRDAEPVLGGGPGGACRARRLYVSFREVGWHRWVIAPRGFLANYCQGQCALPVALSGSGGPPALNHAVLRALMHAAAPGAADLPCCVPARLSPISVLFFDNSDNVVLRQYEDMVVDECGCR

SEQ ID NO: 50 - protein, 34 aa, Homo sapiens
CRARRLYVSFREVGWHRWVIAPRGFLANYCQGQC

SEQ ID NO: 51 - protein, 37 aa, Homo sapiens
ALPVALSGSGGPPALNHAVLRALMHAAAPGAADLPCC

SEQ ID NO: 52 - protein, 34 aa, Homo sapiens
VPARLSPISVLFFDNSDNVVLRQYEDMVVDECGC

SEQ ID NO: 53 - protein, 501 aa, Homo sapiens
MRLPKLLTFLLWYLAWLDLEFICTVLGAPDLGQRPQGTRPGLAKAEAKERPPLARNVFRPGGHSYGGGATNANARAKGGTGQTGGLTQPKKDEPKKLPPRPGGPEPKPGHPPQTRQATARTVTPKGQLPGGKAPPKAGSVPSSFLLKKAREPGPPREPKEPFRPPPITPHEYMLSLYRTLSDADRKGGNSSVKLEAGLANTITSFIDKGQDDRGPVVRKQRYVFDISALEKDGLLGAELRILRKKPSDTAKPAAPGGGRAAQLKLSSCPSGRQPASLLDVRSVPGLDGSGWEVFDIWKLFRNFKNSAQLCLELEAWERGRAVDLRGLGFDRAARQVHEKALFLVFGRTKKRDLFFNEIKARSGQDDKTVYEYLFSQRRKRRAPLATRQGKRPSKNLKARCSRKALHVNFKDMGWDDWIIAPLEYEAFHCEGLCEFPLRSHLEPTNHAVIQTLMNSMDPESTPPTCCVPTRLSPISILFIDSANNVVYKQYEDMVVESCGCR

SEQ ID NO: 54 - protein, 34 aa, Homo sapiens
CSRKALHVNFKDMGWDDWIIAPLEYEAFHCEGLC

SEQ ID NO: 55 - protein, 33 aa, Homo sapiens
EFPLRSHLEPTNHAVIQTLMNSMDPESTPPTCC

SEQ ID NO: 56 - protein, 34 aa, Homo sapiens
VPTRLSPISILFIDSANNVVYKQYEDMVVESCGC

SEQ ID NO: 57 - protein, 436 aa, Homo sapiens
RASAELGSAKGMRTRKEGRMPRAPRENATAREPLDRQEPPPRPQEEPQRRPPQQPEAREPPGRGPRLVPHEYMLSIYRTYSIAEKLGINASFFQSSKSANTITSFVDRGLDDLSHTPLRRQKYLFDVSTLSDKEELVGADVRLFRQAPAALAPPAAAPLAALRLPVAPAAGSAEPGPAGAPRPGWEVFDVWRGLRPQPWKQLCLELRAAWGGEPGAAEDEARTPGPQQPPPPDLRSLGFGRRVRTPQERALLVVFSRSQRKTLFAEMREQLGSATEVVGPGGGAEGSGPPPPPPPPPPSGTPDAGLWSPSPGRRRRTAFASRHGKRHGKKSRLRCSKKPLHVNFKELGWDDWIIAPLEYEAYHCEGVCDFPLRSHLEPTNHAIIQTLMNSMDPGSTPPSCCVPTKLTPISILYIDAGNNVVYNEYEEMVVESCGCR

SEQ ID NO: 58 - protein, 34 aa, Homo sapiens
CSKKPLHVNFKELGWDDWIIAPLEYEAYHCEGVC

SEQ ID NO: 59 - protein, 33 aa, Homo sapiens
DFPLRSHLEPTNHAIIQTLMNSMDPGSTPPSCC

SEQ ID NO: 60 - protein, 34 aa, Homo sapiens
VPTKLTPISILYIDAGNNVVYNEYEEMVVESCGC

SEQ ID NO: 61 - protein, 151 aa, Mus musculus
RRRRRTALAGTRGAQGSGGGGGGGGGGGGGGGGGGGGAGRGHGRRGRSRCSRKSLHVDFKELGWDDWIIAPLDYEAYHCEGVCDFPLRSHLEPTNHAIIQTLLNSMAPDAAPASCCVPARLSPISILYIDAANNVVYKQYEDMVVEACGCR

SEQ ID NO: 62 - protein, 34 aa, Mus musculus
CSRKSLHVDFKELGWDDWIIAPLDYEAYHCEGVC

SEQ ID NO: 63 - protein, 33 aa, Mus musculus
DFPLRSHLEPTNHAIIQTLLNSMAPDAAPASCC

SEQ ID NO: 64 - protein, 34 aa, Mus musculus
VPARLSPISILYIDAANNVVYKQYEDMVVEACGC

(19) United States
(12) Patent Application Publication
(10) Pub. No.: US 2003/0185792 A1
(43) Pub. Date: Oct. 2, 2003

(54) Morphogen analogs of bone morphogenic proteins

(75) Inventors: Peter C. Keck, Millbury, MA (US); Dattatreyamurty Bosukonda, Shrewsbury, MA (US)

Correspondence Address: ROPES & GRAY, ONE INTERNATIONAL PLACE, BOSTON, MA, 02110-2624, US

(73) Assignee: Curis, Inc., Cambridge, MA

(21) Appl. No.: 10/164,279

(22) Filed: Jun. 6, 2002

Related U.S. Application Data
(63) Continuation-in-part of application No. 09/791,946, filed on Feb. 22, 2001.
Application No. 09/791,946 is a continuation of application No. 08/786,284, filed on Jan. 22, 1997, now Pat. No. 6,273,598.
Application No. 08/786,284 is a continuation-in-part of application No. 08/589,552, filed on Jan. 22, 1996, now abandoned.
(60) Provisional application No. 60/354,820, filed on Feb. 5, 2002.
(60) Provisional application No. 60/371,298, filed on Apr. 10, 2002.
(60) Provisional application No. 60/296,291, filed on Jun. 6, 2001.

(51) Int. Cl. A61K 38/19; A61K 38/18; A61K 38/12; A61K 48/00; C12N 9/99

(52) U.S. Cl. 424/85.1; 514/9; 514/44; 514/12; 435/184

(57) ABSTRACT

The present invention relates to morphogen analogs, particularly analogs of a BMP, such as OP-1, that are agonists or antagonists of a BMP, such as OP-1, biological activity.

### RELATED APPLICATION

This application is a continuation-in-part of U.S. patent application Ser. No. 09/791,946, filed Feb. 22, 2001, which is a continuation of U.S. patent application Ser. No. 08/786,284, filed Jan. 22, 1997, now U.S. Pat. No. 6,273,598, which is a continuation-in-part of U.S. patent application Ser. No. 08/589,552, filed Jan. 22, 1996, now abandoned, which claims the benefit of U.S. Provisional Application No. 60/371,298, filed Apr. 10, 2002, U.S. Provisional Application No. 60/354,820, filed Feb. 5, 2002, and U.S. Provisional Application No. 60/296,291, filed Jun. 6, 2001, the specifications of all of which are hereby incorporated by reference in their entirety.

### FIELD OF THE INVENTION

The present invention relates generally to methods and compositions for designing, identifying, and producing compounds useful as tissue morphogenic protein analogs. More specifically, the invention relates to structure-based methods and compositions useful in designing, identifying, and producing molecules which act as functional mimetics of bone morphogenic proteins, such as osteogenic protein-1 (OP-1).

### BACKGROUND OF THE INVENTION

Cell differentiation is the central characteristic of tissue morphogenesis, which initiates during embryogenesis and continues to various degrees throughout the life of an organism in adult tissue repair and regeneration mechanisms. The degree of morphogenesis in adult tissue varies among different tissues and is related, among other things, to the degree of cell turnover in a given tissue.

The cellular and molecular events which govern the stimulus for differentiation of cells is an area of intensive research. In the medical and veterinary fields, it is anticipated that discovery of the factor or factors which control cell differentiation and tissue morphogenesis will advance significantly the ability to repair and regenerate diseased or damaged mammalian tissues and organs.

Particularly useful areas for human and veterinary therapeutics include reconstructive surgery, the treatment of tissue degenerative diseases including, for example, arthritis, emphysema, osteoporosis, cardiomyopathy, cirrhosis, degenerative nerve diseases, inflammatory diseases, and cancer, and in the regeneration of tissues, organs and limbs. In this and related applications, the terms “morphogenetic” and “morphogenic” are used interchangeably.

A number of different factors have been isolated in recent years that appear to play a role in cell differentiation. Recently, a distinct subfamily of the “superfamily” of structurally related proteins referred to in the art as the “transforming growth factor-β (TGF-β) superfamily of proteins” have been identified as true tissue morphogens.

The members of this distinct “subfamily” of true tissue morphogenic proteins share substantial amino acid sequence homology within their morphogenetically active C-terminal domains (at least 50% identity in the C-terminal 102 amino acid sequence), including a conserved six or seven cysteine skeleton, and share the in vivo activity of inducing tissue-specific morphogenesis in a variety of organs and tissues. The proteins apparently contact and interact with progenitor cells e.g., by binding suitable cell surface molecules, predisposing or otherwise stimulating the cells to proliferate and differentiate in a morphogenetically permissive environment. These morphogenic proteins are capable of inducing the developmental cascade of cellular and molecular events that culminate in the formation of new organ-specific tissue, including any vascularization, connective tissue formation, and nerve innervation as required by the naturally occurring tissue. The proteins have been shown to induce morphogenesis of both cartilage and bone, as well as periodontal tissues, dentin, liver, and neural tissue, including retinal tissue.

True tissue morphogenic proteins identified to date include proteins originally identified as bone inductive proteins. These include OP-1, (osteogenic protein-1, also referred to in related applications as “OP1”), its Drosophila homolog, 60A, with which it shares 69% identity in the C-terminal “seven cysteine” domain, and the related proteins OP-2 (also referred to in related applications as “OP2”) and OP-3, both of which share approximately 65-75% identity with OP-1 in the C-terminal seven cysteine domain, as well as bone morphogenic protein 5 (BMP5), BMP6, and its murine homolog, Vgr-1, all of which share greater than 85% identity with OP-1 in the C-terminal seven cysteine domain, and the BMP6 Xenopus homolog, Vg1, which shares approximately 57% identity with OP-1 in the C-terminal seven cysteine domain. Other bone inductive proteins include the CBMP2 proteins (also referred to in the art as BMP2 and BMP4) and their Drosophila homolog, DPP. Another tissue morphogenic protein is GDF-1 (from mouse). See, for example, PCT documents US92/01968 and US92/07358, the disclosures of which are incorporated herein by reference. In addition to the well established role for members of the TGFβ superfamily in osteogenesis, members of this family also possess potent chondrogenic activity. Exemplary, nonlimiting members of the TGFβ superfamily involved in chondrogenesis are CDMP1, CDMP2, and CDMP3. Note that these proteins are also referred to in the art as GDF5, GDF6, and GDF7, respectively. Members of the BMP/OP subfamily and the amino acid sequence identities (expressed as percentages) between selected members of the TGF-β superfamily are shown in FIG. 6.

As stated above, these true tissue morphogenic proteins are recognized in the art as a distinct subfamily of proteins different from other members of the TGF-β superfamily in that they share a high degree of sequence identity in the C-terminal domain and in that the true tissue morphogenic proteins are able to induce, on their own, the full cascade of events that result in formation of functional tissue rather than merely inducing formation of fibrotic (scar) tissue. TGF-β family members are involved in the development of organs and tissues derived from all three germ layers. Specifically, members of the family of morphogenic proteins are capable of all of the following in a morphogenetically permissive environment: stimulating cell proliferation and cell differentiation, and supporting the growth and maintenance of differentiated cells. The morphogenic proteins apparently also may act as endocrine, paracrine or autocrine factors.

The morphogenic proteins are capable of significant species “crosstalk.” That is, xenogenic (foreign species) homologs of these proteins can substitute for one another in functional activity. For example, dpp and 60A, two Drosophila proteins, can substitute for their mammalian homologs, BMP2/4 and OP-1, respectively, and induce endochondral bone formation at a non-bony site in a standard rat bone formation assay. Similarly, BMP2 has been shown to rescue a dpp⁻ mutation in Drosophila. In their native form, however, the proteins appear to be tissue-specific, each protein typically being expressed in or provided to one or only a few tissues or, alternatively, expressed only at particular times during development. For example, GDF-1 appears to be expressed primarily in neural tissue, while OP-2 appears to be expressed at relatively high levels in early (e.g., 8-day) mouse embryos. The endogenous morphogens may be synthesized by the cells on which they act, by neighboring cells, or by cells of a distant tissue, the secreted protein being transported to the cells to be acted on.

A particularly potent tissue morphogenic protein is OP-1. This protein and its xenogenic homologs are expressed in a number of tissues, primarily in tissues of urogenital origin, as well as in bone, mammary and salivary gland tissue, reproductive tissues, and gastrointestinal tract tissue. It is expressed also in different tissues during embryogenesis, its presence coincident with the onset of morphogenesis of that tissue.

The morphogenic protein signal transduction across a cell membrane appears to occur as a result of specific binding interaction with one or more cell surface receptors. Recent studies on cell surface receptor-binding of various members of the TGF-β protein superfamily suggests that the ligands mediate their activity by interaction with two different receptors, referred to as Type I and Type II receptors to form a hetero-complex. A cell surface bound beta-glycan also may enhance the binding interaction. The Type I and Type II receptors are both serine/threonine kinases, and share similar structures: an intracellular domain that consists essentially of the kinase, a short, extended hydrophobic sequence sufficient to span the membrane one time, and an extracellular domain characterized by a high concentration of conserved cysteines.

Morphogenic proteins are disulfide-linked dimers which are expressed as large precursor polypeptide chains containing a hydrophobic signal sequence, a long and relatively poorly conserved N-terminal pro region of several hundred amino acids, a cleavage site and a mature domain comprising an N-terminal region which varies among the family members and a more highly conserved C-terminal region. The C-terminal region, which is present in the processed mature proteins of all known morphogen family members, contains approximately 100 amino acids with a characteristic motif having a conserved six or seven cysteine skeleton. Each of the morphogenic proteins isolated to date are dimeric structures wherein the monomer subunits are held together by non-covalent interactions or by one or more disulfide bonds. The morphogenic proteins are active as dimeric proteins but are inactive as individual monomer subunits.

As a result of their biological activities, significant effort has been directed toward the development of morphogen-based therapeutics for treating injured or diseased mammalian tissue, including, for example, therapeutic compositions for inducing regenerative healing of bone defects such as fractures, as well as therapeutic compositions for preserving or restoring healthy metabolic properties in diseased bone tissue, e.g., osteopenic bone tissue. Complete descriptions of efforts to develop and characterize morphogen-based therapeutics for non-chondrogenic tissue applications in mammals, particularly humans, are set forth, for example, in: EP 0575,555; WO93/04692; WO93/05751; WO94/06399; WO94/03200; WO94/06449; WO94/10203; and WO94/06420, the disclosures of each of which are incorporated herein by reference.

Certain difficulties may be experienced upon administration of naturally isolated or recombinantly produced morphogenic proteins to a mammal. These difficulties may include, for example, loss of morphogenic activity due to disassociation of the biologically active morphogen dimer into its inactive monomer subunits, and/or handling problems due to low solubility under physiological conditions.

Accordingly, a need remains for the identification of morphogen analogs, which mimic or enhance the physiological effects of a morphogenic protein, for example OP-1. Additionally, a need exists for the identification of morphogen analogs which antagonize the activity or physiological effects of a morphogenic protein.

### SUMMARY OF THE INVENTION

The present invention is based, in part, upon the X-ray crystallographic determination of the three-dimensional structure of mature, dimeric human osteogenic protein-1 (hOP-1). The three-dimensional structure of hOP-1 has been resolved to 2.3 Å. See, for example, U.S. Pat. No. 6,273,598, incorporated herein by reference. With this disclosure, the skilled artisan is provided with sets of atomic co-ordinates for use in conventional computer aided design (CAD) methodologies to identify or design protein or peptide analogs of OP-1, or alternatively, to identify or design small organic molecules that functionally mimic OP-1. Because several members of the BMP family of proteins share significant sequence similarity to OP-1, this structural data can also be used to design antagonistic and agonistic analogs of such BMP proteins.

In one embodiment, a processor can use these coordinates to design a morphogen analog, or a morphogen antagonist for example, a protein, peptide or small organic molecule, having a three-dimensional shape and preferably, in addition, a solvent accessible surface corresponding to at least a portion of a human BMP, such as OP-1 and competent to mimic a specific activity of a human BMP, such as OP-1. The analogs may be modified, morphogenically active hOP-1 protein dimers, or fragments or truncated analogs thereof, peptides or small organic molecules. Preferably the analogs have enhanced therapeutic value, for example, by being more stable and/or more soluble under physiological conditions than naturally occurring hOP-1, or, for example, by having enhanced tissue targeting specificity, enhanced biodistribution or a reduced clearance rate in the body.

As used herein, with respect to OP-1 (or related morphogens), or with respect to a region of OP-1, the phrase “at least a portion of the three-dimensional structure of” or “at least a portion of” is understood to mean a portion of the three-dimensional surface structure of the morphogen, or region of the morphogen, including charge distribution and hydrophilicity/hydrophobicity characteristics, formed by at least three, more preferably at least three to ten, and most preferably at least ten contiguous amino acid residues of the OP-1 monomer or dimer. The contiguous residues forming such a portion may be residues which form a contiguous portion of the primary structure of the OP-1 molecule, residues which form a contiguous portion of the three-dimensional surface of the OP-1 monomer, residues which form a contiguous portion of the three-dimensional surface of the OP-1 dimer, or a combination thereof. Thus, the residues forming a portion of the three-dimensional structure of OP-1 need not be contiguous in the primary sequence of the morphogen but, rather, must form a contiguous portion of the surface of the morphogen monomer or dimer. In particular, such residues may be non-contiguous in the primary structure of a single morphogen monomer or may comprise residues from different monomers in the dimeric form of the morphogen. As used herein, the residues forming “a portion of the three-dimensional structure of” a morphogen, or “a portion of” a morphogen, form a contiguous three-dimensional surface in which each atom or functional group forming the portion of the surface is separated from the nearest atom or functional group forming the portion of the surface by no more than 40 Å, preferably by no more than 20 Å, more preferably by no more than 5-10 Å, and most preferably by no more than 1-5 Å.

As used herein, the term “morphogen analog”, is understood to mean any molecule capable of mimicking the receptor-binding activity and/or inducing a receptor-mediated downstream biological effect characteristic of a morphogenic protein, such as a BMP, e.g., OP-1. Inducing alkaline phosphatase activity is a characteristic biological effect. The analog may be a protein, peptide, or non-peptidyl based organic molecule. Accordingly, the term morphogen analog embraces any substance having such BMP-like or OP-1-like activity, regardless of the chemical or biochemical nature thereof. The present morphogen analog can be a simple or complex substance produced by a living system or through chemical or biochemical synthetic techniques, or discovered by high-throughput screening. It can be a large molecule, e.g., a modified hOP-1 dimer produced by recombinant DNA methodologies, or a small molecule, e.g., an organic molecule prepared de novo according to the principles of rational drug design. It can be a substance which is a mutein (or mutant protein) of hOP-1, a substance that structurally resembles a solvent-exposed surface epitope of hOP-1 and binds an OP-1 specific receptor, or a substance that otherwise stimulates an OP-1 specific receptor displayed on the surface of an OP-1 responsive cell.

As used herein, the terms “BMP or BMP-like biological activity” are understood to mean any biological activities known to be induced or enhanced by a BMP. BMP-like biological activities include, but are not limited to, stimulating proliferation of progenitor cells; stimulating differentiation of progenitor cells; stimulating proliferation of differentiated cells; and supporting growth and maintenance of differentiated cells. The term “progenitor cells” includes uncommitted cells, preferably of mammalian origin that are competent to differentiate into one or more specific types of differentiated cells, depending on their genomic repertoire and the tissue specificity of the permissive environment where morphogenesis is induced. Specifically, with regard to bone, cartilage, nerve, and liver tissue, the BMP-stimulated morphogenic cascade culminates in the formation of new or regenerative differentiated tissue appropriate to the selected local environment. BMP-mediated morphogenesis, therefore, differs significantly from simple reparative healing processes in which scar tissue (e.g., fibrous connective tissue) is formed and fills a lesion or other defect in differentiated functional tissue.

As used herein, the term “BMP” is understood to refer to morphogenic proteins belonging to the TGF-β superfamily, and characterized by the presence of a six or seven cysteine skeleton within a C-terminal region which is approximately 50% identical among these related family members (see FIG. 6). This include proteins referred to in the art as a bone morphogenetic protein (bmp) such as BMP2, BMP4, BMP5, BMP6, BMP7/OP1, BMP8/OP2, BMP9, BMP10, etc, as well as related proteins generally referred to in the art by different names but understood to meet the characteristics outlined herein for a “BMP”. Non-limiting examples of such related proteins include GDF1, GDF2, GDF3, GDF5, GDF6, GDF7, as well as the proteins depicted in FIG. 6.

As used herein a “morphogen antagonist” includes both a molecule competent to mimic BMP receptor-binding activity but which cannot induce a receptor-mediated downstream effect, as well as a molecule which binds to a BMP morphogen and prevents said morphogen from binding to its receptor (i.e., the antagonist acts as a dominant negative).

In a related aspect, the invention provides a method of producing a morphogen analog that mimics or enhances a BMP or BMP-like biological activity. The method comprises the steps of: (a) providing a molecular model defining a three-dimensional shape representative of at least a portion of human OP-1, (b) identifying a compound having a three-dimensional shape corresponding to the three-dimensional shape representative of at least the portion of human OP-1; and (c) producing the compound identified in step (b). The method can comprise the additional step of testing the compound in a biological system to determine whether the resultant candidate compound mimics or agonizes the biological activity of a BMP, such as OP-1. It is contemplated that, in the aforementioned method, step (a) and/or (b) may be performed by means of an electronic processor using commercially available software packages.

It is contemplated that, upon determination of whether the candidate compound modulates a BMP, such as OP-1, activity, the candidate compound can be iteratively improved using conventional CAD and/or rational drug design methodologies, well known and thoroughly documented in the art. Furthermore, it is contemplated that the resultant compound identified thus far, may be produced in a commercially useful quantity for administration into a mammal.

In another embodiment, the invention provides means for creating an analog with altered receptor-binding characteristics. For example, provided with the structure, charge distribution, and solvent accessible surface information pertaining to the putative receptor-binding site, one can alter or modify receptor-binding specificity and avidity. In one embodiment, amino acid replacements in this region are made with reference to the corresponding amino acids of other known morphogens, disclosed for example, in WO94/06449 or WO93/05751.

In certain embodiments, the present invention relates to a peptide that antagonizes a biological activity of a BMP, such as OP-1, wherein the peptide includes a peptide sequence having from 6 to 50 amino acid residues, or from 8 to 40, or even from 8 to 30 amino acid residues, including at least three, four, five, six, seven, or eight contiguous amino acids of SEQ No. 1, 2, 30, 31, 34, 35, 38, 39, 42, 43, 46, 47, 50, 51, 54, 55, 58, 59, 62, or 63. In certain embodiments, at least 90% of the amino acid residues of the peptide sequence are contiguous amino acid residues of SEQ ID No. 1, 2, 30, 31, 34, 35, 38, 39, 42, 43, 46, 47, 50, 51, 54, 55, 58, 59, 62, or 63. In certain embodiments, the peptide sequence comprises a sequence at least 90% identical, or at least 90% identical to at least one of SEQ ID Nos. 5, 6, 7, 9, 11, 12, 13, and 14. The peptide may be a competitive, non-competitive, uncompetitive, reversible, or irreversible inhibitor of the biological activity.

In certain embodiments, the peptide sequence has at least two non-adjacent cysteine residues that are joined by a disulfide bond to form a ring, e.g., including from 8 to 30 amino acids, or from 8 to 20 amino acids. In certain such embodiments, the cysteine residues are located on either end of the peptide sequence, such that the ring comprises all of the amino acid residues of the peptide backbone.

In certain embodiments, the present invention provides a peptide having a peptide sequence comprising any one of SEQ ID Nos. 5, 6, 7, 9, 11, 12, 13, and 14, wherein at least two cysteine residues are joined by a disulfide bond to form a ring.

In certain embodiments, the invention provides a cyclic peptide that antagonizes a biological activity of a BMP, such as OP-1, wherein the peptide comprises a peptide sequence having a cysteine residue on each end and including at least three, or at least ten, contiguous amino acids of SEQ ID No. 1, 2, 30, 31, 34, 35, 38, 39, 42, 43, 46, 47, 50, 51, 54, 55, 58, 59, 62, or 63, and wherein the cysteine residues are linked together by a disulfide bond.

In another aspect, the invention provides a peptide that agonizes a biological activity of a BMP, such as OP-1, wherein the peptide includes a peptide sequence having between 6 and 50 amino acid residues, or from 8 to 40, or even from 8 to 30 amino acid residues, including at least five contiguous amino acids of SEQ No. 3, 32, 36, 40, 44, 48, 52, 56, 60, or 64. In certain embodiments, at least 90% of the amino acid residues of the peptide sequence are contiguous amino acid residues of SEQ ID No. 3, 32, 36, 40, 44, 48, 52, 56, 60, or 64. In certain embodiments, the peptide sequence comprises a sequence at least 90% identical or even 95% identical to at least one of SEQ ID Nos. 16, 17, 19, 21, 22, 23, 24, 25, 26, 27, and 28.

In certain embodiments, the peptide sequence has at least two non-adjacent cysteine residues that are joined by a disulfide bond to form a ring, e.g., including from 8 to 30 amino acids, or from 8 to 20 amino acids. In certain such embodiments, the cysteine residues are located on either end of the peptide sequence, such that the ring comprises all of the amino acid residues of the peptide backbone.

In certain embodiments, the invention provides a peptide having a peptide sequence comprising any one of SEQ ID Nos. 16, 17, 19, 21, 22, 23, 24, 25, 26, 27, and 28, wherein at least two cysteine residues are joined by a disulfide bond to form a ring.

In certain other embodiments, the invention provides a cyclic peptide that agonizes a biological activity of a BMP, such as OP-1, wherein the peptide comprises a BMP peptide sequence bounded by a cysteine residue on each end and consisting essentially of at least three, or at least ten, contiguous amino acids of SEQ ID No. 3, 32, 36, 40, 44, 48, 52, 56, 60, or 64, and wherein the cysteine residues are linked together by a disulfide bond.

In yet another aspect, the invention provides a peptide that brings together a Type I and a Type II receptor, comprising a peptide backbone having a first peptide sequence having a cysteine residue on each end and including at least three contiguous amino acids of SEQ ID No. 1, 2, 30, 31, 34, 35, 38, 39, 42, 43, 46, 47, 50, 51, 54, 55, 58, 59, 62, or 63, and wherein the cysteine residues are linked together by a disulfide bond; and a second peptide sequence having a cysteine residue on each end and including at least three contiguous amino acids of SEQ ID No. 3, 32, 36, 40, 44, 48, 52, 56, 60, or 64, and wherein the cysteine residues are linked together by a disulfide bond. Preferably, the first and second peptide sequences are linked by an amino acid chain of from 0 to 20 residues, preferably from 0 to 5 residues.

In certain embodiments, the cyclic peptides described above consist exclusively of a peptide backbone terminating in cysteine residues at either end, the cysteine residues being linked by disulfide bonds. In certain other embodiments, such peptides further include other linear or cyclic fragments of a BMP, such as OP-1, (e.g., from 1 to 20 residues, from 1 to 10 residues, or even from 1 to 5 residues), and/or other heterologous peptide sequences (e.g., from 1 to 20 residues, from 1 to 10 residues, or even from 1 to 5 residues).

In certain embodiments, the linear peptides described above may include additional fragments of a BMP, such as OP-1, and/or heterologous peptide sequences.

In another aspect, the invention provides peptidomimetics of any of the above peptides, e.g., wherein one or more amide bonds is replaced with a moiety that improves the peptide's resistance to hydrolysis.

In yet another aspect, the invention provides a nucleic acid sequence encoding any one of the peptides described above. Such nucleic acid sequences may be employed in vectors for transfecting cells and producing recombinant peptides, e.g., in vivo or in vitro.

In still another aspect, the invention provides pharmaceutical preparations of any of the above peptides, nucleic acids, or peptidomimetics. Preparations comprising BMP agonists may further include a BMP protein, e.g., preparations comprising OP-1 agonists may further include OP-1 protein. Such compositions may comprise a subtherapeutic dose of the BMP protein, e.g., less than half, less than a quarter, or even less than a tenth of the ED₅₀ for the protein itself, because the agonist increases the efficacy of the subtherapeutic dose to a therapeutically useful level.

In yet another aspect, the present invention provides a method for inhibiting growth, differentiation, or proliferation of a cell, such as a bone, cartilage, nerve, or liver cell, comprising contacting the cell with a BMP antagonizing peptide as described above.

Similarly, the present invention also provides a method for promoting growth, differentiation, or proliferation of a cell, such as a bone, cartilage, nerve, or liver cell, comprising contacting the cell with a BMP-agonizing peptide as described above. In certain such embodiments, the method includes contacting the cell with a BMP, such as OP-1. The cell may be contacted with both agents simultaneously (e.g., in a single pharmaceutical preparation), substantially simultaneously (e.g., in two or more preparations administered within a short period of time, e.g., 2 hours), or otherwise as part of a single therapeutic regimen such that the agonizing peptide increases the response of the cell to the BMP/OP-1 protein. In certain other embodiments, the method includes contacting the cell with a BMP-agonizing peptide, in combination with another agent which promotes the growth, differentiation, or proliferation of a cell, wherein that agent is not a member of the TGF-β superfamily. Exemplary non-TGF-β agents which promote growth, differentiation, or proliferation include growth factors (such as FGF, PDGF, EGF, and NGF family members), morphogens (such as hedgehog, Wnt, and Notch family members), and cytokines. Additionally, said non-TGF-β agent may be a peptide or small molecule which agonizes the activity of the growth factor, morphogen, or cytokine. The cell may be contacted with both agents simultaneously (e.g., in a single pharmaceutical preparation), substantially simultaneously (e.g., in two or more preparations administered within a short period of time, e.g., 2 hours), or otherwise as part of a single therapeutic regimen such that the agonizing peptide increases the response of the cell to the non-TGF-β superfamily agent.

Similarly, the peptides, peptidomimetics, nucleic acids, and formulations may be used to treat diseases or conditions in a patient, such as a mammal, e.g., a human. Suitable diseases, conditions, and indications are described in PCT Application WO 92/15323 on pages 6-10 and 61-66. WO 92/15323 is incorporated by reference in its entirety, and specifically the recited sections relating to therapeutic indications.

The present invention also contemplates gene therapy protocols related to the above methods, in which the cell is transfected with a nucleic acid encoding one of the above-described peptides.

In yet another aspect, the present invention contemplates the preparation, testing, and use of analogs of OP-1-like proteins, such as BMPs, e.g., peptides which incorporate portions of BMP proteins analogous to the portions of OP-1 set forth above as described in detail herein.

The foregoing and other objects, features and advantages of the present invention will be made more apparent from the following detailed description of preferred embodiments of the invention.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

The objects and features of the invention may be better understood by reference to the drawings described below, wherein like-referenced features identify common features in corresponding figures.

FIG. 1A is a simplified line drawing useful in describing the structure of a monomeric subunit of hOP-1. See the Summary of the Invention, infra, for explanation.

FIGS. 1B, 1C, and 1D are monovision ribbon tracings of the respective peptide backbones of hOP-1 finger-1, heel, and finger-2 regions.

FIGS. 1E and 1F are schematic representations of monomeric and dimeric forms of hOP-1, respectively, as represented by a left hand motif.

FIG. 2 is a schematic drawing of a monomeric subunit of hOP-1. The hOP-1 cysteine knot comprising three disulfide bonds constitutes the core of the hOP-1 monomer subunit. Two disulfide bonds which connect residues Cys 67-Cys 136 and Cys 71-Cys 138 produce an eight residue ring through which the third disulfide bond which connects residues Cys 38-Cys 104 passes. Four strands of antiparallel β-sheet, which emanate from the knot, form the two finger-like projections. An α-helix located on the opposite end of the knot, lies perpendicular to the axis of the two fingers thereby forming the heel. The N-terminus of the monomer subunit remains unresolved. The β-sheets are displayed as arrows and labeled from β1 through β8. The α-helix is displayed as a tube and labeled 1. The intra-subunit disulfide bonds that constitute the cysteine knot are shown in solid lines. Starting from Gln36 (“N₃₆”), the first residue shown in this figure, the amino acid residues that produce secondary structure in the Finger 1 region include: Lys 39-His 41 (β1), Tyr 44-Ser 46 (β2), Glu 60-Ala 63 (β3), Tyr 65-Glu 70 (β4); the amino acid residues that produce secondary structure in the Finger 2 region include: Cys 103-Asn 110 (β5); Ile 112-Asp 118 (β6); Asn 122-Tyr 128 (β7); Val 132-His 139 (β8); and the amino acid residues that produce secondary structure in the heel region include: Thr 82-Ile 94(α1).

FIG. 3 is a structure-based sequence alignment of the hOP-1 and TGF-β2 finger-1, heel, and finger-2 regions. Amino acid residues in the heel regions that constitute inter-chain contacts in the dimers of hOP-1 and TGF-β2 are highlighted as white on black. Amino acid residues in the finger-1 and finger-2 regions that contact the other chain are highlighted as black on gray. In hOP-1 and TGF-β2, the amino acids located at the same residue positions constitute the inter-chain contacts.

FIGS. 4A and 4B are stereo peptide backbone ribbon trace drawings illustrating the three-dimensional shape of hOP-1: A) from the “top” (down the two-fold axis of symmetry between the subunits) with the axes of the helical heel regions generally normal to the paper and the axes of each of the finger 1 and finger 2 regions generally vertical, and B) from the “side” with the two-fold axis between the subunits in the plane of the paper, with the axes of the heels generally horizontal, and the axes of the fingers generally vertical. The hOP-1 monomer has an accessible non-polar surface area of approximately 4394 Å², while that for the dimer is approximately 6831 Å² resulting in a hidden area upon dimerization of approximately 979 Å² per monomer. The reader is encouraged to view the stereo alpha carbon trace drawings in wall-eyed stereo, for example, using a standard stereo viewer device, to more readily visualize the spatial relationships of amino acids sequences in the morphogen analog design.

FIG. 5A is a backbone ribbon trace drawing illustrating the hOP-1 dimer comprising the two hOP-1 monomer subunits resolved to 2.8 Å. One monomer subunit is shown in green and the other monomer subunit is shown in gold. Amino acid residues disposed within the purported receptor-binding domain having solvent accessible side chains are shown as atomic spheres. The tips of the finger 1 and finger 2 regions of one OP-1 monomeric subunit and a loop at the C-terminal end of the heel of the other OP-1 monomeric subunit are believed to constitute the receptor-binding domain. Amino acids located at positions of variable amino acid sequence shown in white while amino acids located at more conserved positions are shown in red.

FIGS. 5B and 5C are pictures showing the respective solvent accessible surfaces of OP-1 and TGF-β2 dimers colored based on their electrostatic potential. Surface regions having an electrostatic potential of −3 kT or less are shown in red while surface regions of +3 kT or greater are shown in blue. Neutral regions are shown in green or gold to correspond to the backbone ribbons shown in 5A.

FIG. 6 is a table showing an identity matrix for the TGF-β superfamily. The matrix comprises members of the TGF-β superfamily having an amino acid sequence identity relative to OP-1 of greater than 36%. In the matrix, the TGF-β superfamily members are placed in order of decreasing amino acid identity relative to OP-1. TGF-β2 has an amino acid sequence of identity of 36% relative to OP-1 and is positioned the bottom of the matrix. Boxes enclose families of sequences having 50% or higher identity with a majority of the other members of the family; with sequences having identities of 75% or higher are shown in gray. Recombinantly expressed OP/BMP family members which have been shown to make bone are denoted by a “+” in the left margin. In the left margin, TGF-β superfamily members with three-dimensional structures determined are highlighted white on black. The sequences are referenced in Kingsley (Kingsley. (1994) Genes and Development 8:133-146), except for the following: (UNIVIN (Stenzel el al. (1994) Develop. BioL 166:149-158), SCREW (Arora, et al. (1994) Genes and Dev. 8:2588-2601), BMP-9 (Wozney, et al. (1993) PCT/WO 93/00432, SEQ ID NO. 9), BMP-10 (Celeste et al (1994) PCT/WO 94/26893, SEQ ID NO. 1), GDF-5 (Storm et al. (1994) Nature 368:639-643) (also called CDMP-1 (Chang et al. (1994) J Biol. Chem. 269: 28227-28234), GDF-6 (Storm, et al. (1994) Nature 368:639-643), GDF-7 (Storm et al. (1994) Nature 368:639-643), CDMP-2 (Chang et al. (1994) J Biol. Chem. 269: 28227-28234), OP-3 (Ozkaynak et al. (1994) PCT/WO 94/10203, SEQ ID NO. 1), Inhibin Be (Hotten, et al. (1995) Bioch. Biophys. Res. Comm. 206:608-613), and GDF-10 (Cumningham, et al. (1995) Growth Factors 12:99-109). The disclosures of the aforementioned citations are incorporated herein by reference. Several sequences in the matrix have alternate names: OP-1 (BMP-7), BMP-2 (BMP-2a), BMP-4 (BMP-2b), BMP-6 (Vgr1), OP-2 (BMP-8), 60A (Vgr-D), BMP-3 (osteogenin), GDF-5 (CDMP-1, MP-52), GDF-6 (CDMP-2, BMP-13) and GDF-7 (CDMP-3, BMP-12).

FIGS. 7A, 7B, and 7C show the amino acid sequences defining the human OP-1 finger 1 (SEQ ID No. 1), heel (SEQ ID No. 2), and finger 2 (SEQ ID No. 3) regions, respectively. The amino acid residues having 40% or greater of their sidechain exposed to solvent are boxed, wherein the solvent accessible amino acid residues that are highly variable among the BMP/OP family of the TGF-β superfamily are identified by shaded boxes. The amino acid sequences shown in FIGS. 7A, 7B, and 7C together define the solvent accessible surfaces of dimeric hOP-1, according to the 2.8 Å resolution structure.

FIG. 8 is an amino acid sequence alignment showing the amino acid sequence of mature human OP-1 (SEQ ID No. 4), and peptides defining the finger-1, finger-2 and heel regions of human OP-1 (SEQ ID Nos. 5-28).

FIGS. 9A-9D are bar graphs illustrating the effect of finger-2 and heel peptides on the alkaline phosphatase activity of ROS cells incubated in either the presence or absence of soluble OP-1. FIGS. 9A, 9B, 9C, and 9D show the effect of peptides F2-2, F2-3, Hn-2 and Hn-3, respectively, on the alkaline phosphatase activity of ROS cells incubated in the presence (shaded bars) or absence of soluble OP-1 (unshaded bars).

FIGS. 10A and 10B are graphs showing the displacement of radiolabelled soluble OP-1 from ROS cell membranes by finger 1, finger 2, and heel peptides. FIG. 10A shows the displacement of radiolabelled OP-1 from ROS cell membranes by unlabeled soluble OP-1 (open circles and triangles), finger 2 peptide F2-2 (closed circles) and finger 2 peptide F2-3 (closed triangles). FIG. 10B shows the displacement of radiolabelled OP-1 from ROS cell membranes by unlabeled soluble OP-1 (open triangles), finger 1 peptide F1-2 (closed boxes), heel peptide H-n2 (open diamonds) and heel peptide H-c2 (open circles).

FIG. 11 is a graph showing the effect of intact OP-1 and truncated OP-1 preparations on stimulation of alkaline phosphatase activity in the ROS cell-based bio-assay.

FIG. 12 depicts various peptide analogs and shows their relationship to the sequence of OP-1.

FIG. 13 is a bar graph showing the effect of the OP-1 finger 1 loop region peptide on the basal (no OP-1) and OP-1 induced alkaline phosphatase activity in the ROS cell-based assay.

FIG. 14 is a bar graph showing the effect of OP-1 peptides and unlabeled OP-1 on the binding of a radio-iodinated OP-1 to ROS cell plasma membranes.

FIG. 15 is a bar graph showing the effect of OP-1 heel region C-terminal loop peptide on the basal (no OP-1) and OP-1 induced alkaline phosphatase activity in the ROS cell-based assay.

FIG. 16 is a bar graph showing the effect of OP-1 finger 2 loop peptide on the basal (no OP-1) and OP-1 induced alkaline phosphatase activity in the ROS cell-based assay.

FIG. 17 is a bar graph showing the effect of OP-1 finger 2 loop peptide and unlabeled OP-1 on the binding of radio-iodinated OP-1 to ROS cell plasma membranes.

FIG. 18 is a graph showing the effect of the OP-1 finger 2 peptide F2-3 and of unlabeled OP-1 on the binding of radio-iodinated OP-1 to ROS cell plasma membrane bound receptors.

FIG. 19 is a bar graph showing the effect of the OP-1 finger 2 peptide F2-3 on the basal (No OP-1) and the OP-1 induced alkaline phosphatase activity in a ROS cell bioassay. Significant differences in the level of response to OP-1 in the presence of different concentrations of peptide were statistically analyzed and the corresponding p values are indicated. The level of OP-1 response in the absence of peptide is approximately 60% of maximum.

FIG. 20 is a bar graph demonstrating that the Finger 2 loop region of OP-1 with extension (F2-3) binds to ECD of DAF-4 by a rapid solid phase assay.

FIG. 21 is a ligand blot demonstrating OP-1 binding and size (Mᵣ) of ROS cell receptor (type II), where A is the binding of radio-labeled OP-1 to the receptor in the absence of excess unlabeled OP-1 and B is the binding of radio-labeled OP-1 to the receptor in the presence of excess unlabeled OP-1.

FIG. 22 is a ligand blot demonstrating OP-1 binding and size (Mᵣ) of ECD of DAF-4 receptor, where A is the binding of radio-labeled OP-1 to the receptor in the absence of excess unlabeled OP-1 and B is the binding of radio-labeled OP-1 to the receptor in the presence of excess unlabeled OP-1.

FIG. 23 is a graph showing the effect of OP-1 on stimulation of alkaline phosphatase activity in ROS cell assay.

FIGS. 24A and 24B are graphs showing the effect of OP-1 on stimulation of alkaline phosphatase activity and osteocalcin synthesis respectively in osteoblast-enriched cultures.

FIG. 25 is a graph showing the effect of OP-1 and other BMP preparations on dendritic growth in rat sympathetic neurons.

FIG. 26 is a graph showing the effect of OP-1 on bone-forming activity in rats, as determined by the calcium content of the implant.

FIGS. 27A and 27B are photomicrographs of implants in rats showing negative control (bone matrix alone) and OP-1 induced bone formation by day 14.

### DETAILED DESCRIPTION OF PREFERRED EMBODIMENTS

I. Introduction

Provided below is a detailed description of how to use coordinates in a database or biological techniques such as combinatorial mutagenesis to design a morphogen analog or structural variant of interest. Amino acid sequences as exemplary templates are provided as examples for designing, identifying, and producing a BMP analog, such as an OP-1 analog, using one of the OP-1 atomic coordinate databases. Specifically contemplated herein as useful analogs include: small amino molecules which mimic the receptor-binding region of the protein; analogs having enhanced stability or solubility; analogs having reduced clearance rates from the body; or enhanced target tissue specificity. The reader will appreciate that these examples are merely exemplary. Given the disclosure of the coordinates, the three-dimensional structure, the use of the coordinates in a database, and the level of skill in the art today, still other analogs, not specifically recited herein, are contemplated and enabled by this disclosure. In particular, it will be appreciated that, given the disclosure herein, and the known amino acid sequences for other, closely related morphogens, the methods can be used to create other morphogen analogs of, for example, BMP2, BMP4, OP2, BMP5, BMP6, GDF5, GDF6, and GDF7.

II. Definitions

For convenience, certain terms employed in the specification, examples, and appended claims are collected here.

As used herein, the term “gene” or “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides.

The terms “protein”, “polypeptide” and “peptide” are used interchangeably herein when referring to a gene product, e.g., as may be encoded by a coding sequence.

As used herein, the term “transfection” means the introduction of a nucleic acid, e.g., an expression vector, into a recipient cell by nucleic acid-mediated gene transfer.

“Transcriptional regulatory sequence” is a generic term used throughout the specification to refer to DNA sequences, such as initiation signals, enhancers, and promoters, which induce or control transcription of protein coding sequences with which they are operably linked.

Operably linked is intended to mean that the nucleotide sequence is linked to a regulatory sequence in a manner which allows expression of the nucleotide sequence. Regulatory sequences are art-recognized and are selected to direct expression of the subject peptide. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990).

The term “gene construct” refers to a vector, plasmid, viral genome or the like which includes a coding sequence, can transfect cells, preferably mammalian cells, and can cause expression of the peptide (or polypeptide including such moieties) or peptidomimetic of the cells transfected with the construct.

The term “interact” as used herein is meant to include detectable interactions between molecules, such as can be detected using, for example, a yeast two hybrid assay or by immunoprecipitation. The term interact is also meant to include “binding” interactions between molecules. Interactions may be, for example, protein-protein, protein-nucleic acid, protein-small molecule or small molecule-nucleic acid in nature. Preferred binding affinities have a Kd of 10⁻⁶ M or less, preferably 10⁻⁸ or less, 10⁻⁹ or less, 10⁻¹⁰ or less, 10⁻¹¹ or less, or most preferably 10⁻¹² or less.

As used herein, the term “transfection” means the introduction of a nucleic acid, e.g., an expression vector, into a recipient cell by nucleic acid-mediated gene transfer. The term “transduction” is generally used herein when the transfection with a nucleic acid is by viral delivery of the nucleic acid. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally occurring form of the recombinant protein is disrupted.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The terms “chimeric”, “fusion” and “composite” are used to denote a protein, peptide domain or nucleotide sequence or molecule containing at least two component portions which are mutually heterologous in the sense that they are not, otherwise, found directly (covalently) linked in nature. More specifically, the component portions are not found in the same continuous polypeptide or gene in nature, at least not in the same order or orientation or with the same spacing present in the chimeric protein or composite domain. Such materials contain components derived from at least two different proteins or genes or from at least two non-adjacent portions of the same protein or gene. Composite proteins, and DNA sequences which encode them, are recombinant in the sense that they contain at least two constituent portions which are not otherwise found directly linked (covalently) together in nature.

A “patient” or “subject” to be treated by the subject method can mean either a human or non-human animal.

The term “amino acid residue” is known in the art. In general the abbreviations used herein for designating the amino acids and the protective groups are based on recommendations of the IUPAC-IUB Commission on Biochemical Nomenclature (see Biochemistry (1972) 11:1726-1732). In certain embodiments, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan.

The term “amino acid residue” further includes analogs, derivatives and congeners of any specific amino acid referred to herein, as well as C-terminal or N-terminal protected amino acid derivatives (e.g., modified with an N-terminal or C-terminal protecting group). For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups). For instance, the subject compound can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminopimelic acid, ornithine, or diaminobutyric acid. Other naturally occurring amino acid metabolites or precursors having side chains which are suitable herein will be recognized by those skilled in the art and are included in the scope of the present invention.

Also included are the (D) and (L) stereoisomers of such amino acids when the structure of the amino acid admits of stereoisomeric forms. The configuration of the amino acids and amino acid residues herein are designated by the appropriate symbols (D), (L) or (DL), furthermore when the configuration is not designated the amino acid or residue can have the configuration (D), (L) or (DL). It will be noted that the structure of some of the compounds of this invention includes asymmetric carbon atoms. It is to be understood accordingly that the isomers arising from such asymmetry are included within the scope of this invention. Such isomers can be obtained in substantially pure form by classical separation techniques and by sterically controlled synthesis. For the purposes of this application, unless expressly noted to the contrary, a named amino acid shall be construed to include both the (D) or (L) stereoisomers. D- and L-α-Amino acids are represented by the following Fischer projections and wedge-and-dash drawings. In the majority of cases, D- and L-amino acids have R- and S-absolute configurations, respectively.

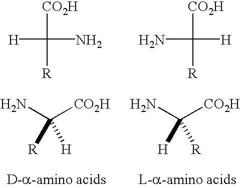

A “reversed” or “retro” peptide sequence as disclosed herein refers to that part of an overall sequence of covalently bonded amino acid residues (or analogs or mimetics thereof) wherein the normal carboxyl-to amino direction of peptide bond formation in the amino acid backbone has been reversed such that, reading in the conventional left-to-right direction, the amino portion of the peptide bond precedes (rather than follows) the carbonyl portion. See, generally, Goodman, M. and Chorev, M. Accounts of Chem. Res. 1979, 12, 423.

The reversed orientation peptides described herein include (a) those wherein one or more amino-terminal residues are converted to a reversed (“rev”) orientation (thus yielding a second “carboxyl terminus” at the left-most portion of the molecule), and (b) those wherein one or more carboxyl-terminal residues are converted to a reversed (“rev”) orientation (yielding a second “amino terminus” at the right-most portion of the molecule). A peptide (amide) bond cannot be formed at the interface between a normal orientation residue and a reverse orientation residue.

Therefore, certain reversed peptide compounds of the invention can be formed by utilizing an appropriate amino acid mimetic moiety to link the two adjacent portions of the sequences depicted above utilizing a reversed peptide (reversed amide) bond. In case (a) above, a central residue of a diketo compound may conveniently be utilized to link structures with two amide bonds to achieve a peptidomimetic structure. In case (b) above, a central residue of a diamino compound will likewise be useful to link structures with two amide bonds to form a peptidomimetic structure.

The reversed direction of bonding in such compounds will generally, in addition, require inversion of the enantiomeric configuration of the reversed amino acid residues in order to maintain a spatial orientation of side chains that is similar to that of the non-reversed peptide. The configuration of amino acids in the reversed portion of the peptides is preferably (D), and the configuration of the non-reversed portion is preferably (L). Opposite or mixed configurations are acceptable when appropriate to optimize a binding activity.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g., the ability to bind to the binding domain), wherein one or more simple variations of substituents are made which do not adversely affect the efficacy of the compound in binding to the binding domain. In general, the compounds of the present invention may be prepared by the methods illustrated in the general reaction schemes as, for example, described below, or by modifications thereof, using readily available starting materials, reagents and conventional synthesis procedures. Thus, the contemplated equivalents include peptidomimetic or non-peptide small molecule binders of the BMP-binding domain. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover. Also for purposes of this invention, the term “hydrocarbon” is contemplated to include all permissible compounds having at least one hydrogen and one carbon atom. In a broad aspect, the permissible hydrocarbons include acyclic and cyclic, branched and unbranched, carbocyclic and heterocyclic, aromatic and nonaromatic organic compounds which can be substituted or unsubstituted.

As used herein, the term “pharmaceutically acceptable” refers to a carrier medium which does not interfere with the effectiveness of the biological activity of the active ingredients and which is not excessively toxic to the hosts of the concentrations of which it is administered. The administration(s) may take place by any suitable technique, including subcutaneous and parenteral administration, preferably parenteral. Examples of parenteral administration include intravenous, intraarterial, intramuscular, and intraperitoneal, with intravenous being preferred.

As used herein, the term “prophylactic or therapeutic” treatment refers to administration to the host of the medical condition. If it is administered prior to exposure to the condition, the treatment is prophylactic (i.e., it protects the host against infection), whereas if administered after infection or initiation of the disease, the treatment is therapeutic (i.e., it combats the existing infection or cancer).

III. Structural Features of hOP-1 Monomer Subunits

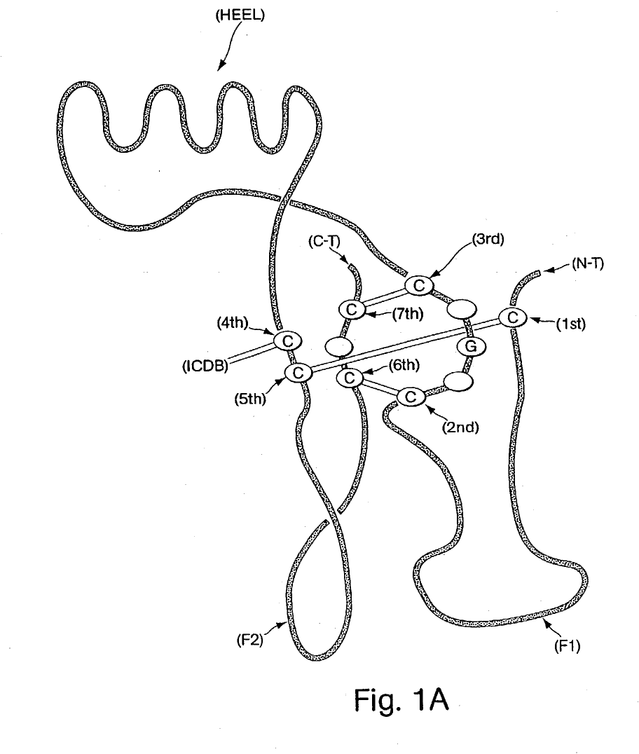

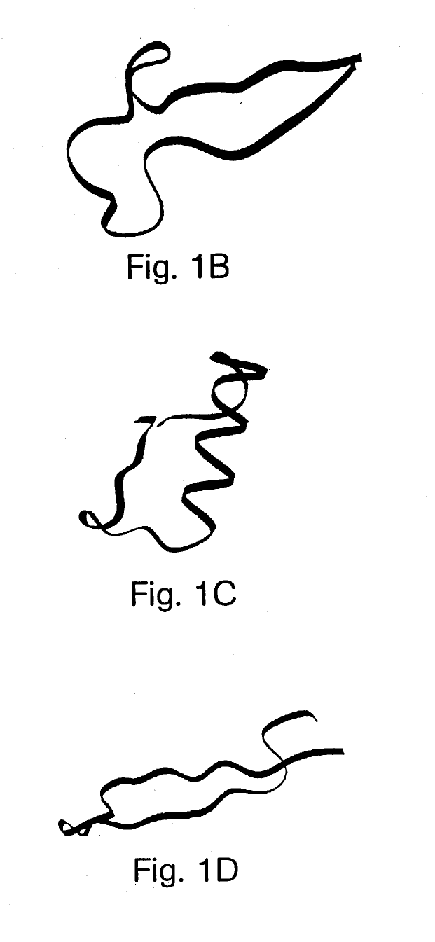

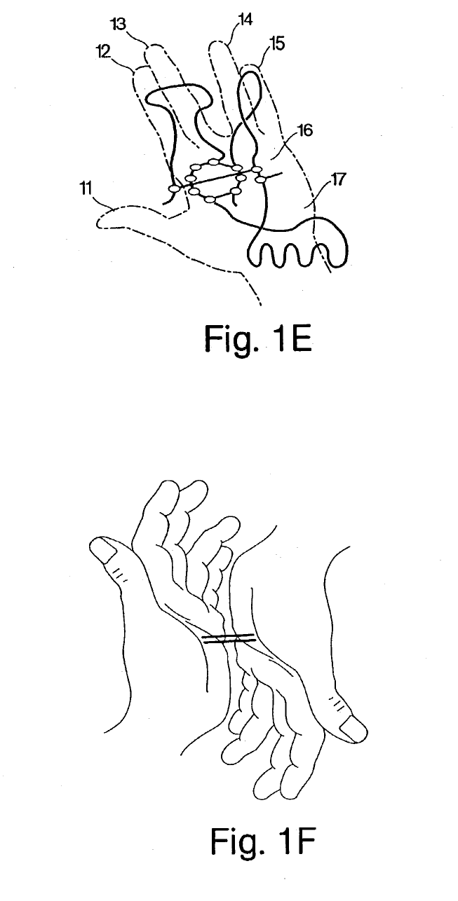

Human OP-1, like TGF-β2, is a dimeric protein having a unique folding pattern involving six of the seven C-terminal cysteine residues, as illustrated in FIG. 1A. Each of the subunits in OP-1, like TGF-β2 (See Daopin et al. (1992) Science 257:369-373; and Schulnegger et al. (1992) Nature 358:430-434) have a characteristic folding pattern, illustrated schematically in FIG. 1A, that involves six of the seven C-terminal cysteine residues.

Referring to FIG. 1A, four of the cysteine residues in each subunit form two disulfide bonds which together create an eight residue ring, while two additional cysteine residues form a disulfide bond that passes through the ring to form a knot-like structure (cysteine knot). With a numbering scheme beginning with the most N-terminal cysteine of the 7 conserved cysteine residues assigned number 1, the 2nd and 6th cysteine residues are disulfide bonded to close one side of the eight residue ring while the 3rd and 7th cysteine residues are disulfide bonded to close the other side of the ring. The 1st and 5th conserved cysteine residues are disulfide bonded through the center of the ring to form the core of the knot. Amino acid sequence alignment patterns suggest this structural motif is conserved between members of the TGF-β superfamily. The 4th cysteine is semi-conserved and when present typically forms an inter-chain disulfide bond (ICDB) with the corresponding cysteine residue in the other subunit.

Each hOP-1 monomer subunit comprises three major tertiary structural elements and an N-terminal region. The structural elements are made up of regions of contiguous polypeptide chain that possess over 50% secondary structure of the following types: (1) loop, (2) helix and (3) β-sheet. Furthermore, in these regions the N-terminal and C-terminal strands are not more than 7 Å apart.

The amino acid sequence between the 1st and 2nd conserved cysteines (FIG. 1A) form a structural region characterized by an anti-parallel β-sheet finger, referred to herein as the finger 1 region (F1). A ribbon trace of the human OP-1 finger 1 peptide backbone is shown in FIG. 1B. Similarly the residues between the 5th and 6th conserved cysteines in FIG. 1A also form an anti-parallel β-sheet finger, referred to herein as the finger 2 region (F2). A ribbon trace of the human OP-1 finger 2 peptide backbone is shown in FIG. 1D. A β-sheet finger is a single amino acid chain, comprising a β-strand that folds back on itself by means of a β-turn or some larger loop so that the entering and exiting strands form one or more anti-parallel β-sheet structures. The third major structural region, involving the residues between the 3rd and 4th conserved cysteines in FIG. 1A, is characterized by a three-turn a-helix referred to herein as the heel region (H). A ribbon trace of the human OP-1 heel peptide backbone is shown in FIG. 1C.

The organization of the monomer structure is similar to that of a left hand (see FIG. 1E) where the knot region is located at the position equivalent to the palm (16), the finger 1 region is equivalent to the index and middle fingers (12 and 13, respectively), the α-helix, or heel region, is equivalent to the heel of the hand (17), and the finger 2 region is equivalent to the ring and small fingers (14 and 15, respectively). The N-terminal region is predicted to be located at a position roughly equivalent to the thumb (11).

Monovision ribbon tracings illustrating the alpha carbon backbones of each of the three major independent structural elements of the monomer are illustrated in FIGS. 1B-1D. Specifically, the finger 1 region comprising the first anti-parallel β-sheet segment is shown in FIG. 1B, the heel region comprising the three turn a-helical segment is shown in FIG. 1C, and the finger 2 region comprising second and third anti-parallel β-sheet segments is shown in FIG. 1D.

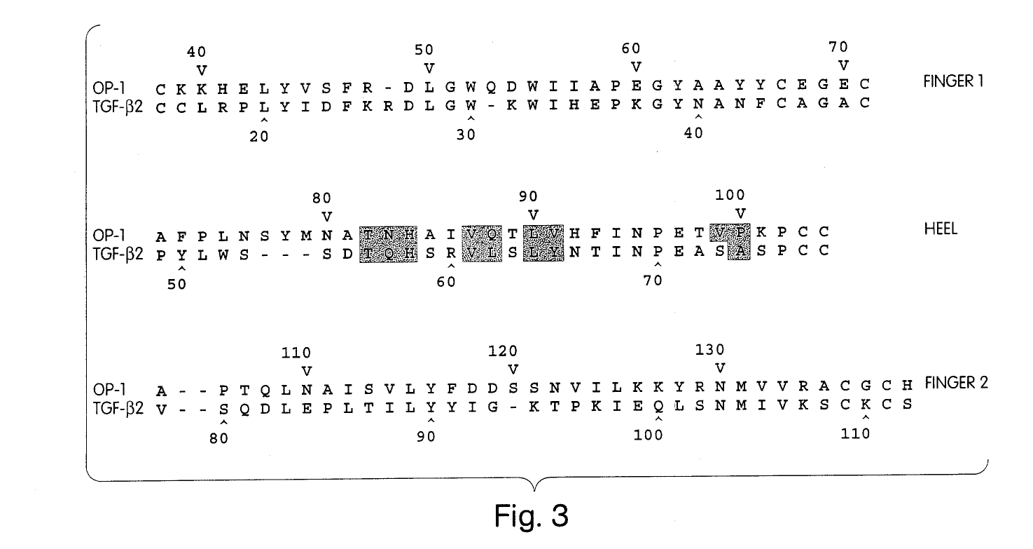

For the sake of comparison, FIG. 3 shows an alignment of the amino acid sequences defining the finger 1, finger 2 and heel regions of hOP-1 and TGF-β2. In FIG. 3, the OP-1 and TGF-β2 amino acid sequences were aligned according to the corresponding regions of local structural identity in the OP-1 and TGF-β2 structures. Alignment gaps were positioned in loop regions, which is where the local conformational homology of the α-carbon traces tends to be the lowest.

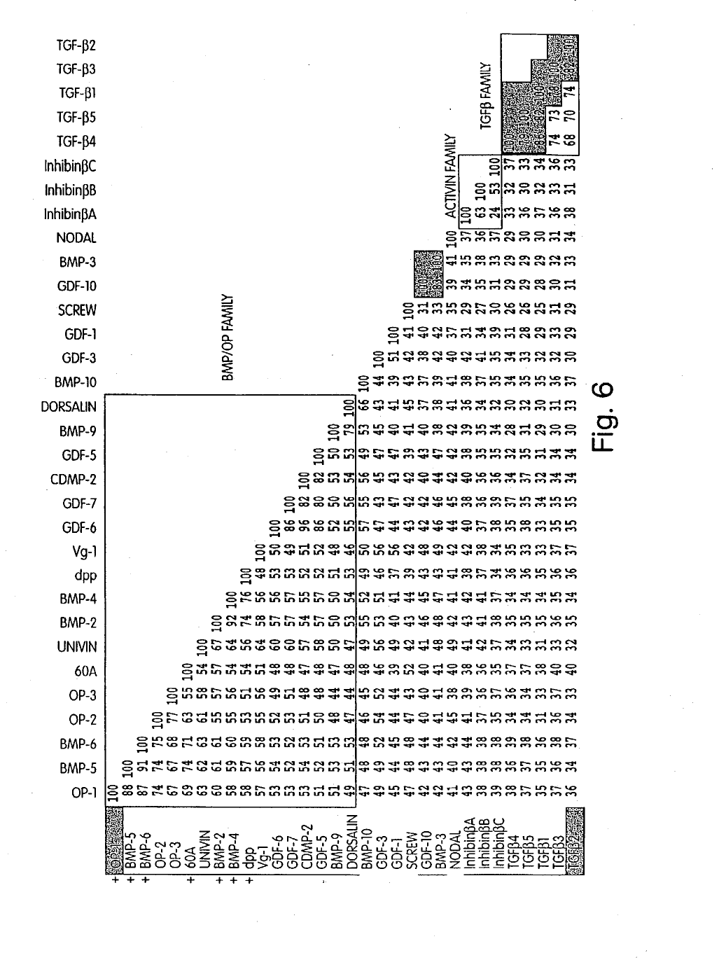

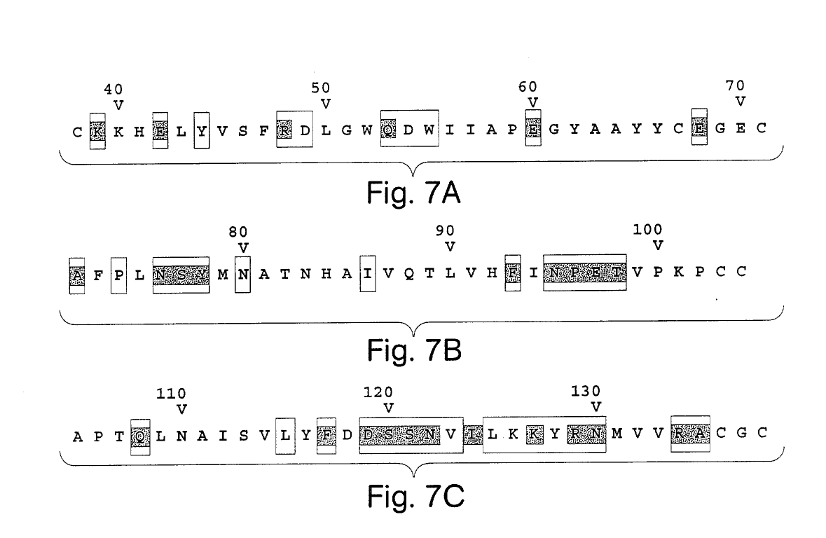

The structure-based alignment of OP-1 and TGF-β2 then was used as a template for the alignment of the 7-cysteine domain sequences of other TGF-β superfamily members (other members of the TGF-β superfamily are set forth in FIG. 6). Alignment gaps were positioned in regions which are loops in both the OP-1 and TGF-β2 structures. Percent identity between pairs of sequences was calculated as the number of identical aligned sequence positions, excluding gaps, normalized to the geometric mean of the lengths of the sequences and multiplied by 100. FIG. 6 is a matrix of the resulting pair wise present identities between super family sequences so aligned. Using such principles, it is contemplated that the hOP-1 and TGF-β structures, either alone or in combination, may be used for homology modeling of other proteins belonging to the TGF-β superfamily whose three-dimensional structures have not yet been determined (see, for example, the other members of the TGF-β superfamily listed in FIG. 6). It is contemplated that such models may be useful in designing morphogen analogs for the particular candidate morphogens of interest, however, for simplicity, the disclosure hereinbelow refers specifically the design, identification, and production of morphogen analogs of hOP-1.

FIG. 3 also shows, based on an analysis of the 2.8 Å resolution structure, a comparison of interchain contact residues in OP-1 and TGF-β2. Residues were designated as contact residues if the distance between the centers of at least one non-hydrogen atom from each side chain was less than the sum of their Van der Waals radii plus 1.1 Å. Despite the low level of sequence identity between OP-1 and TGF-β2, the inter chain contacts between residues in the heel of one chain and residues in finger 1 and finger 2 of the other chain are well conserved.

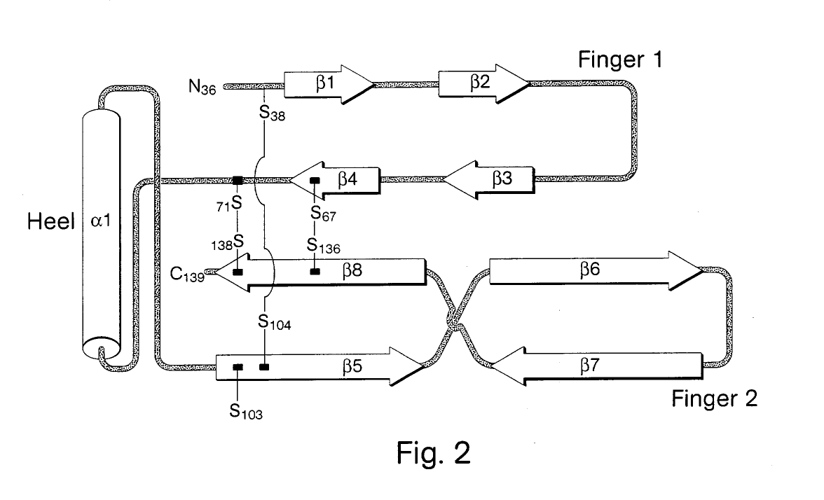

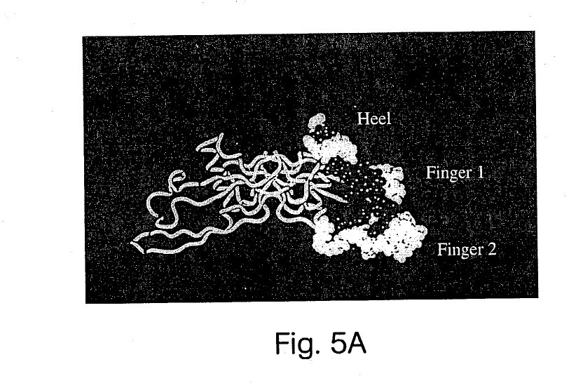

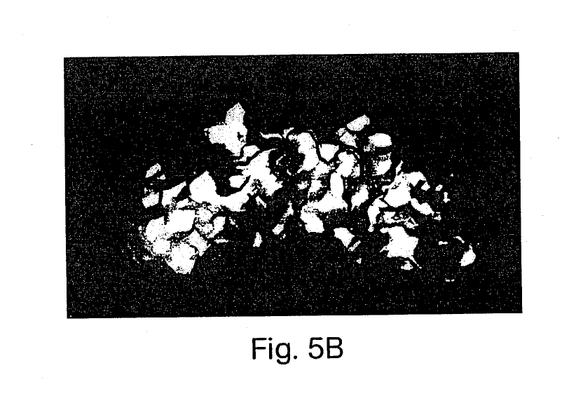

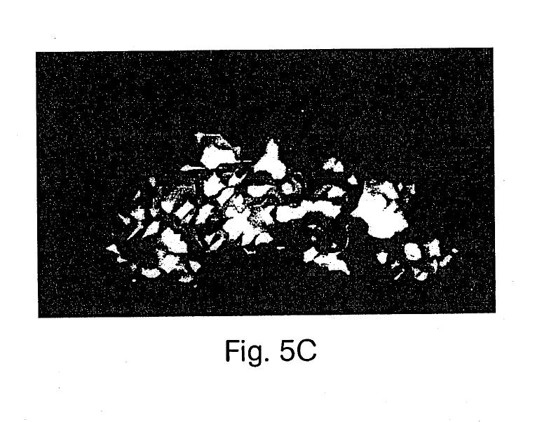

Upon detailed inspection of the 2.8 Å resolution structure of hOP-1, the finger 1 region of hOP-1 is an antiparallel β-sheet containing a thirteen residue omega loop (Phe47-Glu60) (FIG. 2). The structural alignment of the OP-1 and TGF-β 2 sequences in FIG. 3 places two gaps in the omega loop. The first gap represents a deletion in hOP-1 that aligns with Arg26 in the α2 helix of TGF-β2. This deletion results in a tighter, non-(a-helical turn in OP-1 as compared with TGF-β2. The second gap corresponds to the insertion of Gln53 in OP-1, which has the result of directing both Gln53 and Asp54 side chains into the solvent. By comparison, in the corresponding region of TGF-β2, only Lys31 is in contact with the solvent. These differences in the conformation of the omega loop also result in the conserved proline (Pro59) adopting a trans conformation in hOP-1 rather than cis, as in TGF-β2. The conformation of the omega loop orients six non-polar residues so they can contribute to a solvent inaccessible interface with Finger 2. Of these six, four are aromatic (Phe47, Trp55, Tyr62 and Tyr65), and two are aliphatic (Ile56 and Ile57). In all, the conformation of the omega loop backbone places five polar residues (Arg48, Asp49, Gln53, Asp54, and Glu60) in contact with solvent. The net surface charge in this region is −2 whereas it is +2 for TGF-β2 (FIG. 5).

According to the 2.8 Å structure, the only a helix in the monomer is located between the third and fifth cysteines (Cys71 and Cys104). This helix extends for three and one-half turns from residues Thr82 to Ile94, is amphipathic, and contains a number of hydrophobic residues which in the dimer make contact with residues from Finger 1 and Finger 2 of the other monomer (FIG. 3). Several hydrophilic residues (Thr82, His84, and Gln88) form one wall of an internal solvent pocket near the 2-fold axis of the dimer, while others (Asn83, His92, and Asn95) are in contact with the external solvent. The conformation of the loop leading from the C-terminal end of the helix back to the cysteine knot is similar in OP-1 and TGF-β2. By comparison, the loop located at the N-terminal end of the helix is 3 residues longer in OP-1, resulting in a different fold than in TGF-β2. In this loop of OP-1, it is believed that an N-linked sugar moiety is attached to Asn80, however, no such corresponding glycosylation site exists in TGF-β2. Further, this loop is uncharged in OP-1 whereas it is negatively charged in TGF-β2.

According to the 2.8 Å structure, Finger 2 is the second antiparallel β-sheet in OP-1 (FIG. 2). The polypeptide chain reverses direction between segments β6 and β7 through a 3:5 turn (Sibanda, et al. (1991) Methods in Enzymol. 202:59-82) beginning at residue Asp118 and ending at residue Asn122. In contrast, TGF-β2 has one less residue in this loop and adopts a 2:2 turn (Sibanda et al. (1991) supra). Residues Arg129 to Val132, located between segments β7 and β8, form a peptide bridge that crosses over the C-terminal end of strand β5 and produces a 180° twist in the Finger 2 antiparallel β-structure. A similar structure is observed in other cysteine knot growth factors, however the peptide bridge length varies (McDonald et al. (1991) Nature 354:411-414). Within the monomer, Finger 2 makes intra-chain contacts with Finger 1 by contributing aromatic residues Tyr116, Phe117 and Tyr128, and aliphatic residues Val 114, Leu115, Val123, Met131 and Val133 to a solvent inaccessible interface. OP-1 and TGF-β2 differ by three charges in the region of the Finger 2 turn; OP-1 has two negative charges while TGF-β2 has one positive charge. In the region between the turn and the peptide bridge, OP-1 has a net charge of +3 while TGF-β2 is neutral (FIG. 5).

The N-terminus of each monomeric subunit is believed to be highly mobile and has not been resolved in the 2.8 Å resolution structure of hOP-1. The N-terminal region can be deleted without adversely affecting biological activity and, therefore, it is contemplated that this portion of mature hOP-1 may be removed and replaced with other protein or peptide sequences, such as antibodies, and/or radiolabel binding sites for enhancing targeting to a particular locus in vivo or for use in in vivo imaging experiments. In addition, the N-terminal region may be replaced with an ion chelating motif (e.g., His₆) for use in affinity purification schemes, or replaced with proteins or peptides for enhancing solubility in aqueous solvents.

There is significant sequence and structural conservation among members of the TGFβ superfamily. Notably, the important structural domains of the mature OP-1 protein outlined in detail above (finger-1, heel, and finger-2) are also present in other morphogenic proteins of the TGFβ superfamily. Accordingly, peptides comprising all or a portion of these structural motifs will likely agonize or antagonize the function of that morphogenic protein. Exemplary morphogenic proteins related to OP-1 include BMP5, BMP6, BMP2, BMP4, BMP8/OP2, GDF1, GDF5, GDF6, and GDF7.

The sequences of these and other morphogenic proteins are well known in the art, and are provided herein to facilitate reference to specific structural domains. The full-length amino acid sequences for BMP5, BMP6, BMP2, BMP4, BMP8/OP2, GDF1, GDF5, and GDF6 precursor proteins are provided. One skilled in the art will recognize that the mature, functional morphogenic protein which contains the characteristic finger-1, heel, and finger-2 regions arises from the C-terminal portion of the precursor protein. For clarity, the position of these regions will be given with respect to their position in the context of the full length protein. The sequence of GDF7 provided herein does not correspond to the full length precursor protein, but rather to a C-terminal fragment. The position of characteristic domains of GDF7 is given with respect to their position within this fragment.

The full length amino acid sequence of BMP5 is well known in the art (see GenBank Accession Numbers: P22003 and NP_{—}066551) and provided herein (SEQ ID NO: 29). Finger-1 corresponds to Cys353-Cys386 (SEQ ID NO: 30), the heel region corresponds to Ser387-Cys419 (SEQ ID NO: 31), and finger-2 corresponds to Ala420-Cys453 (SEQ ID NO: 32).

The full length amino acid sequence of BMP6 is well known in the art (see GenBank Accession Numbers: P22004 and NP_{—}001709) and provided herein (SEQ ID NO: 33). Finger-1 corresponds to Cys412-Cys445 (SEQ ID NO: 34), the heel region corresponds to Ser446-Cys478 (SEQ ID NO: 35), and finger-2 corresponds to Ala479-Cys52 (SEQ ID NO: 36).

The full length amino acid sequence of BMP2 is well known in the art (see GenBank Accession Numbers: P12643 and NP_{—}001191) and provided herein (SEQ ID NO: 37). Finger-1 corresponds to Cys296-Cys329 (SEQ ID NO: 38), the heel region corresponds to Pro330-Cys361 (SEQ ID NO: 39), and finger-2 corresponds to Val362-Cys395 (SEQ ID NO: 40).

The full length amino acid sequence of BMP4 is well known in the art (see GenBank Accession Numbers: P12644 and NP_{—}001193) and provided herein (SEQ ID NO: 41). Finger-1 corresponds to Cys308-Cys341 (SEQ ID NO: 42), the heel region corresponds to Pro342-Cys373 (SEQ ID NO: 43), and finger-2 corresponds to Val374-Cys407 (SEQ ID NO: 44).

The full length amino acid sequence of BMP8/OP2 is well known in the art (see GenBank Accession Numbers: P34820 and NP_{—}001711) and provided herein (SEQ ID NO: 45). Finger-1 corresponds to Cys301-Cys334 (SEQ ID NO: 46), the heel region corresponds to Ser335-Cys367 (SEQ ID NO: 47), and finger-2 corresponds to Ala368-Cys401 (SEQ ID NO: 48).

The full length amino acid sequence of GDF1 is well known in the art (see GenBank Accession Numbers: P27539 and NP_{—}001483) and provided herein (SEQ ID NO: 49). Finger-1 corresponds to Cys267-Cys30O (SEQ ID NO: 50), the heel region corresponds to Ala301-Cys337 (SEQ ID NO: 51), and finger-2 corresponds to Val338-Cys371 (SEQ ID NO: 52).

The full length amino acid sequence of GDF5 is well known in the art (see GenBank Accession Numbers: P43026 and NP_{—}000548) and provided herein (SEQ ID NO: 53). Finger-1 corresponds to Cys400-Cys433 (SEQ ID NO: 54), the heel region corresponds to Glu434-Cys466 (SEQ ID NO: 55), and finger-2 corresponds to Val467-Cys500 (SEQ ID NO: 56).

The full length amino acid sequence of mammalian GDF6 is well known in the art (see GenBank Accession Numbers: P55106 for bovine GDF6) and provided herein (SEQ ID NO: 57). Finger-1 corresponds to Cys335-Cys368 (SEQ ID NO: 58), the heel region corresponds to Asp369-Cys401 (SEQ ID NO: 59), and finger-2 corresponds to Val402-Cys435 (SEQ ID NO: 60).

The amino acid sequence of mammalian GDF7 is well known in the art (see GenBank Accession Numbers: AAA18780 for a fragment of murine GDF7) and provided herein (SEQ ID NO: 61). Finger-1 corresponds to Cys50-Cys83 (SEQ ID NO: 62), the heel region corresponds to Asp84-Cys115 (SEQ ID NO: 63), and finger-2 corresponds to Val116-Cys150 (SEQ ID NO: 64).

IV. Structural Features of the hOP-1 Dimer

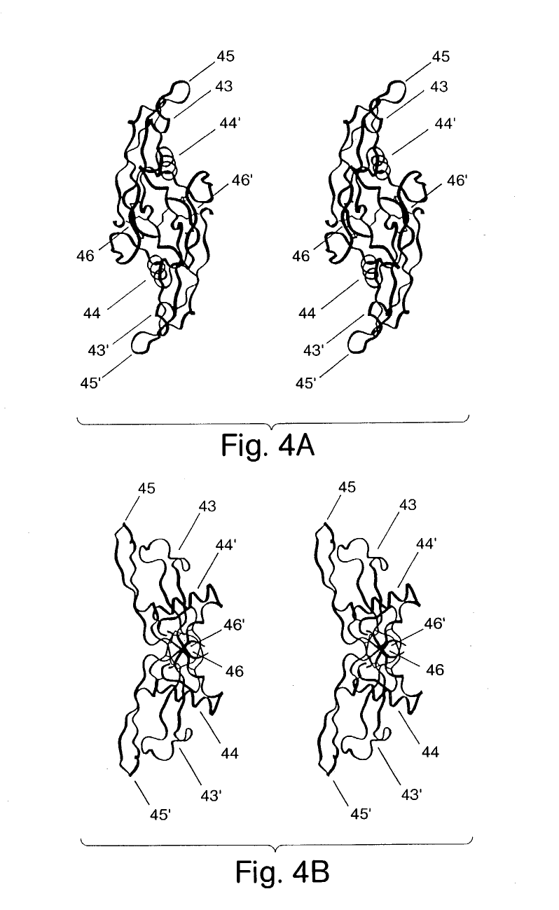

FIG. 4 shows stereo ribbon trace drawings representative of the peptide backbone of the hOP-1 dimer complex, based on the 2.8 Å structure. The two monomer subunits in the dimer complex are oriented symmetrically such that the heel region of one subunit contacts the finger regions of the other subunit with the knot regions of the connected subunits forming the core of the molecule. The 4th cysteine forms an inter-chain disulfide bond with its counterpart on the second chain thereby equivalently linking the chains at the center of the palms. The dimer thus formed is an ellipsoidal (cigar shaped) molecule when viewed from the top looking down the two-fold axis of symmetry between the subunits (FIG. 4A). Viewed from the side, the molecule resembles a bent “cigar” since the two subunits are oriented at a slight angle relative to each other (FIG. 4B).

As shown in FIG. 4, each of the structural elements which together define the native monomer subunits of the dimer are labeled 43, 43′, 44, 44′, 45, 45′, 46, and 46′, wherein, elements 43, 44, 45, and 46 are defined by one subunit and elements 43′, 44′, 45′, and 46′ belong to the other subunit. Specifically, 43 and 43′ denote the finger 1 regions; 44 and 44′ denote heel regions; 45 and 45′ denote the finger 2 regions; and 46 and 46′ denote disulfide bonds which connect the 1st and 5th conserved cysteines of each subunit to form the knot-like structure. From FIG. 4, it can be seen that the heel region from one subunit, e.g., 44, and the finger 1 and finger 2 regions, e.g., 43′ and 45′, respectively from the other subunit, interact with one another. These three elements are believed to cooperate with one another to define a structure interactive with the ligand binding interactive surface of the cognate receptor.

The helical axis is defined as the line equidistant from the alpha carbons in the helical region. A sequence of four points is needed to define the dihedral angle between the axes of the helices in the dimer. The two inner points were chosen to lie on the helical axes adjacent to the a-carbon of residue His 84 in OP-1 or His 58 in TGF-β2, respectively. The two outer points were chosen to lie on their respective helical axes, but their location is arbitrary. To measure the angle between the helices, the first two points used to define the dihedral angle were translated so as to superimpose the inner points. The resulting three points define the angle.

A major difference between the OP-1 and TGF-β2 dimers is the relative orientation of the helices in the heel region. The angle between the axes of the helices in the heel region of OP-1 is 43° which is 10° larger than that measured for TGF-β2. The measured dihedral angle between the helices is −20° for OP-1 which is 14° more negative than for TGF-β2. Despite these differences in helical orientation, the same helix and finger residue positions are involved in making inter-chain contacts, as evidenced by the shaded residues in FIG. 3.

Solution Electrostatic Potentials on the Surface of OP-1 and TGF-β2

The solution electrostatic potentials surrounding the OP-1 and TGF-β2 (1TFG) (Schlunegger et al (1992) supra) dimers were calculated using DELPHI (Gilson et al. (1987) Nature 330:84-86; and Nicholls et al. (1991) J. Comput. Chem. 12:435-445) (Biosym Technologies, Inc., San Diego, Calif.). The calculations were performed using a solvent dielectric constant of 80, a solvent radius of 1.4 Å, an ionic strength of 0.145 M and an ionic radius of 2.0 Å. The interior of the protein was modeled using a dielectric constant of 2.0. Formal charges were used and distributed as follows: atoms OD1 and OD2 of Asp were each charged −0.5, atoms OE1 and OE2 of Glu were each charged −0.5, atoms NG1 and NE2 of His were each charged 0.25, atom NZ of Lys was charged +1.0, atoms NH1 and NH2 of Arg were each charged +0.5, and atom OXT of the C-terminal carboxyl group was charged −1.0.

The differences in charge distribution on the surfaces of OP-1 and TGF-β2 can be observed by comparing the color distributions of FIGS. 5B and 5C, respectively. Surface regions having an electrostatic potential of −3 kT or less are shown in red while surface regions of +3 kT or greater are shown in blue. Neutral regions are shown in green or gold to correspond to the backbone ribbons shown in FIG. 5A. As mentioned in the following section, the differences in electrostatic potential on the surfaces of OP-1 and TGF-β2 may play an important role in the specific interactions of the TGF-β superfamily members with their cognate receptors.

Receptor-Binding Domain

Without wishing to be bound by theory, it is contemplated that the receptor-binding regions of hOP-1 includes amino acids that are both solvent accessible and lie at positions of heterogeneous composition, as determined from the amino acid sequence of hOP-1 when aligned with other members of the TGF-β superfamily (See FIG. 3).

Divergent structural features in hOP-1, like TGF-β2, occur primarily in the external loops of finger 1 and finger 2, the loops bordering the helix in the heel region, and the residues in the N-terminal domain preceding the first cysteine of the cysteine knot.

These regions are solvent accessible. In both the OP-1 and TGF-β2 dimer structures, the tip of finger 2 and the omega loop of finger 1 from one chain, and the C-terminal end of the α-helix in the heel of the other chain form a contiguous ridge approximately 40 Å long and 15 Å wide (FIG. 5A). It is contemplated that this ridge contains the primary structural features that interact with the cognate receptor, and that the binding specificity between different TGF-β superfamily members derives from conformational and electrostatic variations on the surface of this ridge.

Differences in the conformation of the finger 1 omega loop, which constitutes the mid section of the ridge, and in the turn at the end of finger 2, which forms one end of the ridge are noted. However, there are striking differences in the surface charge of the ridge in hOP-1 relative to TGF-β2 (see FIGS. 5B and 5C). In hOP-1, the ends of the finger regions are negatively charged whereas in TGF-β2, the ends of the finger regions are positively charged. This results in a net charge of −4 for the receptor-binding ridge of hOP-1 versus +3 for TGF-β2. Conversely, the N-strand located C-terminal to the turn of finger 2 (β7, FIG. 2) is positively charged in OP-1 whereas it is negatively charged in TGF-β2 (FIGS. 5B and 5C). These features suggest that electrostatic charge distribution plays an important role in the specific interactions of the TGF-β superfamily members with their cognate receptors.

V. Design of Morphogen Analogs

A. Engineering Small Molecules Based upon the hOP-1 Structure

The availability of atomic coordinates for hOP-1 enables the skilled artisan to design small molecules, for example, peptides or non-peptidyl based organic molecules having certain chemical features, which mimic the biological activity of a BMP, such as hOP-1. Chemical features of interest may include, for example, the three-dimensional structure of a particular protein domain, solvent accessible surface of a particular protein domain, spatial distribution of charged and/or hydrophobic chemical moieties, electrostatic charge distribution, or a combination thereof. Such chemical features may readily be determined from the three-dimensional representation of hOP-1.

Furthermore, given the high level of sequence and structural conservation among members of the TGF-β superfamily, morphogen analogs corresponding to the conserved finger-1, heel, and finger-2 regions of other morphogenic proteins can be designed by one skilled in the art. Such morphogen analogs can be readily tested, using the methods disclosed herein for the testing of OP-1 analogs, for the ability to mimic the biological activity of a TGF-β superfamily member. Exemplary TGF-β superfamily members amenable to this approach include BMP2, BMP4, BMP5, BMP6, OP2, OP3, GDF1, GDF5, GDF6, and GDF7, and sequences corresponding to the finger-1, heel, and finger-2 regions of each of these proteins are described herein.

(i) Peptides

After having determined which amino acid residues contribute to the receptor-binding domain (supra), it is possible for the skilled artisan to design synthetic peptides having amino acid sequences that define a pre-selected receptor-binding motif. A computer program useful in designing potentially bioactive peptidomimetics is described in U.S. Pat. No. 5,331,573, the disclosure of which is incorporated by reference herein.

In addition to choosing a desirable amino acid sequence, the skilled artisan using standard molecular modeling software packages, infra, can design specific peptides having, for example, additional cysteine amino acids located at pre-selected positions to facilitate cyclization of the peptide of interest. Oxidation of the additional cysteine residues results in cyclization of the peptide thereby constraining the peptide in a conformation that mimics the conformation of the corresponding amino acid sequence in native hOP-1. It is contemplated, that any standard covalent linkage, for example, disulfide bonds, typically used to cyclize synthetic peptides maybe useful in the practice of the instant invention. Alternative cyclization chemistries are discussed in International Application PCT/WO 95/01800, the disclosure of which is incorporated herein by reference.

In addition, it is contemplated that a single peptide containing amino acid sequences derived from separate hOP-1 subunit domains, for example, a single peptide having an amino acid sequence defining the tip of the finger 1 region linked by means of a polypeptide linker to an amino acid sequence defining the tip of the finger 2 region. The amino sequence defining each of the finger regions may further comprise a means, for example, disulfide bonds for cyclizing each finger region motif. The resulting peptide therefore comprises a single polypeptide chain having a first amino acid sequence defining a three-dimensional domain mimicking the tip of the finger 1 region and a second said sequence defining a three-dimensional domain mimicking the tip of the finger 2 region.

BMP analogs, including peptides, peptidomimetics, non-peptide small molecules, genes and recombinant polypeptides, may be generated using combinatorial techniques using techniques which are available in the art for generating combinatorial libraries of small organic/peptide libraries. See, for example, Blondelle et al. (1995) Trends Anal. Chem. 14:83; the Affymax U.S. Pat. Nos. 5,359,115 and 5,362,899; the Ellman U.S. Pat. No. 5,288,514; the Still et al. PCT publication WO 94/08051; Chen et al. (1994) JACS 116:2661; Kerr et al. (1993) JACS 115:252; PCT publications WO92/10092, WO93/09668 and WO91/07087; and the Lerner et al. PCT publication WO93/20242).

In a preferred embodiment, the combinatorial peptide library is produced by way of a degenerate library of genes encoding a library of polypeptides which each include at least a portion of potential BMP analog peptide sequences. For instance, a mixture of synthetic oligonucleotides can be enzymatically ligated into gene sequences such that the degenerate set of potential OP-1 analog nucleotide sequences are expressible as individual polypeptides, or alternatively, as a set of larger fusion proteins (e.g., for phage display) containing the set of BMP analog peptide sequences therein.

There are many ways by which the gene library of potential BMP homologs can be generated from a degenerate oligonucleotide sequence. Chemical synthesis of a degenerate gene sequence can be carried out in an automatic DNA synthesizer, and the synthetic genes can then be ligated into an appropriate gene for expression. The purpose of a degenerate set of genes is to provide, in one mixture, all of the sequences encoding the desired set of potential BMP analog sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp. 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

A wide range of techniques are known in the art for screening gene products of combinatorial libraries made by point mutations. Such techniques will be generally adaptable for rapid screening of the gene libraries generated by the combinatorial mutagenesis of BMP analog sequences. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Such illustrative assays are amenable to high throughput analysis as necessary to screen large numbers of degenerate sequences created by combinatorial mutagenesis techniques.

In yet another screening assay, the candidate peptides are displayed on the surface of a cell or viral particle, and the ability of particular cells or viral particles to associate with a BMP/OP receptor via this gene product is detected in a “panning assay”. Such panning steps can be carried out on cells cultured from embryos. For instance, the gene library can be cloned into the gene for a surface membrane protein of a bacterial cell, and the resulting fusion protein detected by panning (Ladner et al., WO 88/06630; Fuchs et al. (1991) Bio/technology 9:1370-1371; and Goward et al. (1992) TIBS 18:136-140). In a similar fashion, fluorescently labeled molecules which bind a BMP/OP protein can be used to score for potentially functional peptides. Cells can be visually inspected and separated under a fluorescence microscope, or, where the morphology of the cell permits, separated by a fluorescence-activated cell sorter.

In an alternate embodiment, the gene library is expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at very high concentrations, large numbers of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and f1 are most often used in phage display libraries, as either of the phage gIII or gVIII coat proteins can be used to generate fusion proteins without disrupting the ultimate packaging of the viral particle (Ladner et al. PCT publication WO 90/02909; Garrard et al., PCT publication WO 92/09690; Marks et al. (1992) J. Biol. Chem. 267:16007-16010; Griffths et al. (1993) EMBO J 12:725-734; Clackson et al. (1991) Nature 352:624-628; and Barbas et al. (1992) PNAS 89:4457-4461).

In an illustrative embodiment, the recombinant phage antibody system (RPAS, Pharamacia Catalog number 27-9400-01) can be easily modified for use in expressing and screening peptide combinatorial libraries. For instance, the pCANTAB 5 phagemid of the RPAS kit contains the gene which encodes the phage gIII coat protein. The peptide combinatorial gene library can be cloned into the phagemid adjacent to the gIII signal sequence such that it will be expressed as a gIII fusion protein. After ligation, the phagemid is used to transform competent E. coli TG1 cells. Transformed cells are subsequently infected with M13KO7 helper phage to rescue the phagemid and its candidate peptide gene insert. The resulting recombinant phage contain phagemid DNA encoding a specific candidate peptide, and display one or more copies of the corresponding fusion coat protein. The phage-displayed candidate peptides which are capable of binding a BMP/OP receptor are selected or enriched by panning. For instance, the phage library can be applied to cells which express a BMP/OP receptor and unbound phage washed away from the cells. The bound phage are then isolated, and if the recombinant phage express at least one copy of the wild type gIII coat protein, they will retain their ability to infect E. coli. Thus, successive rounds of reinfection of E. coli, and panning will greatly enrich for BMP/OP homologs, which can then be screened for further biological activities in order to differentiate agonists and antagonists.

These techniques have been successfully employed in the art to identify peptide mimetics. For example, potent agonists of erythropoietin (Epo) were identified using random phage display to isolate small peptides which bind to and activate the Epo receptor (Wrighton et al. (1996) Science 273: 458). More recently, Kaushansky has reviewed a large body of work wherein both peptide and small molecule mimetics were identified using several different experimental approaches (Kaushansky (2001) Annals of the NY Academy of Sciences 938: 131).

Combinatorial mutagenesis has a potential to generate very large libraries of different peptides, e.g., in the order of 10²⁶ molecules. Combinatorial libraries of this size may be technically challenging to screen even with high-throughput screening assays such as phage display. To overcome this problem, a new technique has been developed recently, recursive ensemble mutagenesis (REM), which allows one to avoid the very high proportion of non-functional proteins in a random library and simply enhances the frequency of functional proteins, thus decreasing the complexity required to achieve a useful sampling of sequence space. REM is an algorithm which enhances the frequency of functional mutants in a library when an appropriate selection or screening method is employed (Arkin and Yourvan, 1992, PNAS USA 89:7811-7815; Yourvan et al., 1992, Parallel Problem Solving from Nature, 2., In Maenner and Manderick, eds., Elsevir Publishing Co., Amsterdam, pp. 401-410; Delgrave et al., 1993, Protein Engineering 6(3):327-331).

Recombinantly produced forms of the subject peptides can be produced using, for example, expression vectors containing a nucleic acid encoding a subject peptide, operably linked to at least one transcriptional regulatory sequence. Operably linked is intended to mean that the nucleotide sequence is linked to a regulatory sequence in a manner which allows expression of the nucleotide sequence. Regulatory sequences are art-recognized and are selected to direct expression of a subject polypeptide. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences, sequences that control the expression of a DNA sequence when operatively linked to it, may be used in these vectors to express DNA sequences encoding subject polypeptide. Such useful expression control sequences, include, for example, a viral LTR, such as the LTR of the Moloney murine leukemia virus, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage λ, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

Such peptides may be synthesized and screened for BMP-like activity using any of the standard protocols described below.

(ii) Organic Molecules

As discussed above, upon determination of the receptor-binding domain of hOP-1, it is contemplated that the skilled artisan can design non-peptidyl based small molecules, for example, small organic molecules, whose structural and chemical features mimic the same features displayed on at least part of the surface of the receptor-binding domain of hOP-1.

Because a major contribution to the receptor-binding surface is the spatial arrangement of chemically interactive moieties present within the sidechains of amino acids which together define the receptor-binding surface, a preferred embodiment of the present invention relates to designing and producing a synthetic organic molecule having a framework that carries chemically interactive moieties in a spatial relationship that mimics the spatial relationship of the chemical moieties disposed on the amino acid sidechains which constitute the receptor-binding site of hOP-1. Preferred chemical moieties, include but are not limited to, the chemical moieties defined by the amino acid side chains of amino acids believed to constitute the receptor-binding domain of hOP-1. It is understood, therefore, that the receptor-binding surface of the morphogen analog need not comprise amino acid residues but the chemical moieties disposed thereon.

For example, upon identification of relevant chemical groups, the skilled artisan using a conventional computer program can design a small molecule having the receptor interactive chemical moieties disposed upon a suitable carrier framework. Useful computer programs are described in, for example, Dixon (1992) Tibtech 10: 357-363; Tschinke et al. (1993) J. Med. Chem 36: 3863-3870; and Eisen el al. (1994) Proteins: Structure, Function, and Genetics 19: 199-221, the disclosures of which are incorporated herein by reference.

One particular computer program entitled “CAVEAT” searches a database, for example, the Cambridge Structural Database, for structures which have desired spatial orientations of chemical moieties (Bartlett et al. (1989) in “Molecular Recognition: Chemical and Biological Problems” (Roberts, S. M., ed.) pp. 182-196). The CAVEAT program has been used to design analogs of tendamistat, a 74 residue inhibitor of a-amylase, based on the orientation of selected amino acid side chains in the three-dimensional structure of tendamistat (Bartlett et al. (1989) supra).

Alternatively, upon identification of a series of analogs which mimic the biological activity of a BMP, such as OP-1, as determined by in vivo or in vitro assays, the skilled artisan may use a variety of computer programs which assist the skilled artisan to develop quantitative structure activity relationships (QSAR) and further to assist in the de novo design of additional morphogen analogs. Other useful computer programs are described in, for example, Connolly Martin (1991) Methods in Enzymology 203:587-613; Dixon (1992) supra; and Waszkowycz et al. (1994) J. Med. Chenm. 37: 3994-4002.

Thus, for example, one can begin with a portion of the three dimensional structure of a BMP, such as OP-1 (or a related morphogen), corresponding to a region of known or suspected biological importance. One such region is the solvent accessible loop or “tip” of the finger 2 region between the β6 and β7 sheets (i.e., from approximately residues 118-122). Synthetic, cyclic peptides (i.e., F2-2 and F2-3) were produced including this region (and several flanking residues) and were shown to possess OP-1-like biological activity (see Examples below). Based upon the three-dimensional structure of this region, disclosed herein, one is now enabled to produce more effective OP-1-like (or, generally, morphogen-like) analogs. For example, the charged carboxy groups of Asp 118 and Asp 119, and the relatively hydrophilic hydroxyl groups of Ser 120 and Ser 121, are solvent accessible and believed to be involved in OP-1 receptor binding. These functional groups define a contiguous portion of the three dimensional structure of the OP-1 surface. The peptide backbone of these residues, however, is not solvent accessible and, therefore, is not believed to form a portion of the three-dimensional surface of the OP-1 molecule. Thus, one of ordinary skill in the art, when choosing or designing a BMP, such as OP-1, or morphogen analog, can choose or design a molecule having the same or substantially equivalent (e.g., thiol v. hydroxyl) functional groups in substantially the same (e.g., ±1-3 Å) three-dimensional conformation. The same is true for other regions of interest in the OP-1 monomers or dimers (e.g., the receptor-binding domain, the finger 1, finger 2, or heel regions, or solvent accessible portions thereof). By using the three-dimensional structures disclosed herein, including the disclosure of the positions of solvent accessible and probable receptor contact residues, one of ordinary skill in the art can choose a portion of the three-dimensional structure of the OP-1 (or a related morphogen) molecule and, using this “portion” as a template select or design an analog which functionally mimics the template structure.

The molecular framework or backbone of the morphogen analog can be freely chosen by one of ordinary skill in the art so that it (1) joins the functional groups which mimic the portion of the morphogen's contiguous three-dimensional surface, including charge distribution and hydrophobicity/hydrophilicity characteristics, and (2) maintains or, at least, allows the functional groups to maintain the appropriate three-dimensional surface interaction and spatial relationships, including any hydrogen bonding and electrostatic interactions. As described above, peptides are obvious choices for the production of such morphogen analogs because they can provide all of the necessary functional groups and can assume appropriate three-dimensional structures. Several examples of peptide analogs of the finger regions are described herein, below. The peptides are cyclized to maintain hydrogen bonds and create a structure that mimics that of the template. These peptides are synthesized from a linear primary sequence of amino acids in finger 2. An alternative peptide can be created, for example, which combines portions of finger 1 and finger 2, constructed to mimic the structure of the tips of fingers 1 and 2 together as they occur in the folded OP-1 monomer. Biologically active peptides such as F2, F3 or others, then can be used as is or, more preferably, become lead compounds for iterative modification to create a compound that is more stable or more active in vivo. For example, the peptide backbone can be reduced or replaced to reduce hydrolysis in vivo. Alternatively, structural modifications can be introduced to the backbone or by amino acid substitutions that more accurately mimic the protein's structure when bound to the receptor. These second generation structures then can be tested for enhanced binding. In addition, iterative amino acid replacements with alanines, (“alanine scan”) can be used to determine the minimum residue contacts required for binding.

Once these minimum functional groups are known, a fully synthetic molecule can be created which mimics the charge or electrostatic distribution of the minimum required functional groups, and provides the appropriate bulk and structure to functionally mimic a second-generation molecule having the desired binding affinity.

In other embodiments, the subject analogs are peptidomimetics of BMP, such as OP-1, analog peptides. Peptidomimetics are compounds based on, or derived from, peptides and proteins. The analog peptidomimetics of the present invention typically can be obtained by structural modification of a known analog peptide sequence using unnatural amino acids, conformational restraints, isosteric replacement, and the like. The subject peptidomimetics constitute the continum of structural space between peptides and non-peptide synthetic structures; analog peptidomimetics may be useful, therefore, in delineating pharmacophores and in helping to translate peptides into nonpeptide compounds with the activity of the parent analog peptides.

Moreover, as is apparent from the present disclosure, mimetopes of the subject analog peptides can be provided. Such peptidomimetics can have such attributes as being non-hydrolyzable (e.g., increased stability against proteases or other physiological conditions which degrade the corresponding peptide), increased specificity and/or potency, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988, p123), C-7 mimics (Huffman et al. in Peptides: Chemistry and Biology, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modifed (Roark et al. in Peptides: Chemistry and Biology, G. R. Marshall ed., ESCOM Publisher: Leiden, Netherlands, 1988, p134). Also, see generally, Session III: Analytic and synthetic methods, in Peptides: Chemistry and Biology, G. R. Marshall ed., ESCOM Publisher: Leiden, Netherlands, 1988)

In addition to a variety of sidechain replacements that can be carried out to generate the subject OP-1 analog peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

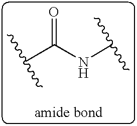

Examples of Surrogates

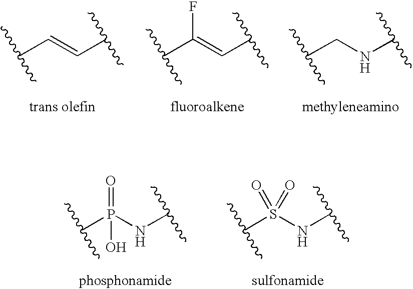

Additionally, peptidomimetics based on more substantial modifications of the backbone of the OP-1 analog peptide can be used. Peptidomimetics which fall in this category include (i) retro-inverso analogs, and (ii) N-alkyl glycine analogs (so-called peptoids).

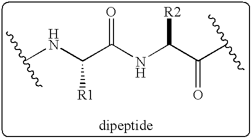

Examples of analogs

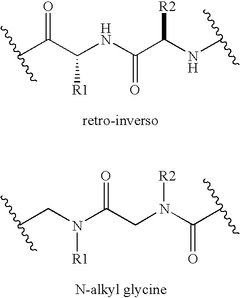

Furthermore, the methods of combinatorial chemistry are being brought to bear, e.g., by G. L. Verdine at Harvard University, on the development of new peptidomimetics. For example, one embodiment of a so-called “peptide morphing” strategy focuses on the random generation of a library of peptide analogs that comprise a wide range of peptide bond substitutes.

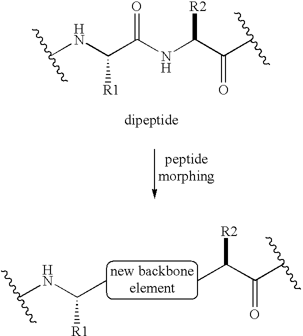

In an exemplary embodiment, the peptidomimetic can be derived as a retro-inverso analog of the peptide

Retro-inverso analogs can be made according to the methods known in the art, such as that described by the Sisto et al. U.S. Pat. No. 4,522,752. As a general guide, sites which are most susceptible to proteolysis are typically altered, with less susceptible amide linkages being optional for mimetic switching. The final product, or intermediates thereof, can be purified by HPLC.

In another illustrative embodiment, the peptidomimetic can be derived as a retro-enantio analog of a particular analog peptide sequence. Retro-enantio analogs such as this can be synthesized commercially available D-amino acids (or analogs thereof) and standard solid- or solution-phase peptide-synthesis techniques.

In still another illustrative embodiment, trans-olefin derivatives can be made for any of the subject polypeptides. A trans-olefin analog of a BMP analog peptide can be synthesized according to the method of Y. K. Shue et al. (1987) Tetrahedron Letters 28:3225 and also according to other methods known in the art. It will be appreciated that variations in the cited procedure, or other procedures available, may be necessary according to the nature of the reagent used.

It is further possible couple the pseudodipeptides synthesized by the above method to other pseudodipeptides, to make peptide analogs with several olefinic functionalities in place of amide functionalities. For example, pseudodipeptides corresponding to certain dipeptide sequences could be made and then coupled together by standard techniques to yield an analog of the OP-1 analog peptide which has alternating olefinic bonds between residues.

Still another class of peptidomimetic derivatives include phosphonate derivatives. The synthesis of such phosphonate derivatives can be adapted from known synthesis schemes. See, for example, Loots et al. in Peptides: Chemistry and Biology, (Escom Science Publishers, Leiden, 1988, p. 118); Petrillo et al. in Peptides: Structure and Function (Proceedings of the 9th American Peptide Symposium, Pierce Chemical Co. Rockland, Ill., 1985).

Many other peptidomimetic structures are known in the art and can be readily adapted for use in the subject OP-1 analog peptidomimetics. To illustrate, the analog peptidomimetic may incorporate the 1-azabicyclo[4.3.0]nonane surrogate (see Kim et al. (1997) J. Org. Chem. 62:2847), or an N-acyl piperazic acid (see Xi et al. (1998) J. Am. Chem. Soc. 120:80), or a 2-substituted piperazine moiety as a constrained amino acid analogue (see Williams et al. (1996) J. Med. Chem. 39:1345-1348). In still other embodiments, certain amino acid residues can be replaced with aryl and biaryl moieties, e.g., monocyclic or bicyclic aromatic or heteroaromatic nucleus, or a biaromatic, aromatic-heteroaromatic, or biheteroaromatic nucleus.

The subject analog peptidomimetics can be optimized by, e.g., combinatorial synthesis techniques combined with such high throughput screening as described above using affinity maturation of the library on bladder tumor cells and selection of specific binding moieties by counterscreening using normal bladder cells.

Moreover, other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds for compounds capable of binding to the BMP binding domain or inhibiting the interaction between the BMP binding domain and the natural ligand. A mimetope can also be obtained, for example, from libraries of natural and synthetic compounds, in particular, chemical or combinatorial libraries (i.e., libraries of compounds that differ in sequence or size but that have the same building blocks). A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modelling. The predicted mimetope structures can then be produced by, for example, chemical synthesis, recombinant DNA technology, or by isolating a mimetope from a natural source (e.g., plants, animals, bacteria and fungi).

(iii) Chimeric BMP Analog Peptides and Peptidomimetics

In one aspect, the invention provides chimeric proteins that include one or more analog peptides fused to one or more additional protein domains. In one embodiment, the chimeric protein includes one analog peptide. In other embodiments, the chimeric activator comprises two or more analog peptides, three or more, five or more, or ten or more analog peptides that are covalently linked. When referring to a polypeptide comprising an analog peptide it is meant that the polypeptide comprises the amino acid sequence of a analog peptide covalently linked to other amino acids or peptides to form one polypeptide. The order of the analog peptide(s) relative to each other and relative to the other domains of the fusion protein can be as desired.

Techniques for making the subject fusion proteins are adapted from well-known procedures. Essentially, the joining of various DNA fragments coding for different polypeptide sequences is performed in accordance with conventional techniques, employing blunt-ended or stagger-ended termini for ligation, restriction enzyme digestion to provide for appropriate termini, filling in of cohesive ends as appropriate, alkaline phosphatase treatment to avoid undesirable joining, and enzymatic ligation. Alternatively, the fusion gene can be synthesized by conventional techniques including automated DNA synthesizers. In another method, PCR amplification of gene fragments can be carried out using anchor primers which give rise to complementary overhangs between two consecutive gene fragments. Amplification products can subsequently be annealed to generate a chimeric gene sequence (see, for example, Current Protocols in Molecular Biology, Eds. Ausubel et al. John Wiley & Sons: 1992).

In certain embodiments, polyanionic or polycatonic binding agents such as oligonucleotides, heparin, lentinan and similar polysaccharide chains, polyamino peptides such as polyaspartate, polyglutamate, polylysine and polyarginine, or other binding agents that maintain a number of either negative or positive charges over their structure at physiological pH's, can be used to specifically bind the subject analog peptides or peptidomimetics. In certain preferred embodiments, a polyanionic component is used, such as heparin, pentosan polysulfate, polyaspartate, polyglutamate, chondroitin sulfate, heparan sulfate, citrate, nephrocalcin, or osteopontin, to name but a few.

Additional domains may be included in the subject fusion proteins of this invention. For example, the fusion proteins may include domains that facilitate their purification, e.g., “histidine tags” or a glutathione-S-transferase domain. They may include “epitope tags” encoding peptides recognized by known monoclonal antibodies for the detection of proteins within cells or the capture of proteins by antibodies in vitro.

It may be necessary in some instances to introduce an unstructured polypeptide linker region between an analog peptide and other portions of the chimeric protein. The linker can facilitate enhanced flexibility of the fusion protein. The linker can also reduce steric hindrance between any two fragments of the fusion protein. The linker can also facilitate the appropriate folding of each fragment to occur. The linker can be of natural origin, such as a sequence determined to exist in random coil between two domains of a protein. An exemplary linker sequence is the linker found between the C-terminal and N-terminal domains of the RNA polymerase a subunit. Other examples of naturally occurring linkers include linkers found in the lcI and LexA proteins. Alternatively, the linker can be of synthetic origin. For instance, the sequence (Gly₄Ser)₃ can be used as a synthetic unstructured linker. Linkers of this type are described in Huston et al. (1988) PNAS 85:4879; and U.S. Pat. No. 5,091,513.

In some embodiments it is preferable that the design of a linker involve an arrangement of domains which requires the linker to span a relatively short distance, preferably less than about 10 Å. However, in certain embodiments, depending, e.g., upon the selected domains and the configuration, the linker may span a distance of up to about 50 Å.

Within the linker, the amino acid sequence may be varied based on the preferred characteristics of the linker as determined empirically or as revealed by modeling. For instance, in addition to a desired length, modeling studies may show that side groups of certain amino acids may interfere with the biological activity of the fusion protein. Considerations in choosing a linker include flexibility of the linker, charge of the linker, and presence of some amino acids of the linker in the naturally occurring subunits. The linker can also be designed such that residues in the linker contact DNA, thereby influencing binding affinity or specificity, or to interact with other proteins. For example, a linker may contain an amino acid sequence that is recognized by a protease so that the activity of the chimeric protein could be regulated by cleavage. In some cases, particularly when it is necessary to span a longer distance between subunits or when the domains must be held in a particular configuration, the linker may optionally contain an additional folded domain.

VL. Production of Morphogen Analogs.

As mentioned above, the morphogen analogs of the invention may comprise modified hOP-1 dimeric proteins or small molecules, for example, peptides or small organic molecules. It is contemplated that any appropriate methods can be used for producing a preselected morphogen analog. For example, such methods may include, but are not limited to, methods of biological production from suitable host cells or synthetic production using synthetic organic chemistries.

For example, modified hOP-1 dimeric proteins or hOP-based peptides may be produced using conventional recombinant DNA technologies, well known and thoroughly documented in the art. Under these circumstances, the proteins or peptides may be produced by the preparation of nucleic acid sequences encoding the respective protein or peptide sequences, after which, the resulting nucleic acid can be expressed in an appropriate host cell. By way of example, the proteins and peptides may be manufactured by the assembly of synthetic nucleotide sequences and/or joining DNA restriction fragments to produce a synthetic DNA molecule. The DNA molecules then are ligated into an expression vehicle, for example an expression plasmid, and transfected into an appropriate host cell, for example E. coli. The protein encoded by the DNA molecule then is expressed, purified, folded if necessary, tested in vitro for binding activity with a BMP/OP receptor, and subsequently tested to assess whether the morphogen analog induces or stimulates hOP-1-like biological activity.

The processes for manipulating, amplifying, and recombining DNA which encode amino acid sequences of interest generally are well known in the art, and therefore, are not described in detail herein. Methods of identifying and isolating genes encoding hOP-1 and its cognate receptors also are well understood, and are described in the patent and other literature.

Briefly, the construction of DNAs encoding the biosynthetic constructs disclosed herein is performed using known techniques involving the use of various restriction enzymes which make sequence specific cuts in DNA to produce blunt ends or cohesive ends, DNA ligases, techniques enabling enzymatic addition of sticky ends to blunt-ended DNA, construction of synthetic DNAs by assembly of short or medium length oligonucleotides, cDNA synthesis techniques, polymerase chain reaction (PCR) techniques for amplifying appropriate nucleic acid sequences from libraries, and synthetic probes for isolating OP-1 genes or genes encoding other members of the TGF-β superfamily as well as their cognate receptors. Various promoter sequences from bacteria, mammals, or insects to name a few, and other regulatory DNA sequences used in achieving expression, and various types of host cells are also known and available. Conventional transfection techniques, and equally conventional techniques for cloning and subcloning DNA are useful in the practice of this invention and known to those skilled in the art. Various types of vectors may be used such as plasmids and viruses including animal viruses and bacteriophages. The vectors may exploit various marker genes that impart to a successfully transfected cell a detectable phenotypic property that can be used to identify which of a family of clones has successfully incorporated the recombinant DNA of the vector.

One method for obtaining DNA encoding the biosynthetic constructs disclosed herein is by assembly of synthetic oligonucleotides produced in a conventional, automated, oligonucleotide synthesizer followed by ligation with appropriate ligases. For example, overlapping, complementary DNA fragments may be synthesized using phosphoramidite chemistry, with end segments left unphosphorylated to prevent polymerization during ligation. One end of the synthetic DNA is left with a “sticky end” corresponding to the site of action of a particular restriction endonuclease, and the other end is left with an end corresponding to the site of action of another restriction endonuclease. The complementary DNA fragments are ligated together to produce a synthetic DNA construct.

After the appropriate DNA molecule has been synthesized, it may be integrated into an expression vector and transfected into an appropriate host cell for protein expression. Useful prokaryotic host cells include, but are not limited to, E. coli and B. subtilis. Useful eukaryotic host cells include, but are not limited to, yeast cells, insect cells, myeloma cells, fibroblast 3T3 cells, monkey kidney or COS cells, chinese hamster ovary (CHO) cells, mink-lung epithelial cells, human foreskin fibroblast cells, human glioblastoma cells, and teratocarcinoma cells. Alternatively, the genes may be expressed in a cell-free system such as the rabbit reticulocyte lysate system.

The vector additionally may include various sequences to promote correct expression of the recombinant protein, including transcriptional promoter and termination sequences, enhancer sequences, preferred ribosome binding site sequences, preferred mRNA leader sequences, preferred protein processing sequences, preferred signal sequences for protein secretion, and the like. The DNA sequence encoding the gene of interest also may be manipulated to remove potentially inhibiting sequences or to minimize unwanted secondary structure formation. The morphogenic protein analogs proteins also may be expressed as fusion proteins. After being translated, the protein may be purified from the cells themselves or recovered from the culture medium and then cleaved at a specific protease site if so desired.

For example, if the gene is to be expressed in E. coli, it is cloned into an appropriate expression vector. This can be accomplished by positioning the engineered gene downstream of a promoter sequence such as Trp or Tac, and/or a gene coding for a leader peptide such as fragment B of protein A (FB). During expression, the resulting fusion proteins accumulate in refractile bodies in the cytoplasm of the cells, and may be harvested after disruption of the cells by French press or sonication. The isolated refractile bodies then are solubilized, and the expressed proteins folded and the leader sequence cleaved, if necessary, by methods already established with many other recombinant proteins.

Expression of the engineered genes in eukaryotic cells requires cells and cell lines that are easy to transfect, are capable of stably maintaining foreign DNA with an unrearranged sequence, and which have the necessary cellular components for efficient transcription, translation, post-translation modification, and secretion of the protein. In addition, a suitable vector carrying the gene of interest also is necessary. DNA vector design for transfection into mammalian cells should include appropriate sequences to promote expression of the gene of interest as described herein, including appropriate transcription initiation, termination, and enhancer sequences, as well as sequences that enhance translation efficiency, such as the Kozak consensus sequence. Preferred DNA vectors also include a marker gene and means for amplifying the copy number of the gene of interest. A detailed review of the state of the art of the production of foreign proteins in mammalian cells, including useful cells, protein expression-promoting sequences, marker genes, and gene amplification methods, is disclosed in Bendig (1988) Genetic Engineering 7:91-127.

The best characterized transcription promoters useful for expressing a foreign gene in a particular mammalian cell are the SV40 early promoter, the adenovirus promoter (AdMLP), the mouse metallothionein-I promoter (mMT-I), the Rous sarcoma virus (RSV) long terminal repeat (LTR), the mouse mammary tumor virus long terminal repeat (MMTV-LTR), and the human cytomegalovirus major intermediate-early promoter (hCMV). The DNA sequences for all of these promoters are known in the art and are available commercially.

The use of a selectable DHFR gene in a dhfr⁻ cell line is a well characterized method useful in the amplification of genes in mammalian cell systems. Briefly, the DHFR gene is provided on the vector carrying the gene of interest, and addition of increasing concentrations of the cytotoxic drug methotrexate, which is metabolized by DHFR, leads to amplification of the DHFR gene copy number, as well as that of the associated gene of interest. DHFR as a selectable, amplifiable marker gene in transfected Chinese hamster ovary cell lines (CHO cells) is particularly well characterized in the art. Other useful amplifiable marker genes include the adenosine deaminase (ADA) and glutamine synthetase (GS) genes.

The choice of cells/cell lines is also important and depends on the needs of the experimenter. COS cells provide high levels of transient gene expression, providing a useful means for rapidly screening the biosynthetic constructs of the invention. COS cells typically are transfected with a simian virus 40 (SV40) vector carrying the gene of interest. The transfected COS cells eventually die, thus preventing the long term production of the desired protein product but provide a useful technique for testing preliminary analogs for binding activity.

The various cells, cell lines and DNA sequences that can be used for mammalian cell expression of the single-chain constructs of the invention are well characterized in the art and are readily available. Other promoters, selectable markers, gene amplification methods and cells also may be used to express the proteins of this invention. Particular details of the transfection, expression, and purification of recombinant proteins are well documented in the art and are understood by those having ordinary skill in the art. Further details on the various technical aspects of each of the steps used in recombinant production of foreign genes in mammalian cell expression systems can be found in a number of texts and laboratory manuals in the art, such as, for example, Ausubel et al., ed., Current Protocols in Molecular Biology, John Wiley & Sons, New York, (1989).

In addition, vectors suitable for mammalian cell expression can be used in gene therapy treatment protocols, whereby the peptide is produced in the cells of the patient being treated. In yet other embodiments, the subject expression constructs are derived by insertion of the subject gene into viral vectors including recombinant retroviruses, adenovirus, adeno-associated virus, and herpes simplex virus-1, or recombinant bacterial or eukaryotic plasmids. As described in greater detail below, such embodiments of the subject expression constructs are specifically contemplated for use in various in vivo and ex vivo gene therapy protocols.

Retrovirus vectors and adeno-associated virus vectors are generally understood to be the recombinant gene delivery system of choice for the transfer of exogenous genes in vivo, particularly into humans. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes (for a review see Miller, A. D. (1990) Blood 76:271). Thus, recombinant retrovirus can be constructed in which part of the retroviral coding sequence (gag, pol, env) has been replaced by nucleic acid encoding a fusion protein of the present invention, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al., (eds.) Greene Publishing Associates, (1989), Sections 9.10-9.14 and other standard laboratory manuals. Examples of suitable retroviruses include pLJ, pZIP, pWE and pEM which are well known to those skilled in the art. Retroviruses have been used to introduce a variety of genes into many different cell types, including neural cells, epithelial cells, endothelial cells, lymphocytes, myoblasts, hepatocytes, bone marrow cells, in vitro and/or in vivo (see for example Eglitis et al., (1985) Science 230:1395-1398; Danos and Mulligan, (1988) PNAS USA 85:6460-6464; Wilson et al., (1988) PNAS USA 85:3014-3018; Armentano et al., (1990) PNAS USA 87:6141-6145; Huber et al., (1991) PNAS USA 88:8039-8043; Ferry et al., (1991) PNAS USA 88:8377-8381; Chowdhury et al., (1991) Science 254:1802-1805; van Beusechem et al., (1992) PNAS USA 89:7640-7644; Kay et al., (1992) Human Gene Therapy 3:641-647; Dai et al., (1992) PNAS USA 89:10892-10895; Hwu et al., (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Furthermore, it has been shown that it is possible to limit the infection spectrum of retroviruses and consequently of retroviral-based vectors, by modifying the viral packaging proteins on the surface of the viral particle (see, for example PCT publications WO93/25234, WO94/06920, and WO94/11524). For instance, strategies for the modification of the infection spectrum of retroviral vectors include: coupling antibodies specific for cell surface antigens to the viral env protein (Roux et al., (1989) PNAS USA 86:9079-9083; Julan et al., (1992) J. Gen Virol 73:3251-3255; and Goud et al., (1983) Virology 163:251-254); or coupling cell surface ligands to the viral env proteins (Neda et al., (1991) J. Biol. Chem. 266:14143-14146). Coupling can be in the form of the chemical cross-linking with a protein or other variety (e.g., lactose to convert the env protein to an asialoglycoprotein), as well as by generating fusion proteins (e.g., single-chain antibody/env fusion proteins). This technique, while useful to limit or otherwise direct the infection to certain tissue types, and can also be used to convert an ecotropic vector in to an amphotropic vector.

Another viral gene delivery system useful in the present invention utilizes adenovirus-derived vectors. The genome of an adenovirus can be manipulated such that it encodes a gene product of interest, but is inactivate in terms of its ability to replicate in a normal lytic viral life cycle (see, for example, Berkner et al., (1988) BioTechniques 6:616; Rosenfeld et al., (1991) Science 252:431-434; and Rosenfeld et al., (1992) Cell 68:143-155). Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses can be advantageous in certain circumstances in that they are not capable of infecting nondividing cells and can be used to infect a wide variety of cell types, including airway epithelium (Rosenfeld et al., (1992) cited supra), endothelial cells (Lemarchand et al., (1992) PNAS USA 89:6482-6486), hepatocytes (Herz and Gerard, (1993) PNAS USA 90:2812-2816) and muscle cells (Quantin et al., (1992) PNAS USA 89:2581-2584). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham (1986) J. Virol. 57:267). Most replication-defective adenoviral vectors currently in use and therefore favored by the present invention are deleted for all or parts of the viral E1 and E3 genes but retain as much as 80% of the adenoviral genetic material (see, e.g., Jones et al., (1979) Cell 16:683; Berkner et al., supra; and Graham et al., in Methods in Molecular Biology, E. J. Murray, Ed. (Humana, Clifton, N.J., 1991) vol. 7. pp. 109-127). Expression of the inserted chimeric gene can be under control of, for example, the E1A promoter, the major late promoter (MLP) and associated leader sequences, the viral E3 promoter, or exogenously added promoter sequences.

Yet another viral vector system useful for delivery of the subject chimeric genes is the adeno-associated virus (AAV). Adeno-associated virus is a naturally occurring defective virus that requires another virus, such as an adenovirus or a herpes virus, as a helper virus for efficient replication and a productive life cycle. (For a review, see Muzyczka et al., Curr. Topics in Micro. and Immunol. (1992) 158:97-129). It is also one of the few viruses that may integrate its DNA into non-dividing cells, and exhibits a high frequency of stable integration (see for example Flotte et al., (1992) Am. J. Respir. Cell. Mol. Biol. 7:349-356; Samulski et al., (1989) J. Virol. 63:3822-3828; and McLaughlin et al., (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al., (1985) Mol. Cell. Biol. 5:3251-3260 can be used to introduce DNA into cells. A variety of nucleic acids have been introduced into different cell types using AAV vectors (see for example Hermonat et al., (1984) PNAS USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flotte et al., (1993) J. Biol. Chem. 268:3781-3790).

Other viral vector systems that may have application in gene therapy have been derived from herpes virus, vaccinia virus, and several RNA viruses. In particular, herpes virus vectors may provide a unique strategy for persistence of the recombinant gene in cells of the central nervous system and ocular tissue (Pepose et al., (1994) Invest Ophthalmol Vis Sci 35:2662-2666)

In addition to viral transfer methods, such as those illustrated above, non-viral methods can also be employed to cause expression of a protein in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the gene by the targeted cell. Exemplary gene delivery systems of this type include liposomal derived systems, poly lysine conjugates, and artificial viral envelopes.

In a representative embodiment, a gene encoding a polypeptide can be entrapped in liposomes bearing positive charges on their surface (e.g., lipofectins) and (optionally) which are tagged with antibodies against cell surface antigens of the target tissue (Mizuno et al., (1992) No Shinkei Geka 20:547-551; PCT publication WO91/06309; Japanese patent application 1047381; and European patent publication EP-A-43075). For example, lipofection of neuroglioma cells can be carried out using liposomes tagged with monoclonal antibodies against glioma-associated antigen (Mizuno et al., (1992) Neurol. Med. Chir. 32:873-876).

In yet another illustrative embodiment, the gene delivery system comprises an antibody or cell surface ligand which is cross-linked with a gene binding agent such as poly lysine (see, for example, PCT publications WO93104701, WO92/22635, WO92/20316, WO92/19749, and WO92/06180). For example, any of the subject gene constructs can be used to transfect specific cells in vivo using a soluble polynucleotide carrier comprising an antibody conjugated to a polycation, e.g., poly lysine (see U.S. Pat. No. 5,166,320). It will also be appreciated that effective delivery of the subject nucleic acid constructs via-mediated endocytosis can be improved using agents which enhance escape of the gene from the endosomal structures. For instance, whole adenovirus or fusogenic peptides of the influenza HA gene product can be used as part of the delivery system to induce efficient disruption of DNA-containing endosomes (Mulligan et al., (1993) Science 260-926; Wagner et al., (1992) PNAS USA 89:7934; and Christiano et al., (1993) PNAS USA 90:2122).

In clinical settings, the gene delivery systems can be introduced into a patient by any of a number of methods, each of which is familiar in the art. For instance, a pharmaceutical preparation of the gene delivery system can be introduced systemically, e.g., by intravenous injection, and specific transduction of the construct in the target cells occurs predominantly from specificity of transfection provided by the gene delivery vehicle, cell-type or tissue-type expression due to the transcriptional regulatory sequences controlling expression of the gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g., Chen et al., (1994) PNAS USA 91: 3054-3057).

Alternatively, morphogen analogs which are small peptides, usually up to 50 amino acids in length, may be synthesized using standard solid-phase peptide synthesis procedures, for example, procedures similar to those described in Merrifield (1963) J. Am. Chem. Soc., 85:2149. For example, during synthesis, N-α-protected amino acids having protected side chains are added stepwise to a growing polypeptide chain linked by its C-terminal end to an insoluble polymeric support, e.g., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. Commonly used N-α-protecting groups include Boc, which is acid labile, and Fmoc, which is base labile.

Briefly, the C-terminal N-α-protected amino acid is first attached to the polystyrene beads. Then, the N-α-protecting group is removed. The deprotected α-amino group is coupled to the activated α-carboxylate group of the next N-α-protected amino acid. The process is repeated until the desired peptide is synthesized. The resulting peptides are cleaved from the insoluble polymer support and the amino acid side chains deprotected. Longer peptides, for example greater than about 50 amino acids in length, typically are derived by condensation of protected peptide fragments. Details of appropriate chemistries, resins, protecting groups, protected amino acids and reagents are well known in the art and so are not discussed in detail herein. See for example, Atherton et al. (1963) Solid Phase Peptide Synthesis: A Practical Approach (IRL Press,), and Bodanszky (1993) Peptide Chemistry, A Practical Textbook, 2nd Ed., Springer-Verlag, and Fields et al. (1990) Int. J. Peptide Protein Res. 35:161-214, the disclosures of which are incorporated herein by reference.

Purification of the resulting peptide is accomplished using conventional procedures, such as preparative HPLC, e.g., gel permeation, partition and/or ion exchange chromatography. The choice of appropriate matrices and buffers are well known in the art and so are not described in detail herein.

With regard to the production of non-peptide small organic molecules that induce BMP/OP-1 like biological activities, these molecules can be synthesized using standard organic chemistries well known and thoroughly documented in the patent and other literatures.

VI. Screening for Binding and Biological Activity

As a first step in determining whether a morphogen analog induces an OP-1 like biological activity, the skilled artisan can use a standard ligand-receptor assay to determine whether the morphogen analog binds preferentially to a BMP receptor, such as an OP-1 receptor. For standard receptor-ligand assays, the artisan is referred to, for example, Legerski et al. (1992) Biochem. Biophys. Res. Comm. 183: 672-679; Frakar et al. (1978) Biochem. Biophys. Res. Comm. 80:849-857; Chio et al. (1990) Nature 343: 266-269; Dahlman et al. (1988) Biochem 27: 1813-1817; Strader et al. (1989) J. Biol. Chem. 264: 13572-13578; and D'Dowd et al. (1988) J. Biol. Chem. 263: 15985-15992.

In a typical ligand/receptor-binding assay useful in the practice of this invention, purified OP-1 having a known, quantifiable affinity for a pre-selected OP-1 receptor (see, for example, Ten Dijke et al. (1994) Science 264:101-103, the disclosure of which is incorporated herein by reference) is labeled with a detectable moiety, for example, a radiolabel, a chromogenic label, or a fluorogenic label. Aliquots of purified receptor, receptor-binding domain fragments, or cells expressing the receptor of interest on their surface are incubated with labeled OP-1 in the presence of various concentrations of the unlabeled morphogen analog. The relative binding affinity of the morphogen analog may be measured by quantitating the ability of the candidate (unlabeled morphogen analog) to inhibit the binding of labeled OP-1 with the receptor. In performing the assay, fixed concentrations of the receptor and the OP-1 are incubated in the presence and absence of unlabeled morphogen analog. Sensitivity may be increased by pre-incubating the receptor with the candidate morphogen analog before adding labeled OP-1. After the labeled competitor has been added, sufficient time is allowed for adequate competitor binding, and then free and bound labeled OP-1 are separated from one another, and one or the other measured.

Labels useful in the practice of the screening procedures include radioactive labels (e.g., ¹²⁵I, ¹³¹I, ¹¹¹In or ⁷⁷Br), chromogenic labels, spectroscopic labels (such as those disclosed in Haughland (1994) “Handbook of Fluorescent and Research Chemicals 5 ed.” by Molecular Probes, Inc., Eugene, Oreg.), or conjugated enzymes having high turnover rates, for example, horseradish peroxidase, alkaline phosphatase, or β-galactosidase, used in combination with chemiluminescent or fluorogenic substrates.

The biological activity, namely the agonist or antagonist properties of the resulting morphogen analogs subsequently may be characterized using any conventional in vivo and in vitro assays that have been developed to measure the biological activity of a BMP, such as OP-1. A variety of specific assays believed to be useful in the practice of the invention are set forth in detail in Example 1, hereinbelow.

Furthermore, it is appreciated that many of the standard OP-1 assays may be automated thereby facilitating the screening of a large number of morphogen analogs at the same time. Such automation procedures are within the level of skill in the art of drug screening and, therefore, are not discussed herein.

Following the identification of useful morphogen analogs, the morphogenic analogs may be produced in commercially useful quantities (e.g., without limitation, gram and kilogram quantities), for example, by producing cell lines that express the morphogen analogs of interest or by producing synthetic peptides defining the appropriate amino acid sequence. It is appreciated, however, that conventional methodologies for producing the appropriate cell lines and for producing synthetic peptides are well known and thoroughly documented in the art, and so are not discussed in detail herein.

An additional approach employs nuclear magnetic resonance (NMR)-based screening to rapidly identify potentially useful morphogen analogs based on their ability to bind to a protein. Recent advances in this field have reduced the noise to signal ratios in this assay, and facilitated the high-throughput screening of libraries of compounds (see, for example, Hajduk et al. (1999) Journal of Medicinal Chemistry 42: 2315). In fact, Hajduk et al. demonstrate that this technique is sufficiently sensitive to distinguish a single “positive” receptor-compound interaction among a pool of 100 non-interacting compounds. Given the pace with which this assay can be performed, an estimated 10,000 compounds can be screened in one day.

In the context of the present invention, the ecto-domain of a type-I or type-II BMP receptor is labeled, and NMR-spectra acquired using standard methods as exemplified by Hajduk et al. The labeled BMP receptor is then contacted with a pool of compounds, and the NMR-spectra in the presence of the pool of compounds is acquired. Comparison of the NMR-spectra in the presence and absence of the pool of compounds is an indicator of an interaction between a compound in the pool and the BMP-receptor, wherein an interaction results in a shift of the chemical spectra. In one embodiment, the BMP-receptor is labeled with ¹⁵N, and screening comprises detecting a change in the ¹⁵N/¹H amide chemical shift. Accordingly, such as NMR-based approach can be used to screen potential BMP analogs. This method allows for high-throughput screening of potential BMP analogs, and has the additional advantage of being amenable to the screening of a range of compounds including both peptides and small molecules.

VIII. Formulation and Bioactivity

Morphogen analogs, including OP-1 analogs, can be formulated for administration to a mammal, preferably a human in need thereof as part of a pharmaceutical composition. The composition can be administered by any suitable means, e.g., parenterally, orally or locally. Where the morphogen analog is to be administered locally, as by injection, to a desired tissue site, or systemically, such as by intravenous, subcutaneous, intramuscular, intraorbital, ophthalmic, intraventricular, intracranial, intracapsular, intraspinal, intracisternal, intraperitoneal, buccal, rectal, vaginal, intranasal or aerosol administration, the composition preferably comprises an aqueous solution. The solution preferably is physiologically acceptable, such that administration thereof to a mammal does not adversely affect the mammal's normal electrolyte and fluid volume balance. The aqueous solution thus can comprise, e.g., normal physiologic saline (0.9% NaCl, 0.1 5M), pH 7-7.4.

Useful solutions for oral or parenteral systemic administration can be prepared by any of the methods well known in the pharmaceutical arts, described, for example, in “Remington's Pharmaceutical Sciences” (Gennaro, A., ed., Mack Pub., 1990, the disclosure of which is incorporated herein by reference). Formulations can include, for example, polyalkylene glycols such as polyethylene glycol, oils of vegetable origin, hydrogenated naphthalenes, and the like. Formulations for direct administration, in particular, can include glycerol and other compositions of high viscosity. Biocompatible, preferably bioresorbable polymers, including, for example, hyaluronic acid, collagen, tricalcium phosphate, polybutyrate, polylactide, polyglycolide and lactide/glycolide copolymers, may be useful excipients to control the release of the morphogen analog in vivo.

Other potentially useful parenteral delivery systems for the present analogs can include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate or deoxycholate, or oily solutions for administration in the form of nasal drops or as a gel to be applied intranasally.

Alternatively, the morphogen analogs, including OP-1 analogs, identified as described herein may be administered orally. For example, liquid formulations of morphogen analogs can be prepared according to standard practices such as those described in “Remington's Pharmaceutical Sciences” (supra). Such liquid formulations can then be added to a beverage or another food supplement for administration. Oral administration can also be achieved using aerosols of these liquid formulations. Alternatively, solid formulations prepared using art-recognized emulsifiers can be fabricated into tablets, capsules or lozenges suitable for oral administration.

Optionally, the analogs can be formulated in compositions comprising means for enhancing uptake of the analog by a desired tissue. For example, tetracycline and diphosphonates (bisphosphonates) are known to bind to bone mineral, particularly at zones of bone remodeling, when they are provided systemically in a mammal. Accordingly, such components can be used to enhance delivery of the present analogs to bone tissue. Alternatively, an antibody or portion thereof that binds specifically to an accessible substance specifically associated with the desired target tissue, such as a cell surface antigen, also can be used. If desired, such specific targeting molecules can be covalently bound to the present analog, e.g., by chemical crosslinking or by using standard genetic engineering techniques to create, for example, an acid labile bond such as an Asp-Pro linkage. Useful targeting molecules can be designed, for example, according to the teachings of U.S. Pat. No. 5,091,513.

It is contemplated also that some of the morphogen analogs may exhibit the highest levels of activity in vivo when combined with carrier matrices, i.e., insoluble polymer matrices. See for example, U.S. Pat. No. 5,266,683 the disclosure of which is incorporated by reference herein. Currently preferred carrier matrices are xenogenic, allogenic or autogenic in nature. It is contemplated, however, that synthetic materials comprising polylactic acid, polyglycolic acid, polybutyric acid, derivatives and copolymers thereof may also be used to generate suitable carrier matrices. Preferred synthetic and naturally derived matrix materials, their preparation, methods for formulating them with the morphogen analogs of the invention, and methods of administration are well known in the art and so are not discussed in detailed herein. See for example, U.S. Pat. No. 5,266,683.

Still further, the present analogs can be administered to the mammal in need thereof either alone or in combination with another substance known to have a beneficial effect on tissue morphogenesis. Examples of such substances (herein, cofactors) include substances that promote tissue repair and regeneration and/or inhibit inflammation. Examples of useful cofactors for stimulating bone tissue growth in osteoporotic individuals, for example, include but are not limited to, vitamin D₃, calcitonin, prostaglandins, parathyroid hormone, dexamethasone, estrogen and IGF-I or IGF-II. Useful cofactors for nerve tissue repair and regeneration can include nerve growth factors. Other useful cofactors include symptom-alleviating cofactors, including antiseptics, antibiotics, antiviral and antifungal agents, analgesics and anesthetics.

Analogs preferably are formulated into pharmaceutical compositions by admixture with pharmaceutically acceptable, nontoxic excipients and carriers. As noted above, such compositions can be prepared for systemic, e.g., parenteral, administration, particularly in the form of liquid solutions or suspensions; for oral administration, particularly in the form of tablets or capsules; or intranasally, particularly in the form of powders, nasal drops or aerosols. Where adhesion to a tissue surface is desired, the composition can comprise a fibrinogen-thrombin dispersant or other bioadhesive such as is disclosed, for example, in PCT US91/09275, the disclosure of which is incorporated herein by reference. The composition then can be painted, sprayed or otherwise applied to the desired tissue surface.

The compositions can be formulated for parenteral or oral administration to humans or other mammals in therapeutically effective amounts, e.g., amounts that provide appropriate concentrations of the morphogen analog to target tissue for a time sufficient to induce the desired effect. Preferably, the present compositions alleviate or mitigate the mammal's need for a morphogen-associated biological response, such as maintenance of tissue-specific function or restoration of tissue-specific phenotype to senescent tissues (e.g., osteopenic bone tissue).

As will be appreciated by those skilled in the art, the concentration of the compounds described in a therapeutic composition will vary depending upon a number of factors, including the dosage of the drug to be administered, the chemical characteristics (e.g., hydrophobicity) of the compounds employed, and the route of administration. The preferred dosage of drug to be administered also is likely to depend on such variables as the type and extent of a disease, tissue loss or defect, the overall health status of the particular patient, the relative biological efficacy of the compound selected, the formulation of the compound, the presence and types of excipients in the formulation, and the route of administration. In general terms, the therapeutic molecules of this invention may be provided to an individual where typical doses range from about 10 ng/kg to about 1 g/kg of body weight per day; with a preferred dose range being from about 0.1 mg/kg to 100 mg/kg of body weight. In a preferred embodiment, the therapeutic molecules may be provided at a dose of 10-100 μg/kg per day. In another preferred embodiment, the therapeutic molecules may be provided at a dose of 30 μg/kg per day. In yet another preferred embodiment of the present invention, the therapeutic molecules may be administered every two days.

### IX. EXAMPLES

### Example 1

Practice of the invention will be more fully understood from the following examples, which are presented herein for illustrative purposes only, and should not be construed as limiting the invention in any way.

The skilled artisan can test an analog for biological activity in any of a number of assays without undue experimentation. Several exemplary assays are discussed below.

A. Mitogenic Effect on Rat and Human Osteoblasts

The following example is a typical assay useful in determining whether a morphogen analog induces proliferation of osteoblasts in vitro. It is contemplated that this and all other examples using osteoblast cultures preferably use rat osteoblast-enriched primary cultures. Although these cultures are heterogeneous in that the individual cells are at different stages of differentiation, the culture is believed to more accurately reflect the metabolism and function of osteoblasts in vivo than osteoblast cultures obtained from established cell lines. Unless otherwise indicated, all chemicals referenced are standard, commercially available reagents, readily available from a number of sources, including Sigma Chemical, Co., St. Louis; Calbiochem, Corp., San Diego and Aldrich Chemical Co., Milwaukee.

Briefly, rat osteoblast-enriched primary cultures are prepared by sequential collagenase digestion of newborn rat calvaria (e.g., from 1-2 day-old animals, Long-Evans strain, Charles River Laboratories, Wilmington, Mass.), following standard procedures, such as are described, for example, in Wong et al. (1975) Proc. Natl. Acad. Sci. USA 72: 3167-3171. Rat osteoblast single cell suspensions then are plated onto a multi-well plate (e.g., a 24 well plate at a concentration of 50,000 osteoblasts per well) in alpha MEM (modified Eagle's medium, Gibco, Inc., N.Y.) containing 10% FBS (fetal bovine serum), L-glutamine and penicillin/streptomycin. The cells are incubated for 24 hours at 37° C., at which time the growth medium is replaced with alpha MEM containing 1% FBS and the cells incubated for an additional 24 hours so that cells are in serum-deprived growth medium at the time of the experiment.

The cultured cells are divided into four groups: (1) wells which receive, for example, 0.1, 1.0, 10.0, 40.0 and 80.0 ng of the morphogen analog (mutein), (2) wells which receive 0.1, 1.0, 10.0 and 40.0 ng of wild type OP-1; (3) wells which receives 0.1, 1.0, 10.0, and 40.0 ng of TGF-β, and (4) the control group, which receive no growth factors. The cells then are incubated for an additional 18 hours after which the wells are pulsed with 2 mCi/well of ³H-thymidine and incubated for six more hours. The excess label then is washed off with a cold solution of 0.15 M NaCl and then 250 ml of 10% tricholoracetic acid is added to each well and the wells incubated at room temperature for 30 minutes. The cells then are washed three times with cold distilled water, and lysed by the addition of 250 ml of 1% sodium dodecyl sulfate (SDS) for a period of 30 minutes at 37° C. The resulting cell lysates are harvested using standard means and the incorporation of ³H-thymidine into cellular DNA determined by liquid scintillation as an indication of mitogenic activity of the cells. In the experiment, it is contemplated that the morphogen analog construct (mutein), like natural OP-1, will stimulate ³H-thymidine incorporation into DNA, and therefore promote osteoblast cell proliferation. In contrast, the effect of the TGF-β is expected to be transient and biphasic. Furthermore, it is contemplated that at higher concentrations, TGF-β will have no significant effect on osteoblast cell proliferation.

The in vitro effect of the morphogen analog on osteoblast proliferation also may be evaluated using human primary osteoblasts (obtained from bone tissue of a normal adult patient and prepared as described above) and on human osteosarcoma-derived cell lines.

B. Progenitor Cell Stimulation

The following example is designed to demonstrate the ability of morphogen analogs to stimulate the proliferation of mesenchymal progenitor cells. Useful naive stem cells include pluripotent stem cells, which may be isolated from bone marrow or umbilical cord blood using conventional methodologies, (see, for example, Faradji et al. (1988) Vox Sang. 55 (3): 133-138 or Broxmeyer et al. (1989) Proc. Natl. Acad. Sci. USA. 86: 3828-3832), as well as naive stem cells obtained from blood. Alternatively, embryonic cells (e.g., from a cultured mesodermal cell line) may be used.

Another method for obtaining progenitor cells and for determining the ability of morphogen analogs to stimulate cell proliferation is to capture progenitor cells from an in vivo source. For example, a biocompatible matrix material able to allow the influx of migratory progenitor cells may be implanted at an in vivo site long enough to allow the influx of migratory progenitor cells. For example, a bone-derived, guanidine-extracted matrix, formulated as disclosed for example in Sampath et al. (1983) Proc. Natl. Acad. Sci. USA 80: 6591-6595, or U.S. Pat. No. 4,975,526, may be implanted into a rat at a subcutaneous site, essentially following the method of Sampath et al. After three days, the implant is removed, and the progenitor cells associated with the matrix dispersed and cultured.

Progenitor cells, however obtained, then are incubated in vitro with the candidate morphogen analog under standard cell culture conditions, such as those described hereinbelow. In the absence of external stimuli, the progenitor cells do not, or only minimally, proliferate on their own in culture. However, progenitor cells cultured in the presence of a biologically active morphogen analog, like OP-1, will proliferate. Cell growth can be determined visually or spectrophotometrically using standard methods well known in the art.

C. Morphogen-Induced Cell Differentiation

A variety of assays also can be used to determine morphogen analog-induced cellular differentiation. (1) Embryonic Mesenchyme Differentiation

As with natural OP-1, it is contemplated that the morphogen analog (mutein) can induce cell differentiation. The ability of morphogen analogs to induce cell differentiation can be demonstrated by culturing early mesenchymal cells in the presence of morphogen analog and then studying the histology of the cultured cells by staining with toluidine blue using standard cell culturing and cell staining methodologies well described in the art. For example, it is known that rat mesenchymal cells destined to become mandibular bone, when separated from the overlying epithelial cells at stage 11 and cultured in vitro under standard tissue culture conditions, e.g., in a chemically defined, serum-free medium, containing for example, 67% DMEM (Dulbecco's modified Eagle's medium), 22% F-12 medium, 10 mM Hepes pH 7, 2 mM glutamine, 50 mg/ml transferrin, 25 mg/ml insulin, trace elements, 2 mg/ml bovine serum albumin coupled to oleic acid, with HAT (0.1 mM hypoxanthine, 10 mM aminopterin, 12 mM thymidine, will not continue to differentiate. However, if these same cells are left in contact with the overlying endoderm for an additional day, at which time they become stage 12 cells, they will continue to differentiate on their own in vitro to form chondrocytes. Further differentiation into osteoblasts and, ultimately, mandibular bone, requires an appropriate local environment, e.g., a vascularized environment.

It is anticipated that, as with natural OP-1, stage 11 mesenchymal cells, cultured in vitro in the presence of morphogen analog (mutein), e.g., 10-100 ng/ml, will continue to differentiate in vitro to form chondrocytes just as they continue to differentiate in vitro if they are cultured with the cell products harvested from the overlying endodermal cells. This experiment can be performed with different mesenchymal cells to demonstrate the cell differentiation capability of OP-1 morphogen analog in different tissues.

As another example of morphogen-induced cell differentiation, the ability of morphogen analogs to induce osteoblast differentiation can be demonstrated in vitro using primary osteoblast cultures, or osteoblast-like cells lines, and assaying for a variety of bone cell markers that are specific markers for the differentiated osteoblast phenotype, e.g., alkaline phosphatase activity, parathyroid hormone-mediated cyclic AMP (cAMP) production, osteocalcin synthesis, and enhanced mineralization rates.

(2) Induction of Alkaline Phosphatase Activity in Osteoblasts

Cultured osteoblasts in serum-free medium are incubated with a range of morphogen analog concentrations, for example, 0.1, 1.0, 10.0, 40.0 or 80.0 ng morphogen analog/ml medium; or with a similar concentration range of natural OP-1 or TGF-β. After a 72-hour incubation the cell layer is extracted with 0.5 ml of 1% Triton X-100. The resultant cell extract is centrifuged, and 100 ml of the extract is added to 90 ml of para-nitroso-phenylphosphate (PNPP)/glycerine mixture and incubated for 30 minutes in a 37° C. water bath and the reaction stopped with 100 ml NaOH. The samples then are run through a plate reader (e.g., Dynatech MR700 plate reader, and absorbance measured at 400 nm, using p-nitrophenol as a standard) to determine the presence and amount of alkaline phosphate activity. Protein concentrations are determined by the BioRad method. Alkaline phosphatase activity is calculated in units/mg protein, where 1 unit=1 nmol p-nitrophenol liberated/30 minutes at 37° C.

It is contemplated that a morphogen analog, like natural OP-1, will stimulate the production of alkaline phosphatase in osteoblasts thereby promoting the growth and expression of the osteoblast differentiated phenotype. The long term effect of morphogen analog on the production of alkaline phosphatase by rat osteoblasts also can be demonstrated as follows.

Rat osteoblasts are prepared and cultured in multi-well plates as described above. In this example six sets of 24 well plates are plated with 50,000 rat osteoblasts per well. The wells in each plate, prepared as described above, then are divided into three groups: (1) those which receive, for example, 1 ng of morphogen analog per ml of medium; (2) those which receive 40 ng of morphogen analog per ml of medium; and (3) those which receive 80 ng of morphogen analog per ml of medium. Each plate then is incubated for different lengths of time: 0 hours (control time), 24 hours, 48 hours, 96 hours, 120 hours and 144 hours. After each incubation period, the cell layer is extracted with 0.5 ml of 1% Triton X-100. The resultant cell extract is centrifuged, and alkaline phosphatase activity determined using para-nitroso-phenylphosphate (PNPP), as above. It is contemplated that the morphogen analog, like natural OP-1, will stimulate the production of alkaline phosphatase in osteoblasts in a dose-dependent manner so that increasing doses of morphogen analog will further increase the level of alkaline phosphatase production. Moreover, it is contemplated that the morphogen analog-stimulated elevated levels of alkaline phosphatase in the treated osteoblasts will last for an extended period of time.

(3) Induction of PTH-Mediated cAMP

This experiment is designed to test the effect of morphogen analogs on parathyroid hormone-mediated cAMP production in rat osteoblasts in vitro. Briefly, rat osteoblasts are prepared and cultured in a multiwell plate as described above. The cultured cells then are divided into four groups: (I) wells which receive, for example, 1.0, 10.0 and 40.0 ng morphogen analog/ml medium); (2) wells which receive for example, natural OP-1, at similar concentration ranges; (3) wells which receive for example, TGF-β, at similar concentration ranges; and (4) a control group which receives no growth factors. The plate then is incubated for another 72 hours. At the end of the 72 hours the cells are treated with medium containing 0.5% bovine serum albumin (BSA) and 1 mM 3-isobutyl-1-methylxanthine for 20 minutes followed by the addition into half of the wells of human recombinant parathyroid hormone (hPTH, Sigma, St. Louis) at a concentration of 200 ng/ml for 10 minutes. The cell layer then is extracted from each well with 0.5 ml of 1% Triton X-100. The cAMP levels then are determined using a radioimmunoassay kit (e.g., Amersham, Arlington Heights, Ill.). It is contemplated that a morphogen analog alone, like OP-1, will stimulate an increase in the PTH-mediated cAMP response, thereby promoting the growth and expression of the osteoblast differentiated phenotype.

(4) Induction of Osteocalcin Production

Osteocalcin is a bone-specific protein synthesized by osteoblasts which plays an integral role in the rate of bone mineralization in vivo. Circulating levels of osteocalcin in serum are used as a marker for osteoblast activity and bone formation in vivo. Induction of osteocalcin synthesis in osteoblast-enriched cultures can be used to demonstrate morphogen analog efficacy in vitro.

Rat osteoblasts are prepared and cultured in a multi-well plate as above. In this experiment the medium is supplemented with 10% FBS, and on day 2, cells are fed with fresh medium supplemented with fresh 10 mM β-glycerophosphate (Sigma, Inc.).

Beginning on day 5 and twice weekly thereafter, cells are fed with a complete mineralization medium containing all of the above components plus fresh L-(+)-ascorbate, at a final concentration of 50 mg/ml medium. Morphogen analog then is added to the wells directly, e.g., in 50% acetonitrile (or 50% ethanol) containing 0.1% trifluoroacetic acid (TFA), at no more than 5 ml morphogen analog/ml medium. Control wells receive solvent vehicle only. The cells then are re-fed and the conditioned medium sample diluted 1:1 in standard radioimmunoassay buffer containing standard protease inhibitors and stored at −20° C. until assayed for osteocalcin. Osteocalcin synthesis is measured by standard radioimmunoassay using a commercially available osteocalcin-specific antibody.

Mineralization is determined on long term cultures (13 day) using a modified von Kossa staining technique on fixed cell layers: cells are fixed in fresh 4% paraformaldehyde at 23° C. for 10 min, following rinsing cold 0.9% NaCl. Fixed cells then are stained for endogenous alkaline phosphatase at pH 9.5 for 10 min, using a commercially available kit (Sigma, Inc.). Purple stained cells then are dehydrated with methanol and air-dried. After 30 min incubation in 3% AgNO₃ in the dark, H₂O-rinsed samples are exposed for 30 sec to 254 nm UV light to develop the black silver-stained phosphate nodules. Individual mineralized foci (at least 20 mm in size) are counted under a dissecting microscope and expressed as nodules/culture.

It is contemplated that the morphogen analog, like natural OP-1, will stimulate osteocalcin synthesis in osteoblast cultures. Furthermore, it is contemplated that the increased osteocalcin synthesis in response to morphogen analog will be in a dose dependent manner thereby showing a significant increase over the basal level after 13 days of incubation. Enhanced osteocalcin synthesis also can be confirmed by detecting the elevated osteocalcin mRNA message (20-fold increase) using a rat osteocalcin-specific probe. In addition, the increase in osteocalcin synthesis correlates with increased mineralization in long term osteoblast cultures as determined by the appearance of mineral nodules. It is contemplated also that morphogen analog, like natural OP-1, will increase significantly the initial mineralization rate as compared to untreated cultures.

(5) Morphogen-Induced CAM Expression

Members of the BMP/OP family (see FIG. 6) induce CAM expression, particularly N-CAM expression, as part of their induction of morphogenesis (see copending U.S. Ser. No. 922,813). CAMs are morphoregulatory molecules identified in all tissues as an essential step in tissue development. N-CAMs, which comprise at least 3 isoforms (N-CAM-180, N-CAM-140 and N-CAM-120, where “180”, “140” and “120” indicate the apparent molecular weights of the isoforms as measured by SDS polyacrylamide gel electrophoresis) are expressed at least transiently in developing tissues, and permanently in nerve tissue. Both the N-CAM-180 and N-CAM-140 isoforms are expressed in both developing and adult tissue. The N-CAM-120 isoform is found only in adult tissue. Another neural CAM is L1.

The ability of morphogen analogs to stimulate CAM expression may be demonstrated using the following protocol, using NG108-15 cells. NG108-15 is a transformed hybrid cell line (neuroblastoma x glioma, America Type Culture Collection (ATCC), Rockville, Md.), exhibiting a morphology characteristic of transformed embryonic neurons. As described in Example D, below, untreated NG108-15 cells exhibit a fibroblastic, or minimally differentiated, morphology and express only the 180 and 140 isoforms of N-CAM normally associated with a developing cell. Following treatment with members of the vg/dpp subgroup these cells exhibit a morphology characteristic of adult neurons and express enhanced levels of all three N-CAM isoforms.

In this example, NG108-15 cells are cultured for 4 days in the presence of increasing concentrations of either the morphogen analog or natural OP-1 using standard culturing procedures, and standard Western blots are performed on whole cell extracts. N-CAM isoforms are detected with an antibody which crossreacts with all three isoforms, mAb H28.123, obtained from Sigma Chemical Co., St. Louis, the different isoforms being distinguishable by their different mobilities on an electrophoresis gel. Control NG108-15 cells (untreated) express both the 140 kDa and the 180 kDa isoforms, but not the 120 kDa, as determined by Western blot analyses using up to 100 mg of protein. It is contemplated that treatment of NG108-15 cells with morphogen analog, like natural OP-1 may result in a dose-dependent increase in the expression of the 180 kDa and 140 kDa isoforms, as well as the induction of the 120 kDa isoform. In addition, it is contemplated that the morphogen analog, like natural OP-1-induced CAM expression may correlate with cell aggregation, as determined by histology.

(6) OP-1 Morphogen Analog-Induced Redifferentiation of Transformed Phenotype

It is contemplated that a morphogen analog, like natural OP-1, also induces redifferentiation of transformed cells to a morphology characteristic of untransformed cells. The examples provided below detail morphogen-induced redifferentiation of a transformed human cell line of neuronal origin (NG 108-15); as well as mouse neuroblastoma cells (N1E-1 15), and human embryo carcinoma cells, to a morphology characteristic of untransformed cells.

As described above, NG 108-15 is a transformed hybrid cell line produced by fusing neuroblastoma x glioma cells (obtained from ATCC, Rockville, Md.), and exhibiting a morphology characteristic of transformed embryonic neurons, e.g., having a fibroblastic morphology. Specifically, the cells have polygonal cell bodies, short, spike-like processes and make few contacts with neighboring cells. Incubation of NG108-15 cells, cultured in a chemically defined, serum-free medium, with 0.1 to 300 ng/ml of morphogen analog or natural OP-1 for four hours induces an orderly, dose-dependent change in cell morphology.

For example, NG108-15 cells are subcultured on poly L-lysine coated 6 well plates. Each well contains 40-50,000 cells in 2.5 ml of chemically defined medium. On the third day, 2.5 ml of morphogen analog or natural OP-1 in 60% ethanol containing 0.025% trifluoroacetic is added to each well. The media is changed daily with new aliquots of morphogen. It is contemplated that morphogen analog, like OP-1, may induce a dose-dependent redifferentiation of the transformed cells, including a rounding of the soma, an increase in phase brightness, extension of the short neurite processes, and other significant changes in the cellular ultrastructure. After several days it is contemplated also that treated cells may begin to form epithelioid sheets that then become highly packed, multi-layered aggregates, as determined visually by microscopic examination.

Moreover, it is contemplated that the redifferentiation may occur without any associated changes in DNA synthesis, cell division, or cell viability, making it unlikely that the morphologic changes are secondary to cell differentiation or a toxic effect of the morphogen. In addition, it is contemplated that the morphogen analog-induced redifferentiation may not inhibit cell division, as determined by ³H-thymidine uptake, unlike other molecules such as butyrate, DMSO, retinoic acid or forskolin, which have been shown to stimulate differentiation of transformed cells in analogous experiments. Thus, it is contemplated that the morphogen analog, like natural OP-1, may maintain cell stability and viability after inducing redifferentiation.

The morphogens described herein would, therefore, provide useful therapeutic agents for the treatment of neoplasias and neoplastic lesions of the nervous system, particularly in the treatment of neuroblastomas, including retinoblastomas, and gliomas.

(7) BMP Induced Dendritic Growth

BMP family members can induce dendritic growth in cultures of both peripheral and central nervous system neurons. For example, recombinant human OP-1, BMP-6, BMP-2, and Drosophila 60A induce dendritic growth in cultures of rat sympathetic neurons (Guo et al. (1998) Neuroscience Letter 245: 131). More recently, Beck et al. has demonstrated that BMP-5 can also induce dendritic growth in sympathetic neurons, and has additionally shown that BMP antagonists such as noggin and follistatin inhibit dendritic growth in these cultures (Beck et al. (2001) BMC Neuroscience 2: 12). Similar experiments have demonstrated that BMPs stimulate dendritic growth in neurons derived from the central nervous system (Le Roux et al. (1999) Experimental Neurology 160: 151; Withers et al. (2000) European Journal of Neuroscience 12: 106).

The induction of dendritic growth in cultures of peripheral or central nervous system neurons can be used to assay the activity of BMP analogs of the present invention. Neurons are cultured in the presence of one or more morphogen analogs, and dendrite growth is compared to neurons cultured in the absence of the morphogen analog. Dendrite growth is assessed by visual inspection of the cultures including inspection of dendrite length and branching. In addition to visual inspection of dendrite morphology, cultures can be analyzed for the expression of molecular markers indicative of dendrite formation such as MAP2. It is contemplated that a morphogen analog will induce dendrite growth in cultures of peripheral or central nervous system neurons. It is further contemplated that a morphogen analog will increase the expression of molecular markers of dendrites such as MAP2.

Additionally, the induction of dendritic growth can be used to assay the activitiy of BMP antagonists. Neurons derived from the central or peripheral nervous system are cultured in the presence of a BMP family member known to promote dendrite growth, such as OP-1. Dendrite growth in such cultures is compared to dendrite growth in neurons cultured in the presence of both the BMP family member and one or more putative BMP antagonist. An analog is identified as a BMP antagonist if it can reduce or inhibit dendrite growth in response to a BMP family member. As discussed in detail above, dendrite growth is assessed by visual inspection of dendrite length and branching, and/or by the expression of dendrite specific markers such as MAP2. It is contemplated that a morphogen antagonist will reduce or inhibit dendrite growth in neurons derived from the central or peripheral nervous system. It is further contemplated that a morphogen antagonist will reduce or inhibit expression of molecular markers of dendrites such as MAP2.

(E) Maintenance of Phenotype

Morphogen analogs, like natural OP-1, also may be used to maintain a cell's differentiated phenotype. This application is particularly useful for inducing the continued expression of phenotype in senescent or quiescent cells.

(1) In vitro Model for Phenotypic Maintenance

The phenotypic maintenance capability of morphogens is determined readily. A number of differentiated cells become senescent or quiescent after multiple passages in vitro under standard tissue culture conditions well described in the art (e.g., Culture of Animal Cells: A Manual of Basic Techniques, C. R. Freshney, ed., Wiley, 1987). However, if these cells are cultivated in vitro in association with a morphogen such as OP-1, cells are stimulated to maintain expression of their phenotype through multiple passages. For example, the alkaline phosphatase activity of cultured osteoblasts, such as cultured osteosarcoma cells and calvaria cells, is significantly reduced after multiple passages in vitro. However, if the cells are cultivated in the presence of OP-1, alkaline phosphatase activity is maintained over extended periods of time. Similarly, phenotypic expression of myocytes also is maintained in the presence of a morphogen. In the experiment, osteoblasts are cultured as described in Example A. The cells are divided into groups, incubated with varying concentrations of either morphogen analog or natural OP-1 (e.g., 0-300 ng/ml) and passaged multiple times (e.g., 3-5 times) using standard methodology. Passaged cells then are tested for alkaline phosphatase activity, as described in Example C as an indication of differentiated cell metabolic function. It is contemplated that osteoblasts cultured in the absence of morphogen analog may have reduced alkaline phosphatase activity, as compared to morphogen analog, or natural OP-1-treated cells.

(2) In vivo Model-for Phenotypic Maintenance.

Phenotypic maintenance capability also may be demonstrated in vivo, using a standard rat model for osteoporosis. Long Evans female rats (Charles River Laboratories, Wilmington, Mass.) are sham-operated (control animals) or ovariectomized using standard surgical techniques to produce an osteoporotic condition resulting from decreased estrogen production. Following surgery, e.g., 200 days after ovariectomy, rats are systemically provided with phosphate buffered saline (PBS) or morphogen, (e.g., morphogen analog, or natural OP-1, 1-100 mg) for 21 days (e.g., by daily tail vein injection.) The rats then are sacrificed and serum alkaline phosphatase levels, serum calcium levels, and serum osteocalcin levels are determined, using standard methodologies as described therein and above. It is contemplated that the morphogen analog treated rats, like the OP-1 treated rats may exhibit elevated levels of osteocalcin and alkaline phosphatase activity. It is contemplated also that histomorphometric analysis on the tibial diaphyseal bone may show improved bone mass in morphogen analog-treated animals as compared with untreated, ovariectomized rats.

(3) Maintainence of Smooth Muscle Cell Phenotype

The vasculature is a crucial component of virtually all other systems in both developing and adult organisms. An important aspect of development that must continue throughout the life of all organisms is the maintainence of the smooth muscle cell phenotype of the vasculature, and failure to do this results in a host of vasculature proliferative disorders.

BMP family members have been shown to maintain the smooth muscle cell phenotype in human primary aortic smooth muscle cells in culture (Dorai et al. (2000) Journal of Cell Physiology 184: 37; Dorai and Sampath (2001) Journal Bone Joint Surg Am 83-A: S70). These results not only suggest that morphogen analogs may be useful in treating proliferative vasculature disorders, but also provide a useful assay for evaluating the morphogen analogs of the present invention.

Primary human aortic smooth muscle (HASM) cells are cultured in the presence of serum or a growth factor such as PDGF. Such culture conditions have been previously shown to induce proliferation of HASM cells. The addition of a BMP, such as OP-1, inhibits serum and growth factor induced proliferation and stimulates the expression of smooth muscle cell specific markers. One or more BMP analogs can be added to HASM cultures which have been induced to proliferate. Proliferation of HASM cells in the presence of the BMP analog is assayed and compared to proliferation in the absence of the BMP analog. Proliferation is assayed by any of a number of methods well known in the art including cell counting and uptake of ³H-thymidine. It is contemplated that a BMP analog will decrease cell proliferation in smooth muscle cells induced to proliferate by growth factor or serum stimulation. Furthermore, it is contemplated that a BMP analog will stimulate expression of smooth muscle cell specific markers. Exemplary smooth muscle specific markers include α-actin and smooth muscle cell specific myosin heavy chain, and the expression of these markers is assayed by any one of a number of methods well known in the art including Northern blot, RT-PCR, and in situ hybridization.

F. Proliferation of Progenitor Cell Populations

Progenitor cells may be stimulated to proliferate in vivo or ex vivo. It is contemplated that cells may be stimulated in vivo by injecting or otherwise providing a sterile preparation containing the morphogen analog into the individual. For example, the hematopoietic pluripotential stem cell population of an individual may be stimulated to proliferate by injecting or otherwise providing an appropriate concentration of morphogen analog to the individual's bone marrow.

Progenitor cells may be stimulated ex vivo by contacting progenitor cells of the population to be enhanced with a morphogenically active morphogen analog under sterile conditions at a concentration and for a time sufficient to stimulate proliferation of the cells. Suitable concentrations and stimulation times may be determined empirically, essentially following the procedure described in Example A, above. It is contemplated that a morphogen analog concentration of between about 0.1-100 ng/ml and a stimulation period of from about 10 minutes to about 72 hours, or, more generally, about 24 hours, typically should be sufficient to stimulate a cell population of about 104 to 106 cells. The stimulated cells then may be provided to the individual as, for example, by injecting the cells to an appropriate in vivo locus. Suitable biocompatible progenitor cells may be obtained by any of the methods known in the art or described hereinabove.

G. Regeneration of Damaged or Diseased Tissue

It is contemplated that morphogen analogs may be used to repair diseased or damaged mammalian tissue. The tissue to be repaired preferably is assessed first, and excess necrotic or interfering scar tissue removed as needed, e.g., by ablation or by surgical, chemical, or other methods known in the medical arts.

Morphogen analog then may be provided directly to the tissue locus as part of a sterile, biocompatible composition, either by surgical implantation or injection. The morphogen analog also may be provided systemically, as by oral or parenteral administration. Alternatively, a sterile, biocompatible composition containing progenitor cells stimulated by a morphogenically active morphogen analog may be provided to the tissue locus. The existing tissue at the locus, whether diseased or damaged, provides the appropriate matrix to allow the proliferation and tissue-specific differentiation of progenitor cells. In addition, a damaged or diseased tissue locus, particularly one that has been further assaulted by surgical means, provides a morphogenically permissive environment.

Systemic provision of morphogen analog may be sufficient for certain applications (e.g., in the treatment of osteoporosis and other disorders of the bone remodeling cycle).

In some circumstances, particularly where tissue damage is extensive, the tissue may not be capable of providing a sufficient matrix for cell influx and proliferation. In these instances, it may be necessary to provide progenitor cells stimulated by the morphogen analog to the tissue locus in association with a suitable, biocompatible, formulated matrix, prepared by any of the means described below. The matrix preferably is in vivo biodegradable. The matrix also may be tissue-specific and/or may comprise porous particles having dimensions within the range of 70-850 μm, most preferably 150-420 μm.

A morphogen analog also may be used to prevent or substantially inhibit immune/inflammatory response-mediated tissue damage and scar tissue formation following an injury. A morphogen analog may be provided to a newly injured tissue locus, to induce tissue morphogenesis at the locus, preventing the aggregation of migrating fibroblasts into non-differentiated connective tissue.

Preferably the morphogen analog may be provided as a sterile pharmaceutical preparation injected into the tissue locus within five hours of the injury. Where an immune/inflammatory response is unavoidably or deliberately induced, as part of, for example, a surgical or other aggressive clinical therapy, a morphogen analog preferably may be provided prophylactically to the patient prior to, or concomitant with, the therapy.

H. Reporter Assay

Agonists and antagonists can be rapidly screened in vitro using a reporter based assay. The assay can be performed using any BMP responsive cell or cell line. Given the well established involvement of TGFβ superfamily members generally, and BMPs specifically, in the development of tissues derived from all three germ layers, a wide range of cells and cell lines can be used to carry out such a reporter based assay. Examples of such cells and cell lines include HeLa cells, NIH3T3 cells, CHO cells, COS cells, HepG2 cells, and derivative thereof. Further examples include primary cell cultures, embryonic stem cells, embryonic germ cells, adult stem cells (including mesechymal stem cells, neural stem cells, hematopoietic stem cells, pancreatic stem cells, cardiac stem cells, and skin-derived stem cells). Still further examples include the cells and cell lines cited in the forgoing examples.

Cells responsive to BMP signaling are transfected with a reporter gene construct consisting of a BMP responsive promoter element linked to a reporter gene. The reporter gene may encode any readily detectable gene product. Exemplary reporter gene products include GFP, or derivatives thereof, luciferase, and β-galactosidase. The BMP responsive promoter element may be all, or a portion, of a promoter element which mediates a change in gene expression in response to BMP signaling. The BMP responsive promoter element may also include multimers of one or more BMP binding elements. Such BMP responsive elements can be readily identified within the promoters of genes regulated by BMP signaling by one skilled in the art using standard techniques in molecular biology. The literature is replete with examples of BMP responsive elements, and the present invention contemplates the use of these or any other BMP responsive element (Rebbert and Dawid (1997) Proc Natl Acad Sci 94: 9717-9722; Jonk et al. (1998) Journal of Biological Chemistry 273: 21145-21152; Ishida et al. (2000) Journal of Biological Chemistry 275: 6075-6079; Henningfeld et al. (2000) Journal of Biological Chemistry 275: 21827-21835; Yeh and Lee (2000) Endocrinology 141: 3278-3286; Lopez-Rovira et al. (2002) Journal of Biological Chemistry 277: 3176-3185).

Cells transfected with the reporter gene construct are contacted with an agonist or antagonist, and expression of the reporter gene is assayed. An analog is an agonist if it increases expression of the reporter gene in comparison to the expression of the reporter gene in the absence of said agonist, and/or in comparison to the expression of the reporter gene following contacting the cells with a BMP protein. An analog is an antagonist if it decreases expression of the reporter gene in comparison to the expression of the reporter gene in the absence of said antagonist, and/or in comparison to the expression of the reporter gene following contacting the cells with a BMP protein.

The above reporter based assay can be easily adapted for high-throughput analysis of agonists and antagonists using standard techniques.

Described below is a protocol for demonstrating whether a morphogen analog induces tissue morphogenesis in bone.

(1) Morphogen Analog-Induced Bone Morphogenesis.

A particularly useful mammalian tissue model system for demonstrating and evaluating the morphogenic activity of a morphogen analog is the endochondral bone tissue morphogenesis model known in the art and described, for example, in U.S. Pat. No. 4,968,590, incorporated herein by reference. The ability to induce endochondral bone formation includes the ability to induce proliferation and differentiation of progenitor cells into chondroblasts and osteoblasts, the ability to induce cartilage matrix formation, cartilage calcification, and bone remodeling, and the ability to induce formation of an appropriate vascular supply and hematopoietic bone marrow differentiation.

The local environment in which the morphogenic material is placed is important for tissue morphogenesis. As used herein, “local environment” is understood to include the tissue structural matrix and the environment surrounding the tissue. For example, in addition to needing an appropriate anchoring substratum for their proliferation, the cells stimulated by morphogens need signals to direct the tissue-specificity of their differentiation. These signals vary for the different tissues and may include cell surface markers. In addition, vascularization of new tissue requires a local environment that supports vascularization.

The following sets forth various procedures for evaluating the in vivo morphogenic utility of morphogen analogs and morphogen analog-containing compositions. The compositions may be injected or surgically implanted in a mammal, following any of a number of procedures well known in the art. For example, surgical implant bioassays may be performed essentially following the procedure of Sampath et al. (1983) Proc. Natl. Acad. Sci. USA 80: 6591-6595 and U.S. Pat. No. 4,968,590.

Histological sectioning and staining is preferred to determine the extent of morphogenesis in vivo, particularly in tissue repair procedures. Excised implants are fixed in Bouins Solution, embedded in paraffin, and cut into 6-8 μm sections. Staining with toluidine blue or hemotoxylin/eosin demonstrates clearly the ultimate development of the new tissue. Twelve-day implants are usually sufficient to determine whether the implants contain newly induced tissue.

Successful implants exhibit a controlled progression through the stages of induced tissue development allowing one to identify and follow the tissue-specific events that occur. For example, in endochondral bone formation the stages include: (1) leukocytes on day one; (2) mesenchymal cell migration and proliferation on days two and three; (3) chondrocyte appearance on days five and six; (4) cartilage matrix formation on day seven; (5) cartilage calcification on day eight; (6) vascular invasion, appearance of osteoblasts, and formation of new bone on days nine and ten; (7) appearance of osteoclastic cells, and the commencement of bone remodeling and dissolution of the implanted matrix on days twelve to eighteen; and (8) hematopoietic bone marrow differentiation in the resulting ossicles on day twenty-one.

In addition to histological evaluation, biological markers may be used as markers for tissue morphogenesis. Useful markers include tissue-specific enzymes whose activities may be assayed (e.g., spectrophotometrically) after homogenization of the implant. These assays may be useful for quantitation and for rapidly obtaining an estimate of tissue formation after the implants are removed from the animal. For example, alkaline phosphatase activity may be used as a marker for osteogenesis.

Incorporation of systemically provided morphogen analog may be followed using labeled protein (e.g., radioactively labeled) and determining its localization in the new tissue, and/or by monitoring their disappearance from the circulatory system using a standard labeling protocol and pulse-chase procedure. A morphogen analog also may be provided with a tissue-specific molecular tag, whose uptake may be monitored and correlated with the concentration of morphogen analog provided. As an example, ovary removal in female rats results in reduced bone alkaline phosphatase activity, and renders the rats predisposed to osteoporosis (as described in Example E). If the female rats now are provided with morphogen analog, a reduction in the systemic concentration of calcium may be seen, which correlates with the presence of the provided morphogen analog and which is anticipated to correspond with increased alkaline phosphatase activity.

### Example 2

Biological Activity of Finger 1, Finger 2, and Heel Peptides

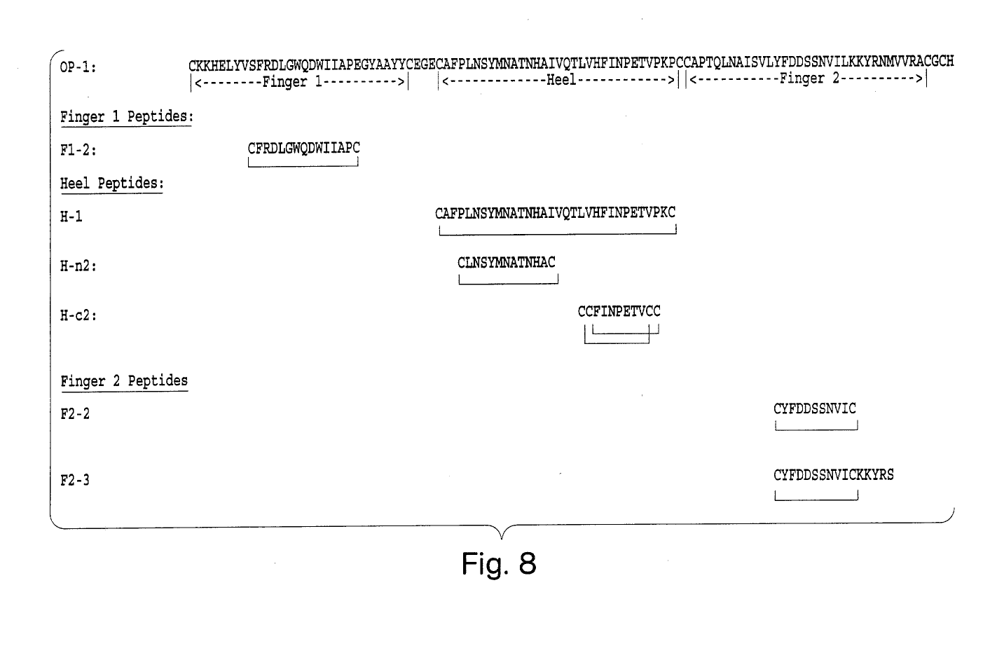

The hOP-1-based peptides described in this example (FIG. 8) were produced and characterized prior to determination of the three-dimensional structure of hOP-1. These peptides either agonize or antagonize the biological activity of hOP-1. It is contemplated that, further refinements based upon the hOP-1 crystal structure, for example, the choice of more suitable sites for cyclizing peptides which constrain the peptide into a conformation that more closely mimics the shape of the corresponding region in hOP-1, may be used to further enhance the agonistic or antagonistic properties of such hOP-1-based peptides.

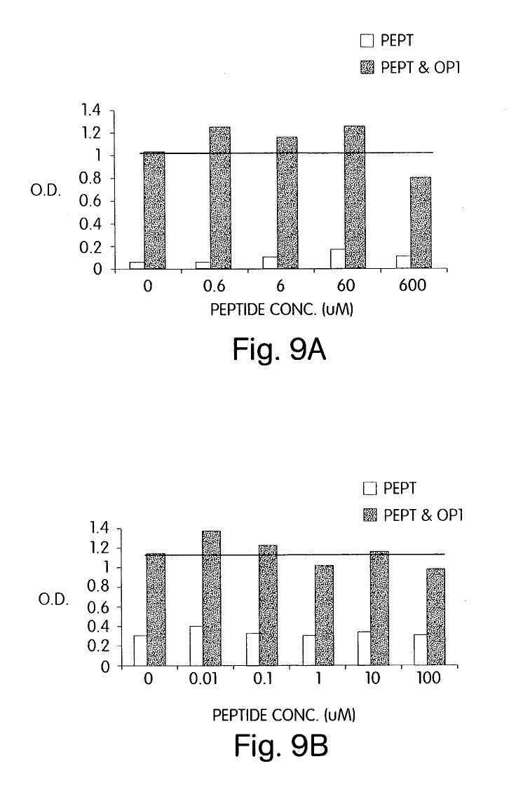

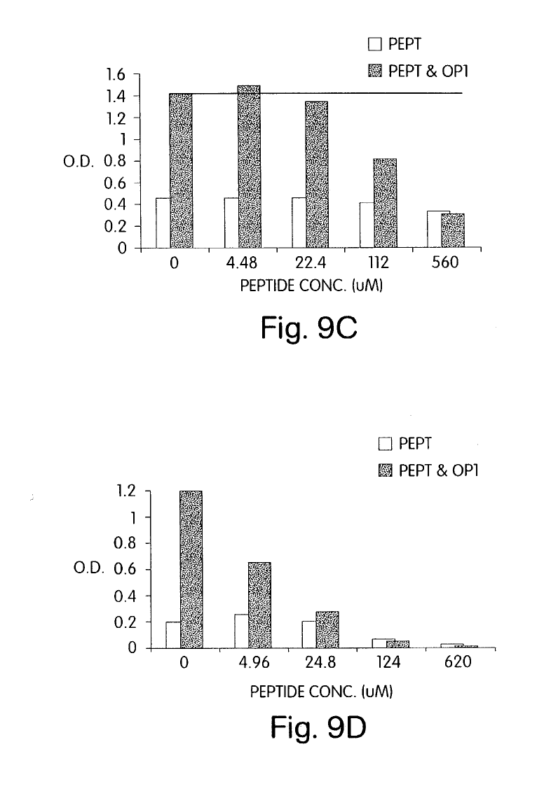

All of the peptides used in the following experiments, as well as their relationships with the mature hOP-1 amino acid sequence, are shown in FIG. 9. The finger 1-based peptides are designated F1-2 (SEQ ID No. 6); the heel-based peptides are designated H-1, H-n2 and H-c2 (SEQ ID Nos. 11-13, respectively); and the finger 2-based peptides are designated F2-2 and F2-3 (SEQ ID Nos. 17 and 19, respectively). Potential intra-peptide disulfide linkages are shown for each peptide. All the peptides were synthesized on a standard peptide synthesizer in accordance with the manufacturer's instructions. The peptides were deprotected, cyclized by oxidation, and then cleaved from resin prior to use.

In a first series of experiments, increasing concentrations of peptides F2-2 (FIG. 9A), F2-3 (FIG. 9B), Hn-2 (FIG. 9C) and Hc-2 (FIG. 9D) were added to ROS cells either alone (open bars) or in combination with 40 ng/ml soluble OP-1 (filled bars) and their effect on alkaline phosphatase activity measured. Soluble OP-1 is the form of OP-1 in which the pro-domain is still attached to the mature portion of OP-1 (see WO 94/03600). A basal alkaline phosphatase activity is shown by the line and represents the alkaline phosphatase activity of cells incubated in the absence of both soluble OP-1 and peptide.

In FIG. 9A, peptide F2-2 at a concentration of about 60 μM appears to double the basal alkaline phosphatase level and, in the presence of soluble OP-1, increases alkaline phosphatase activity by about 20% relative to soluble OP-1 alone. In FIG. 9B, peptide F2-3 at a concentration of about 0.01 μM appears to increase the basal alkaline phosphatase level and, in the presence of soluble OP-1, increases alkaline phosphatase activity by about 20% relative to soluble OP-1 alone. Accordingly, both peptides F2-2 and F2-3, in the alkaline phosphatase assay, appear to act as weak OP-1 agonists.

In FIG. 9C, peptide H-n2 displays little or no effect on alkaline phosphatase activity either alone or in combination with soluble OP-1. FIG. 9D, peptide H-c2, at concentrations greater than about 5 μM, appears to antagonize the activity of soluble OP-1.

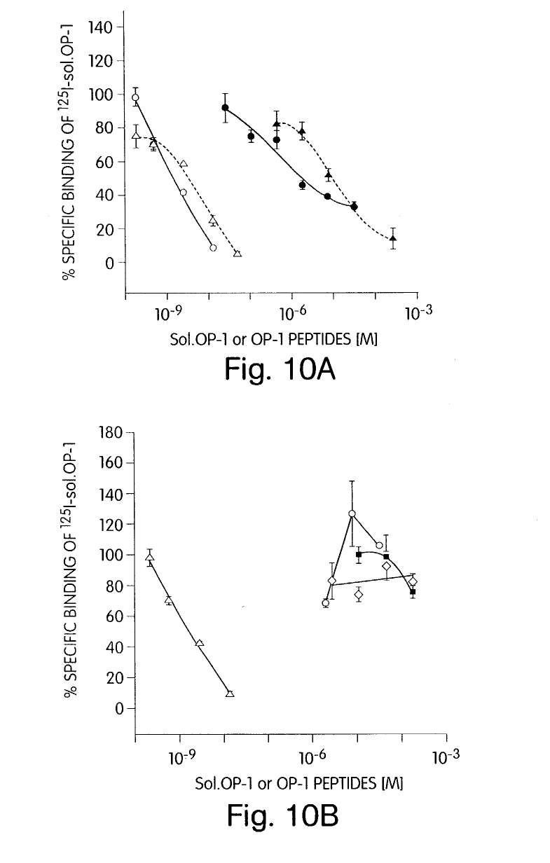

In a second series of experiments, the ability of unlabeled soluble OP-1 and unlabeled peptides F1-2, F2-2, F2-3, H-n2 and H-c2 to displace ¹²⁵I labeled soluble OP-1 from ROS cell membranes was measured. The activities of peptides F2-2 and F2-3 relative to soluble OP-1 are shown in FIG. 10A, and the activities of peptides Fi-2, H-n2 and H-c2 relative to soluble OP-1 are shown in FIG. 10B. OP-1 receptor-enriched plasma membranes of ROS cells were incubated for 20 hrs at 4° C. with ¹²⁵I-labeled soluble OP-1 and unlabeled peptide. Receptor bound material was separated from unbound material by centrifugation at 39,500×g. The resulting pellet was harvested and washed with 50 mM HEPES buffer, pH 7.4 containing 5 mM MgCl₂ and 1 mM CaCl₂ Radioactivity remaining in the pellet was determined by means of a gamma counter.

In FIG. 10A, peptide F2-2 (filled circles) soluble competes with soluble OP-1 with an Effective Dose₅₀ (ED₅₀) of about 1 μM, but cannot completely displace soluble OP-1 ED₅₀ is the concentration of peptide to produce half maximal displacement of labeled soluble OP-1. Peptide F2-3 (filled triangles) competes and is able to completely displace soluble OP-1 with an ED₅₀ of about 5 μM. In FIG. 10B, peptide F1-2 (filled boxes), peptide H-n2 (open diamonds) and peptide H-c2 (open circles) all appear to exhibit little or no ability to displace iodinated soluble OP-1 from ROS cell membranes.

Although the peptide experiments appear promising, it is contemplated that resolution of the hOP-1 structure will enable the skilled practitioner to design constrained peptides that more closely mimic the receptor-binding domains of human OP-1 and which are more effective at agonizing or antagonizing an hOP-1 mediated biological effect.

### Example 3

In order to gain insights into what portions of OP-1 contribute to its biological activity, two different approaches were used, one to examine the N-terminus extension, and the other to analyze different regions of 7-CYS domain of OP-1.

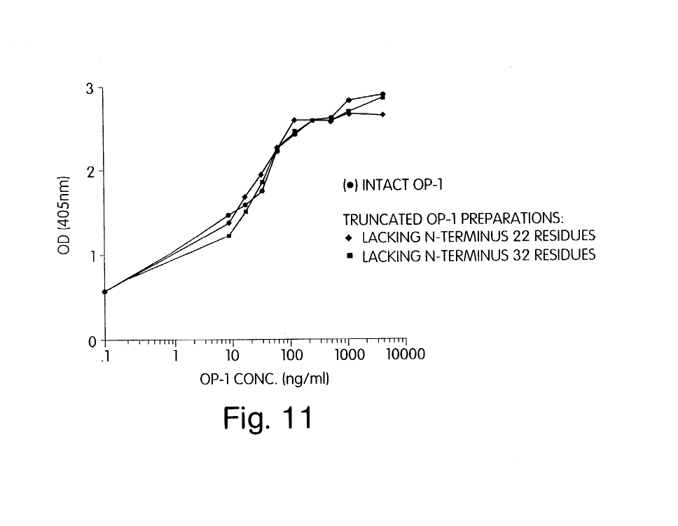

N-terminus extension: In the first approach, highly purified truncated OP-1 preparations lacking N-terminus Ser¹-Arg²² or Ser¹-Ser³² residues were analyzed by an in vitro bio-assay to determine the ability of these preparations to induce ROS cell responsiveness in terms of an increase in cellular alkaline phosphatase activity. In this assay, the two truncated preparations lacking the N-terminus extension, gave dose-related responses identical to that of an intact OP-1 preparation (FIG. 11). The fact that the truncated OP-1 preparations were as potent as that of intact OP-1 strongly suggests that the N-terminus extension is not required for the biological activity of OP-1.

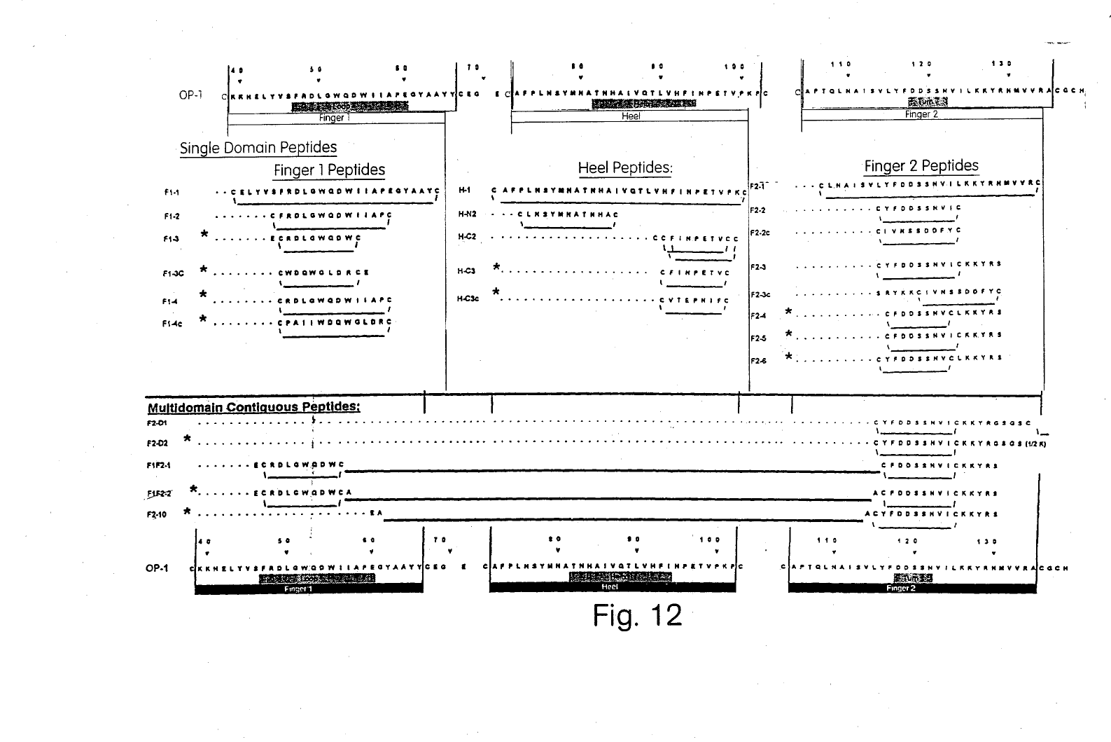

7-Cys domain: In the second approach, we have used a widely accepted synthetic peptide approach to map the regions of Cys domain that are involved in the receptor-binding as well as biological activity of OP-1. Using knowledge of the 3DS of OP-1 and the location of solvent accessible residue positions that are also variable in composition, we have designed a set of 8 peptides. Of these, three peptides covered the complete finger 1 (F1-1), finger 2 (F2-1) and Heel (H-1) regions of the 7 Cys domain (SEQ ID Nos. 5, 16, and 11, respectively). The other 5 peptides covered the accessible loop regions of this domain. FIG. 12 illustrates the sequences, locations and cyclization points of all of the peptides synthesized and analyzed. All peptides were cyclized at the N- and C-termini via a disulfide bond which was engineered so as to disturb neither the anti-parallel beta-sheet structure of the finger regions nor the helical structure of the heel region. The activities of these peptides, reported below, are summarized in Table 5. Based on the OP-1 structure, we have hypothesized that a high affinity receptor binding site of OP-1 comprises either the combined finger 1 and finger 2 regions or two finger 2 loop regions. Thus contiguous peptides F1F2-1 and F2-D1 (SEQ ID Nos. 26 and 24, respectively) were also designed.

Assay of peptides: The receptor-binding activities of OP-1 synthetic peptides were determined by a radio-ligand receptor assay using ¹²⁵I-labeled OP-1 as ligand, and an OP-1 receptor-enriched plasma membrane fraction of ROS cells. The assay is based on competition between radiolabeled OP-1 and unlabeled OP-1 or peptide for binding to membrane-bound receptor. A well characterized ROS cell based in vitro bio-assay was used to examine the effects of OP-1 peptides on basal as well as OP-1 induced cellular alkaline phosphatase activity.

Activity Profiles of OP-1 Peptides:

Finger 1 peptides: Peptide F1-1 (SEQ ID No. 5) covers all of the finger 1 region and has cyclized ends (FIG. 12). When ROS cells were incubated with increasing concentrations of this peptide (2 to 260 μM) together with OP-1 (1.33 nM), the peptide behaved as an antagonist and inhibited OP-1 induced cellular responsiveness in a dose-dependent manner. A 50% inhibition of cellular responsiveness to OP-1 was observed at a peptide concentration of 20 μM. The peptide alone, however, was effective only at higher concentrations (52-260 μM) to inhibit the basal cellular responsiveness (Table 5).

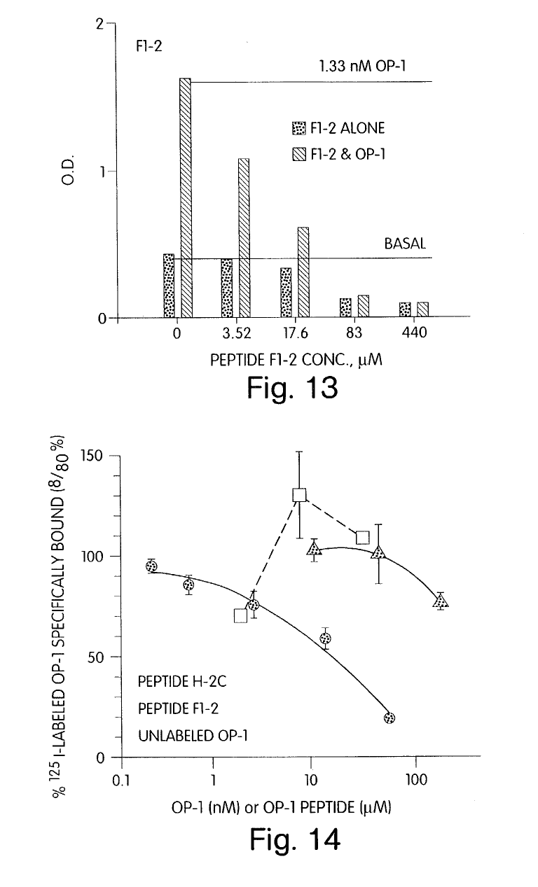

Peptide F1-2 (SEQ ID No. 6) is a 15-residue loop region peptide Fl-i that is cyclized at the ends (FIG. 12). In the ROS cell based bioassay, the peptide behaved similar to F1-1, weakly inhibiting the basal cell responsiveness. When the peptide was tested together with OP-1, it behaved as an antagonist and inhibited the cell responsiveness to OP-1 in a dose-dependent manner (FIG. 13). A 50% inhibition of the cell responsive to OP-1 was observed at a peptide concentration of 10 μM. When this peptide was tested in a radioligand receptor assay, it required relatively higher concentrations (179 μM) to inhibit the binding of ¹²⁵I-labeled OP-1 to the receptor, thus showing a relatively weak activity in the assay (FIG. 14).

Heel peptides: Peptide H-1 (SEQ ID No. 11) covers the whole of the Heel region and is cyclized near the knot region (FIG. 12). In the ROS cell based bioassay, this peptide behaved as a weak antagonist as it required relatively high concentrations (44-220 μM) to inhibit the OP-1 induced cell response. Peptide alone was also effective only at very high concentrations in inhibiting the basal alkaline phosphatase activity of the ROS cell (Table 5).

Peptide H-N2 (SEQ ID No. 12) covers 11 residues (plus Cys) in the loop on the N-terminal end of the Heel helix (H-1) (FIG. 12). The peptide failed to inhibit the binding of ¹²⁵I-labeled OP-1 to the receptor, suggesting a lack of involvement of this region in the recognition of OP-1 by the receptor. Consistent with this, the peptide was not effective in inhibiting either basal or OP-1 induced cellular responsiveness in the ROS cell based bioassay.

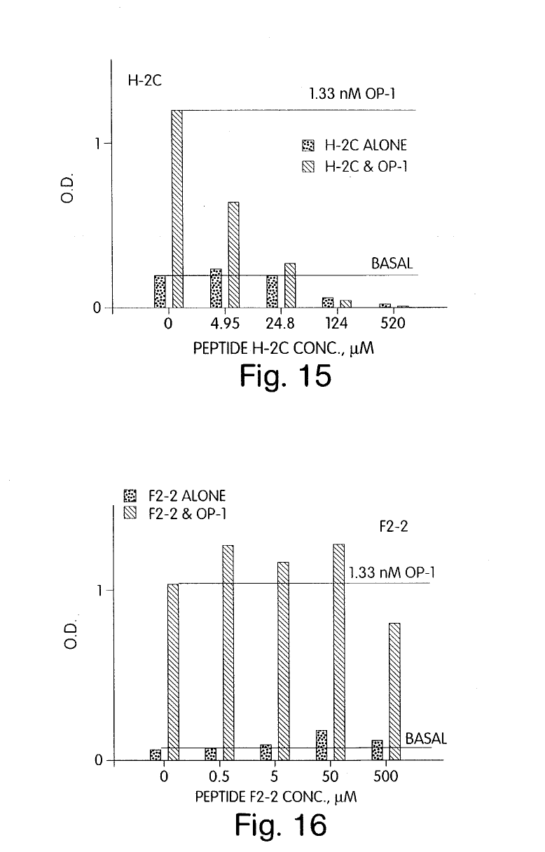

Peptide H-C2 (SEQ ID No. 13) covers 7 residues (plus Cys) in the loop on the C-terminal end of the Heel helix (H-1) (FIG. 12). In the radioligand receptor assay, this peptide at 8 μM concentration potentiated the binding of ¹²⁵I-labeled OP-1 to the receptor (FIG. 14). In ROS cell based bioassay, however, the peptide behaved as an antagonist inhibiting the basal as well as the OP-1 induced cell response. A 50% inhibition of cellular responsiveness to OP-1 was observed at a peptide concentration of 5 μM (FIG. 15).

Finger 2 peptides: Peptide F2-1 (SEQ ID No. 16) covers the entire finger 2 region of OP-1, and is cyclized close to the knot region (FIG. 12). In the ROS cell-based bioassay this peptide effectively inhibited both the basal as well as the OP-1 induced cellular responsiveness in a dose-dependent manner. The peptide, at a concentration of 8 μM, inhibited the OP-1 induced cell response by 50% (Table 5).

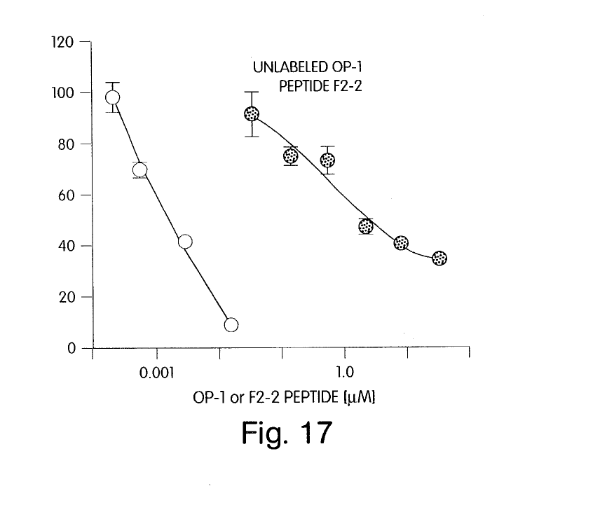

Peptide F2-2 (SEQ ID No. 17) contains 9 residues at the tip of finger 2 including 2 Asp residues; its ends are cyclized (FIG. 12). In the ROS cell based bioassay, the peptide behaved as a weak agonist at lower concentrations (0.6-60 μM), as it slightly potentiated (20%) OP-1 induced cell responsiveness (FIG. 16). At higher concentrations, however, the peptide inhibited the responsiveness to OP-1. When this peptide was tested in a radioligand receptor assay, it effectively inhibited the binding of ¹²⁵I-labeled OP-1 in a dose-dependent manner with an ED₅₀ of 1 μM (FIG. 17).

Peptide F2-2c (SEQ ID No. 18) served as a control peptide in the assays. This is a cyclized peptide having 9 residues identical to those of peptide F2-2, but in reverse order (FIG. 12). In radioligand receptor assay, this peptide was inactive. Also, the peptide has no effect on basal or OP-1 induced ROS cell responsiveness.

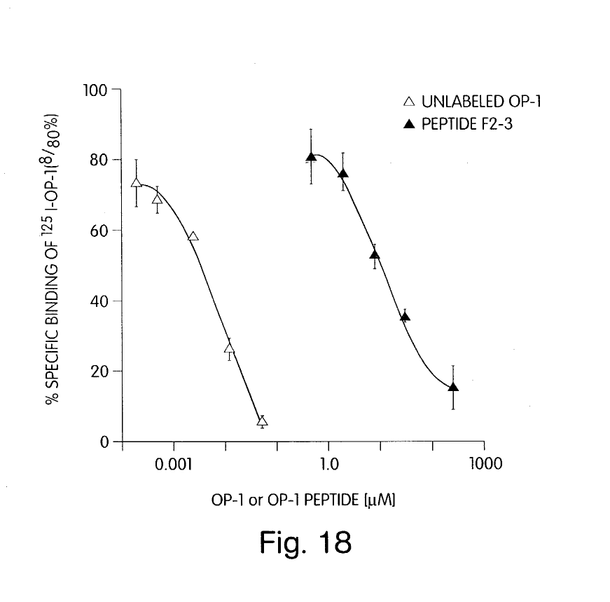

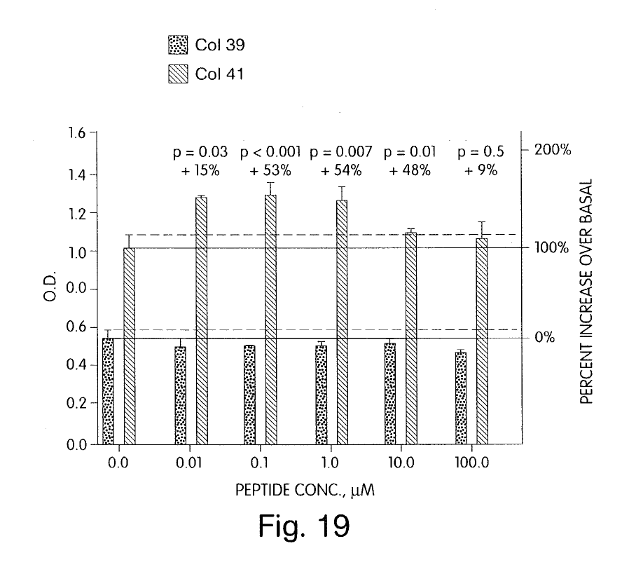

Peptide F2-3 (SEQ ID No. 19) contains 16 residues, of which 11 residues (including two Cys residues) are at the tip of finger 2 and are identical to that of F2-2. In addition, the peptide has a 5-residue C-terminal extension. The peptide is cyclized into an 11-residue loop by an internal Cys replacement plus an N-terminus Cys. It contains 2 negative charges in the finger 2 loop and 3 positive charges in the C-terminus of the tip (FIG. 12). In the radioligand receptor assay, the peptide gave a dose-related inhibition of ¹²⁵I-labeled OP-1 binding to the receptor, with an ED⁵⁰ of 10 μM (FIG. 18). Moreover, the calculated slopes of the dose-response lines for the peptide and a reference preparation unlabeled OP-1 were similar suggesting that the dose-response lines were parallel. In the ROS cell-based bioassay, the peptide had no significant effect on basal cell responsiveness, but behaved in an agonistic manner in the presence of a sub-maximal concentration of OP-1 (1.33 nM). Interestingly, the peptide was extremely effective at low concentrations (0.01-1 μM) where it significantly potentiated OP-1 induced cellular alkaline phosphatase activity (FIG. 19). For peptide concentrations as low as 0.01, 0.1 and 1 ELM, the % increases in OP-1 response observed (relative to the absence of peptide) were 53% (P<0.001), 54%(P=0.007) and 48% (P=0.01), respectively.

Contiguous peptides, F1F2-1 and F2-D1: In order to increase the contacts and thereby increasing the affinity of peptide for the receptor, we have designed two different contiguous peptides. Peptide F1F2-1 was designed to bring together two active regions representing the loops of finger 1 and finger 2. The ends of each loop region were cyclized, and the loops were connected by a peptide bond between the cysteines (FIG. 12). A second peptide, F2-D1, was designed to mimic the bivalent geometry of the finger 2 regions in OP-1 dimer. Essentially, two F2-3 sequences were joined tail-to-tail using a Gly Ser spacer to obtain the necessary separation (FIG. 12). The peptide required two separate oxidations, one to cyclize the loops and the second to form the dimer. Both peptides were synthesized, purified, characterized, and are being analyzed for activity by radioligand receptor assay and ROS cell based bioassay.

Of these peptides, the peptides from the finger 1 region (F1-1, Fi-2) and from the heel region (H-1, H-C2) behaved as antagonists, as these peptides interfered with ¹²⁵I-labeled OP-1 binding to the receptor, and effectively inhibited the OP-1-induced ROS cell responsiveness in terms of increase in cellular alkaline phosphatase activity. On the other hand, peptides from the finger 2 region (F2-2 and F2-3) effectively competed with I-labeled OP-1 at the receptor level, and instead of inhibiting ROS cell responsiveness to OP-1, these peptides, at relatively lower concentrations, were effective at potentiating the cell responsiveness to OP-1. Thus these peptides, upon occupying the receptor, modulated the biological properties of OP-1 and thereby qualify as modulators of OP-1 activity. The peptides H-C2 and F2-3 are judged to be highly active, and are considered to be lead peptides for antagonistic and modulating activities of OP-1, respectively. Because the F2-3 peptide seems to increase the effectiveness of low levels of OP-1 and since OP-1 circulates in the blood and has paracrine/endocrine roles at target tissues, it is possible that F2-3 can function in vivo as an agonist. Although the peptide F1-2 is a relatively weak antagonist, it is highly likely to be a part of the receptor recognition site. Two contiguous peptides, peptides F1F2-1 and F2-D1, have been synthesized to study the agonistic determinants of OP-1, and are currently being tested for activity.

### Example 4

Based on the results of the analysis of the above peptides, a new set of peptides were prepared, as designated in FIG. 12 with an asterisk. To identify the specific combination of OP-1 structural elements that are required to effect signal transduction, an initial set of contiguous peptides that combine structural elements from different regions of the molecule were designed. A similar approach has been used to develop an effective peptide mimetic of the ˜18.4 Kd erythropoietin molecule.

The roles of various side chains are ascertained by synthesizing a series of peptides that substitute, one by one, each residue in the lead peptide with an alanine (alanine scan). Conformational constraints are then investigated through a combination of molecular modeling and synthesis of variations on lead peptides in which both the placement and handedness (L-versus D amino acid) of the cyclizing cysteines are varied. Both of these structural studies and peptide studies are parallel and complementary paths toward identification of the structural features and conformational constraints of ideal OP-1 agonists and antagonists. As 3DS structural information becomes available, it is incorporated into the design of peptides.

Finger 1 peptides: Two points in the large omega loop of finger 1 are close enough to be bridged by a disulfide bond thereby making possible the design of peptide F1-3 (SEQ ID No. 7), a smaller cyclized loop than F1-2. An initial Glu is added because modeling has shown that its carboxylic acid group is able to superimpose with that of Glu⁶⁰ which is not included in F1-3, but which may be important for activity. Peptide F1-4 (SEQ ID No. 9) corrects an error in the design of F1-2 by shifting the N-terminal cyclizing cysteine to the position of Phe47. Phe47 and Glu60 define the beginning and end, respectively, of the finger 1 omega loop. Shifting the end points of the peptide loop changes the distribution of conformations accessible to it and thus may result in a change in binding affinity, relative to F1-2.

Heel peptides: Peptide H-3C (SEQ ID No. 14) is a simplified version of H-2C. Double disulfide bonds are included in H-2C in order to enforce a helical conformation. H-3C incorporates a single disulfide bond to produce a more flexible peptide that will be a more homogeneous preparation with which to further study the importance of this region on OP-1 binding and activity. Furthermore, by incorporating a D-cysteine at one or the other cyclization points we can change the distribution of conformations of the loop and thus probe the conformational requirements for tight binding.

Finger 2 peptides: Peptide F2-3 covers the tip of finger 2 plus 4 residues C-terminal to the cyclization point. An alanine scan on F2-3 identifies which residue side chains are critical for receptor binding and activity. In addition, peptides F2-4, F2-5, and F2-6 (SEQ ID Nos. 21-23, respectively) employ variations on the endpoint positions of the cyclizing disulfide bond to gain information on the conformational constraints of the receptor-bound species. Again, by incorporating a D-cysteine at one or the other cyclization points, the distribution of conformations of the loop in these peptides can be altered, permitting further probing of the conformational requirements for tight binding.

Contiguous peptides: Signal transduction in the TGFβ/BMP pathway is known to require the bringing together of a type I and a type II receptor. Our peptide studies provide a unique opportunity to investigate which structural elements of OP-1 are required to facilitate the process. Based on the ROS cell responsiveness data from phase I (FIG. 12), the F2-3 peptide is clearly able to affect part of the initial signaling process. One hypothesis is that a divalent entity is required for signaling, hence the two-fold symmetry of the OP-1 dimer. To this end we have designed peptides F2-D1 and F2-D2 (SEQ ID Nos. 24 and 25, respectively). F2-D1 is a dimer of F2-3, with the C-terminal tail extended to achieve the same finger 2 tip spacing as in OP-1 by linking the C-termini through a third disulfide bond. Because oxidizing a third disulfide may prove problematic we designed F2-D2 which is simultaneously polymerized off of both the N-terminal amino group and the ε-amino group of lysine, thereby creating a tail-to-tail dimer without the additional oxidation step.

A second hypothesis is that OP-1 acts as an adaptive interface between the type I and type II receptors and that, while the finger 2 region of OP-1 is important, it is not sufficient to initiate this process. To this end, we designed F1F2-1 (SEQ ID No. 26) to see whether joining physically proximal, but sequentially distant, regions of OP-1 is sufficient to bring the two receptors together. F1F2-1 is difficult to oxidize properly due to the adjacent cysteines in the middle, so purity may be a problem. From the known tertiary structure data it is apparent that two alanines can be added between the cysteines without loss of structural geometry, hence the design of F1F2-2 (SEQ ID No. 27). Peptide F2-10 (SEQ ID No. 28) is designed to explore the possible significance Glu60 in the finger 1 region by extending the N-terminus of F2-3 with two alanines and a glutamic acid so as to emulate Glu60.

Control peptides: Control peptides in FIG. 12 are designated with a suffix of “c”. The control peptides are simply the reverse of the sequence of the primary peptide cyclized between the same corresponding positions.

Synthesis of Peptides: Peptides are synthesized by a standard Fmoc solid-phase method using the PS-3 automated peptide synthesizer (Rainin, Woburn, Ma). Peptides having no more than one disulfide bridge are cleaved from the resin using standard cleavage and HPLC purification procedures. Peptides containing one disulfide bridge are synthesized using the trityl (Trt) derivative of Fmoc-Cys. The SH groups are fully deprotected during the standard cleavage procedure, are oxidized in solution using K₃Fe(CN)₆, and subsequently purified. Peptides containing two internal disulfide bridges are synthesized using Trt and acetamidomethyl (Acm) derivatives of cysteine. Cyclization of disulfides is carried out by a two-stage oxidation of the resin-bound peptide, first the Trt-protected residues using iodine and then the Acm protected residues as described by Kamber et al. Cyclized peptide is then cleaved from the resin using standard cleavage conditions excluding ethane dithiol. When necessary, cyclization of peptides can be carried out by the introduction of an isopeptide bond between the e-aminogroup of lysine and the carboxy-terminal carboxy group of the peptide. In this case, peptides are synthesized on acid-labile resin with the e-aminogroup of the relevant lysine protected by a 1-4,4-dimethyl-2,6-dioxocyclohexylidineethyl (Dde) group. The Dde group can be removed from the resin-bound peptide by treating with 2% hydrazine in dimethylformanmide. Finally, the peptide is cleaved from the resin, purified, and then cyclized in solution using PyBOP as an activation reagent and in the presence of hydroxybenzotriazole and N-methylmorpholine. Purified peptides are analyzed by both N-terminal amino acid sequencing and amino acid analysis.

OP-1 Binding Assays

i. Radio-receptor Assay: The receptor-binding activities of various OP-1 synthetic peptides can be determined by an equilibrium displacement binding isotherm assay using ¹²⁵I-labeled OP-1 as ligand and OP-1 receptor-enriched plasma membrane fraction of ROS cells. Highly purified OP-1 can be radioiodinated to a specific activity of 70-78 μCi/ug by a modified procedure of lactoperoxidase method. The percent bindability of radioiodinated OP-1 to excess receptor is 30-37%. Plasma membranes enriched with OP-1 receptors from ROS cells can be isolated as is known in the art. These preparations contain a single class of OP-1 binding sites with an affinity (Ka) of 4.38×10⁹ M⁻¹ and an average OP-1 binding capacity of 3.6 pmol/mg protein. In a typical assay, fixed amounts of ¹²⁵I-labeled sol. OP-1 (˜80,000 cpm) are incubated with fixed amount of receptor-enriched plasma membrane fraction in the presence (5 μg, for non-specific binding) or absence of excess unlabeled OP-1 (for total binding). Displacement curves are generated with increasing concentrations of OP-1 peptide (0.032-50 μM) or OP-1 standard preparation (5-2000 ng). The assay incubations are carried out in a reaction volume of 300 μl with shaking for 22 hrs. at 4° C. Separation of bound and free ¹²⁵I-labeled OP-1 is by centrifugation (39,500×g, 30 minutes). The supernatants are aspirated and pellets are washed before counting in a gamma counter (Packard Instr., Downers Grove, Ill.). The concentration of OP-1 peptide required to give 50% inhibition of total specific binding of ¹²⁵I-labeled OP-1 (ED₅₀) is calculated from competitive data.

ii. Type I receptor-based ligand binding method: This method uses ¹²⁵I-labeled OP-1 as ligand and ALK-6 as type I receptor for OP-1. Briefly, COS-1 cells are transfected with pSV7d vector containing ALK-6 cDNA (type I receptor for OP-1) After two days, the cells are washed with binding buffer (phosphate-buffered saline containing 0.9 mM CaCl₂, 0.49 mM MgCl₂ and 1 mg/ml BSA) and incubated on ice in the same buffer with ¹²⁵I-labeled OP-1 in the presence or absence of excess unlabeled OP-1 or test peptide. Cells are washed, and cross-linking is done in the binding buffer without BSA together with 0.14 mM of disuccinimidyl suberate (Pierce Chemical Co) for 15 min. on ice. The cells are harvested by the addition of 1 ml of detachment buffer (10 mM Tris-HCl, pH 7.4, 1 mM EDTA, 1% Triton X-100, 0.3 mM PMSF, 1% aprotinine) and incubated for 40 min on ice. The cells are pelleted by centrifugation, then resuspended in 50 μl of solubilization buffer (10 mM Tris-HCl, pH 7.4 containing 125 mM NaCl, 1 mM EDTA, 1% Triton X-100, 0.3 mM PMSF, 1% aprotinin) and incubated for 40 min on ice. After centrifugation, the lysates are pre-cleared with protein A-Sepharose for 30 min at 4° C., and precipitated overnight with a polyclonal anti ALK-6 antibody. The antibody was raised against synthetic peptide corresponding to the intracellular juxta membrane part, and was kindly provided by Dr. Peter ten Dijke from Ludwig Institute for Cancer Research, Uppsala. The incubation is continued for 1.5 h after adding protein A-Sepharose. The Sepharose particles are washed three times with 50 mM Tris-HCl, pH 8.0 containing 150 mM NaCl, 1% Nonidet P-40, 0.5% sodium deoxycholate, 0.1% SDS) and the immune complexes are eluted with Laemmli SDS-sample buffer and by boiling for 5 min. Samples are analyzed by SDS-polyacrylamide gel electrophoresis in 5-12% polyacrylamide gels, and visualized by autoradiography. The binding of test peptide to type I receptor (ALK 6) is determined based on its ability to compete with ¹²⁵I-labeled OP-1 for binding to ALK-6 protein.

iii. Type H Receptor-based rapid solid phase assay: OP-1 binding activity, and that of various synthetic peptides, may be determined by a rapid solid-phase assay using 125I-labeled OP-1 as ligand and highly purified ECD of DAF-4 as type II receptor. In a typical assay, ˜1 μg of ECD of DAF-4 receptor (R) is dissolved in 20 μl of 50 mM PBSA, pH 7.4 and immobilized on PVDF membranes (Immobilon-P) by using a slot-blot apparatus (VacuSlot-VS). A similarly immobilized polyclonal anti-OP-1 antibody (diluted Ab) has served as positive control, while bovine serum albumin (globulin free) and unrelated receptor ECD have served as negative controls in this assay. The receptor-containing membranes are incubated in 50 mM HEPES buffer, pH 7.4 containing 3% bovine serum albumin (BSA) for 14 h, in cold (4° C.), to block excess protein binding sites on the membrane. The blocking buffer is then replaced with 50 mM HEPES, pH 7.4 containing 0.5% BSA, 10 mM MgCl₂ and 1 mM CaCl₂, and the receptor-containing membranes are further incubated with ¹²⁵I-labeled OP-1 (˜400,000 cpm/ml) in the presence or absence of excess unlabeled OP-1 or test peptide for 18 h, with slow shaking at room temperature. Finally, the membranes are washed four times with 50 mM HEPES buffer, pH 7.4 containing 5 mM MgCl₂ and 1 mM CaCl₂, air dried and ¹²⁵I kept for autoradiography overnight.

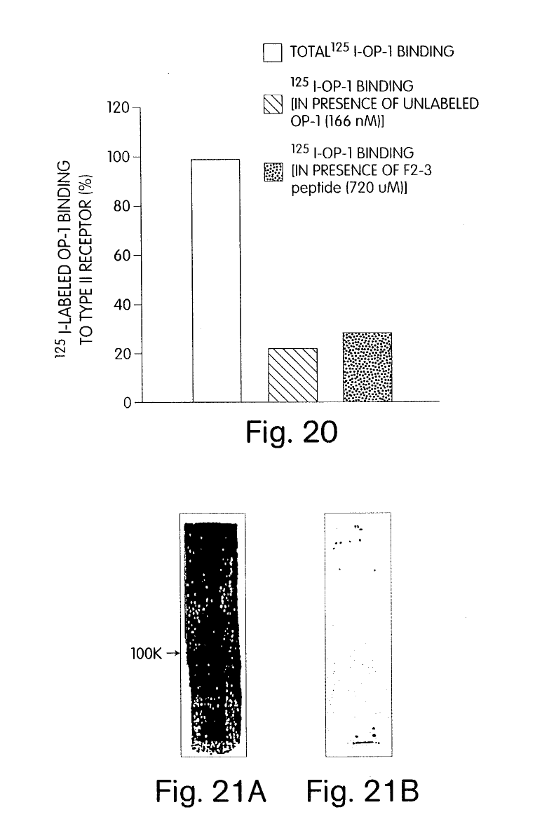

Initial binding experiments were carried out to examine the role of the finger 2 region (Leu109-Arg129) of OP-1 in the interaction of OP-1 with the type II receptor. Incubation of DAF4 with radioiodinated OP-1 was done in the presence (a) and absence (b) of synthetic peptide (F2-3) corresponding to the indicated finger 2 region of OP-1. In parallel, immobilized receptor was incubated with radioiodinated OP-1 in the presence of unlabeled OP-1 which served as a positive control. Results indicated that both unlabeled OP-1 and OP-1 peptide (F2-3) effectively inhibited (78% and 75%, respectively) ¹²⁵I-labeled OP-1 binding to the ECD of DAF-4 (FIG. 20). These initial results thus identify that the finger 2 region (Leu109-Arg129) of OP-1 is likely a contact region in the interaction of OP-1 with the type II receptor.

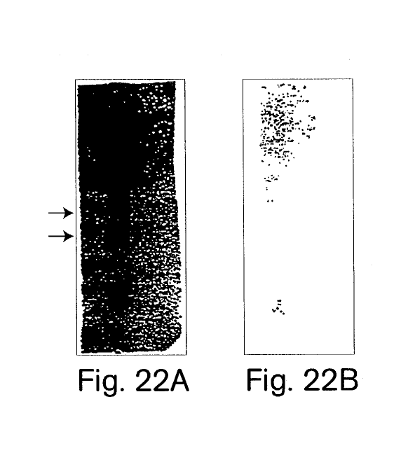

iv. Type II receptor-based ligand-blot technique: A ligand blot method can be used to characterize OP-1 binding properties of highly purified DAF4 ECD and ROS cell receptor. Peptides are analyzed by this method to determine their ability to interact with type II receptor, based on their ability to inhibit ¹²⁵I-labeled OP-1 binding to the receptor. In a typical experiment, receptor-enriched ROS cell plasma membrane fraction or purified DAF-4 ECD is treated with SDS (final concentration 1.6%, w/v) in the presence of 17% glycerol on ice, as is known in the art, and subjected to SDS-PAGE under non-reducing conditions and without prior heating of the samples, according to the procedure of Laemmli. Receptor samples are electrophoressed at 35 mA and 4° C., in gradient (5-8% or 5-10%, w/v) acrylamide separating gels. Pre-stained markers of known MW, are used as standards. After SDS-PAGE, the resolved proteins from acrylamide gels are transblotted onto PVDF membranes (Immobilon-P transfer membranes) using an Pharmacia-LKB 2005 Transphor Unit, at constant current (0.2 A) and 4° C., for 16 h. Ligand blotting is carried out as follows. Briefly, the sample lanes are incubated with blocking buffer (3% BSA in 50 mM HEPES buffer, pH 7.4) overnight in cold (4° C.), and further incubated in 50 mM HEPES buffer, pH 7.4 containing 0.5% BSA, 10 mM MgCl₂, 1 mM CaCl₂ and ¹²⁵1-labeled OP-1 (400,000 cpm/ml) in the absence or presence of excess unlabeled OP-1. Receptor 125 containing lanes are also incubated with ¹²⁵I-labeled OP-1 in the presence of test peptides to determine receptor-binding properties of the test peptides. The blots are rinsed three times with 50 mM HEPES buffer, pH 7.4 containing 5 mM MgCl₂, and 1 mM CaCl₂, air-dried and subjected to autoradiography. Typical autoradiograms showing the specific binding of ¹²⁵I-labeled OP-1 to ROS cell receptor and DAF-4 ECD are shown in FIGS. 21 and 22. The results indicate that OP-1 specifically binds to ROS cell receptor with an apparent molecular weight of 100 Kd, which is similar to the molecular size of a recently cloned type II BMP receptor. On the other hand, DAF-4 ECD showed OP-1 binding to two closely migrated receptor components in the molecular range between 45 k-60 k. This is consistent with the earlier observations on the molecular heterogeneity for DAF-4. which is possibly due to differences in the carbohydrate composition of receptor components.

In vitro Bioassays

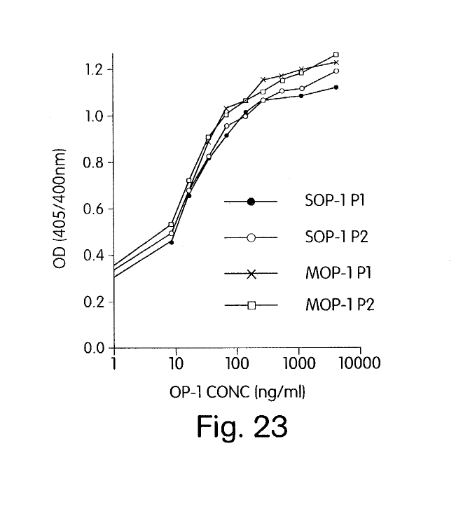

i. ROS Cell Based Alkaline Phosphatase Assay: OP-1 synthetic peptides that bind OP-1 receptors are further analyzed by in vitro bioassay. This allows defining the role of OP-1 peptides at the receptor-binding site. Some of the OP-1 peptides that effectively interact with receptors could inhibit OP-1 induced target cell responsiveness, thereby acting as functional antagonists. Alternatively, they may mimic OP-1 functions inducing alkaline phosphatase activity in ROS cells. Creative BioMolecules has developed and fully validated ROS cell in vitro assay for OP-1. An assay procedure as described earlier is essentially followed to determine the biological activities of OP-1 peptides. In a typical experiment, rat Osteosarcoma (17/2.8) cells are plated in 96 well plates (3.0×104 cells/well) and incubated overnight at 37° C. in 5-6% CO₂ incubator. Next day, the plates are examined to make sure that cells are healthy & confluent. Cells are treated with increasing concentrations of OP-1 standard (1-10,000 ng/ml) or OP-1 synthetic peptides (0.02-200 μM) alone or with OP-1 standard prepared in medium containing 1% FBS and incubated for 2 days at 37° C. in 5-6% CO₂ incubator. The cellular content of alkaline phosphatase activity is determined by the method of Reddi and Huggins. Enzyme estimations are routinely carried out in 96 well plates. Following removal of culture medium, cells are washed with pre-warmed PBS (150 μl) and further incubated in 100 ul of pre-warmed 1% Triton X-100 for 30 minutes at 37 C. Plates are centrifuged for 10 minutes at full speed, and recovered samples (each 15 μl) are assayed for enzyme activity by adding 90 ul p-nitrophenyl phosphate (Sigma) as a substrate in 0.05 M glycine-NaOH buffer, pH 9.3 and incubating for 20 minutes at 37° C. The reaction is stopped by adding 75 μl of 0.2 N NaOH/well and absorbance at 405/490 nm is measured on a Dynatech MR 700 plate reader. Results are expressed as concentration of peptide inhibitor (with antagonist activity) required to give 50% inhibition of maximum response to OP-1 standard. On the other hand, the activities of peptides that stimulate cell responsiveness (with agonist activity) are expressed relative to OP-1 standard. FIG. 23 illustrates the dose-related effects of OP-1 preparations on stimulation of alkaline phosphatase activity as determined in ROS cell based in vitro assay.

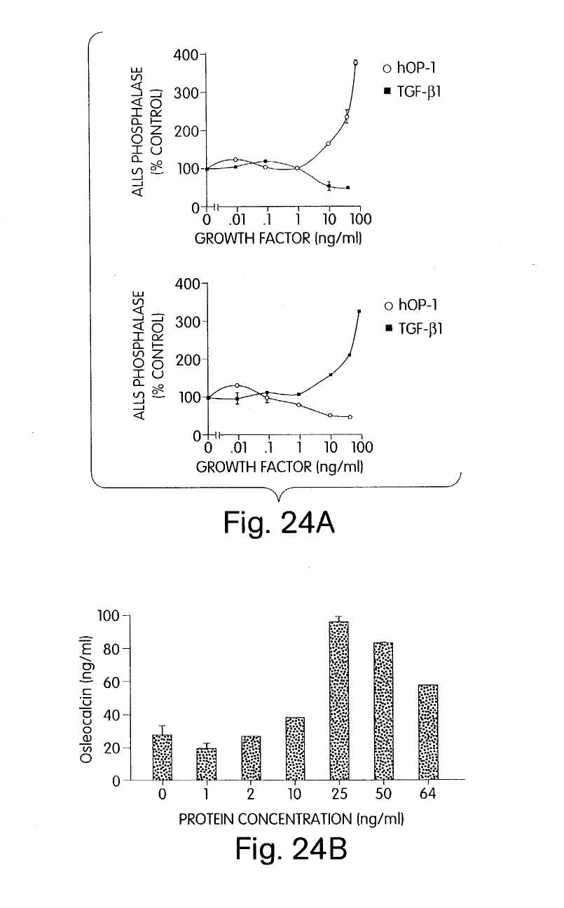

ii. Osteoblast-enriched cell culture assay: This assay is based upon the ability of OP-1 to stimulate osteoblasts in culture, and cause specific increases in phenotypic markers such as alkaline phosphatase and osteocalcin, known to be associated with the bone-forming functions of mature osteoblasts. A detailed procedure for this assay was developed in Creative and was previously published. Primary cultures of rat calvarial cells are obtained from 1-2 day-old Long-Even rats by six sequential collagenase 20-minute digests according to a modified procedure of Wong and Cohn. Single suspensions are obtained at each digest interval and numbered as individual populations 1-6 and are pooled as populations 1-2, 3-5, and 6. Confluent cultures of population 3-5 cells and population 6 cells are maintained in serum-free medium and are treated with increasing concentrations of OP-1, test peptide alone or OP-1 and test peptide for 72 h. Cells are extracted with assay buffer (0.15 M NaCl, 3 mM NaHCO₃, pH 7.4, and 0.1% Triton) and recovered samples are assayed for alkaline phosphatase activity by the method of Reddi and Huggins. The specific activity of alkaline phosphatase (units/mg of protein) is presented as the percent of serum free medium controls. For determining the effects of test peptides on osteocalcin synthesis in cultured osteoblasts, population 3-5 cells are cultivated in medium containing 10% FBS. Beginning on day 5, cells are supplemented twice a week with fresh 10 mM beta-glycerophosphate and L-(+)-ascorbate. Varying concentrations of OP-1, test peptide alone or OP-1 and test peptide are added to the cultures at the day 5 and at every feeding. Control cultures received equal volumes of buffer in which OP-1 and test peptides are made. Osteocalcin in the medium on day 13 was measured by RIA and represented as nanograms/ml culture medium. Typical responses of osteoblasts to OP-1 in terms of alkaline phosphatase activity and osteocalcin are shown in FIGS. 24A and 24B.

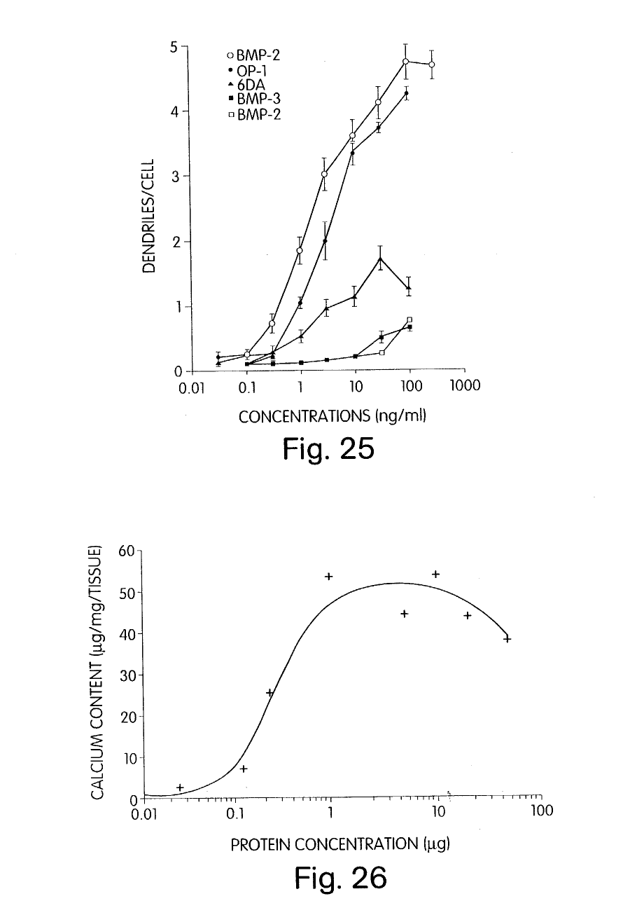

iii. Assay of dendritic growth in rat sympathetic neurons: This assay is based on the ability of OP-1 to specifically induce dendritic growth in perinatal rat sympathetic neurons. The assay has been set up in Creative as described earlier. In brief, sympathetic neurons are dissociated from the superior cervical ganglia of perinatal rats (Holtzman rat fetuses of 20-21 day pregnancy) according to the method of Higgins et al. They are then plated at low density (˜10 cells/mm²) onto polylysine-coated (100 μg/ml) coverslips and maintained in a serum-free medium containing beta-NGF (100 ng/ml). Normeuron cells are eliminated by treatment with an antimitotic agent (cytosine-beta-D-arabinofuranoside, 1 μM) on days 2 and 3. One to two days are then allowed for recovery before beginning experimental treatment. Beginning on day 5, the medium of cultures is continuously supplemented with OP-1 (50 ng/ml) or varying concentrations of test peptide or OP-1 and test peptide for 5 days and then immunostained with a dendrite specific antibody (SM132, an antibody to nonphosphorylated forms of the M and H neurofilament subunits, Sternberger Monoclonals, Inc.). Cellular morphology is analyzed by fluorescence microscopy. Results are presented as the number of dendrites per cell. Neurons in control cultures typically have only 1 process, a long axon, while neurons exposed to OP-1 are multipolar, having several tapered dendrites and 1 axon. FIG. 25 illustrates a dose-related effect of OP-1 on dendritic growth.

In viva Bioassay

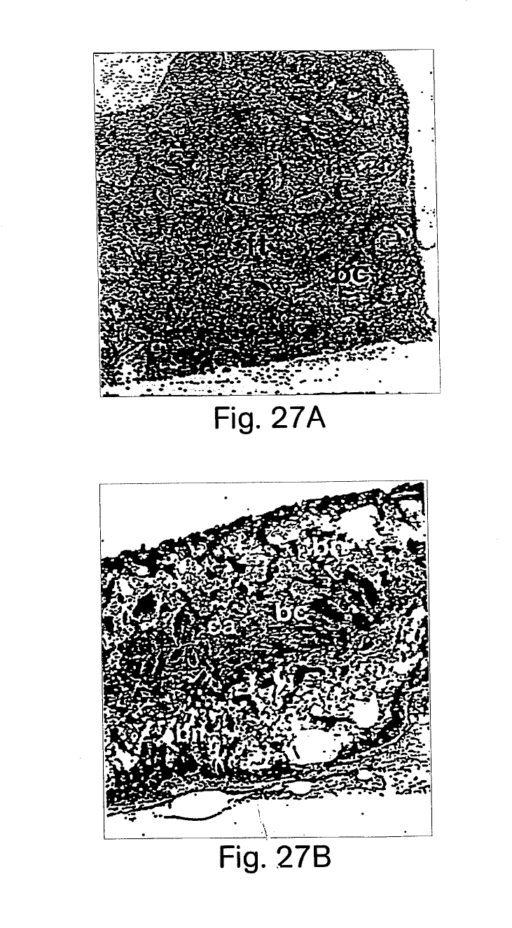

i. Subcutaneous implantation assay in rodents: This assay is based on the ability of OP-1 to induce bone formation in the rat. Creative has developed and fully validated this method. A procedure previously described in the art may be essentially followed to determine in vivo efficacy of test peptides. Briefly, 1.2 mg of bovine bone matrix (collagen carrier) is added to OP-1, test peptide alone or OP-1 and test peptide in 200 μl of 50% acetonitrile, 0.15 μl trifluoroacetic acid, mixed, and then lyophilized. Bovine bone collagen carrier alone is used as negative control. A minimum of 6 animals per group are used. The day of implantation is designated as day 0 of the assay. Implants are removed on day 14 for evaluation. Bone-forming activity in the implants is monitored by the calcium content essentially as described earlier. For histology, implants are fixed in Bouin's solution, embedded in JB4 plastic medium, cut into 1 μm sections, and stained by toluidine blue. The specific bone-forming activity is expressed as the amount of test peptide required to exhibit half-maximal bone forming activity. Photomicrographs of the implants showing OP-1 induced bone formation, have been published earlier. Characteristic response to OP-1, in terms of increase in calcium content is shown in FIG. 26. FIGS. 27A and 27B are photomicrographs of implants in rats showing negative control (bone matrix alone) and OP-1 induced bone formation by day 14.

References which may be useful in practicing the above experiments and/or other embodiments of the above-described invention include:

ten Dijke P, Miyazono K & Heldin C H (1996) Curr Opin Cell Biol 8(2), 139-145.

Hogan B L (1996) Genes Dev 10(13), 1580-1594

Dudley A T, Lyons K M & Robertson E J (1995). Genes Dev 9(22), 2795-2807

Sampath, T. K., Maliakal, J. C., Hauschka, P. V. Jones, W. K., Saak, H., Tucker, R. F., White, K. H., Coughlin, J. E., Tucker, M. M., Pang, R. H. L., Corbett, C., Ozkaynak, E., Oppermann, H., & Rueger, D. C. (1992) J. Biol. Chem. 267, 20352-20362

Cook, S. D., Baffes, G. C., Wolfe, M. W., Sampath, T. K., & Rueger, D. C. (1995) J. Bone Jt. Surg. Am. 76A, 827-838

Rutherford, R. B., Sampath, T. K., & Rueger, D. C. (1992) Int. J. Oral Maxillofac Inplants 7, 297-301

Vukicevic S, Kopp J B, Luyten F P & Sampath T K (1996). Proc Natl Acad Sci USA: 93(17), 9021-9026

Lefer A M, Tsao P S, Ma X L & Sampath T K (1992). J Mol Cell Cardiol 24(6), 585-593

Lein P, Johnson M, Guo X, Rueger D & Higgins D (1995). Neuron 15(3), 597-605

Melton, L. J. III. (1990) in Osteoporosis [eds. Chrisiansen, C. and Overgaard, K.], Third International Symposium on Osteoporosis, Copenhagen Denmark. Handelsrykeriet Aalborg ApS, Aalborg, Denmark. p.36-41.

Heldin C H, Miyazono K & ten Dijke P (1997). Nature 390(6659), 465-471.

Griffith, D. L., Oppermann, H., Rueger, D. C., Sampath, T. K., Tucker, R. F., & Carlson, W. D. (1994) J. Mol. Biol. 244, 657-658

Griffith, D. L., Keck, P., Sampath, T. K., Rueger, D. C. & Carlson, W. D. (1995) Proc. Natl. Acad. Sci. USA 93, 878-883.

Schlunnegger, M. & Grutter, M., (1992) Nature 358, 430-434

Daopin S, Li M & Davies D R (1993). Proteins 17(2), 176-192.

Lee B & Richards F M (1971). J Mol Biol 55(3), 379-400.

Shannon, C. E. & W. Weaver, The Mathematical Theory of Communication, Univ. of Illinois Press, Urbana, Ill., 1949.

Greer, J. (1991). Methods Enzymol. 202, 239-252.

Gilson M K & Honig B H (1988). Proteins 3(1), 32-52.

Gilson, M. K., Sharp, K. A. & Honig, B. H. (1987). J. Comp. Chem. 9,327-335.

Veeraraganvan, K., Jones, W., White, K., Coughlin, J., Tucker, R., Dattatreyamurty B. & Sampath T. K. (1997). Identification of osteogenic protein-1 (OP-1/BMP-7) in human blood and urine. 2^{nd} International Conference on Bone Morphogenetic Proteins, June 1997, Sacramento, Calif. Abst. 103.

Wrighton N C, Farrell F X, Chang R, Kashyap A K, Barbone F P, Mulcahy L S, Johnson D L, Barrett R W, Jolliffe L K & Dower W J (1996). Science 273(5274), 458-464

Cunningham B C, Ultsch M, De Vos A M, Mulkerrin M G, Clauser K R & Wells J A (1991). Science 254(5033), 821-825

Atherton, E. & Sheppard, R. C. (1995) In: Solid phase peptide synthesis, IRL Press,

Toth, G., Kramer, T. H., Knapp, R., Lui, G., Davis, P., Burks, T. F., Yamamura, H. I. & Hruby, V. J. (1990) J. Med. Chemistry 33, 249

Kamber, B., Hartmann, A., Eisler, K., Riniker, B., Rink, H., Sieber, P. & Rittel, W. (1980) Helv. Chim. Acta 63, 899

Hone, N. D., Chabra, S. R., Chan, W. C. & Bycroft, B. W. (1992) Poster P63, 22^{nd} European peptide Symposium, Interlaken Switzerland.

Martinez, J., Bali, J. P., Rodriguez, M., Castro, B., Laur, J. & Lignon, M. F. (1988) J. Med. Chem. 28, 1874

Schneyer, A. L., Sluss, P. M., Dattatreyamurty, B. & Reichert, L. E. Jr. (1986) Endocrinology 119, 1446-1453

Dattatreyamurty, B., Schneyer, A. L. & Reichert, L. E. Jr. (1986) J. Biol. Chem. 261, 13104-13113.

ten Dijke P, Yamashita H, Sampath T K, Reddi A H, Estevez M, Riddle D L, Ichijo H, Heldin C H & Miyazono K (1994) J. Biol Chem 269(25), 16985-16988

Dattatreyamurty, B. & Reichert Jr., L. E. (1992). Endochronology 131, 2437-2445.

Laemmli, U K (1970). Nature 227, 680-685.

Estevez M, Attisano L, Wrana J L, Albert P S, Massague J & Riddle D L (1993). Nature 1365(6447), 644-649

Maliakal, J. C., Asahina, I., Hauschka, P. V. & Sampath, T. K. (1994) Growth Factors 11, 227-234

Reddi, A. H. & Huggins, C. B. (1972) Proc. Natl. Acad. Sci. USA 69, 1601-1605.

Wong G L & Cohn D V (1975) Proc Natl Acad Sci USA 72(8), 3167-3171

Higgins, D., Lein, P., Osterhout, D. & Johnson, M. I. (1991). In Culturing Nerve Cells, G. Bonker and K. Goslin, eds. (Cambridge, Mass.; MIT Press) pp. 177-205.

Those skilled in the art will recognize, or be able to ascertain using no more than routine experimentation, many equivalents to the specific embodiments of the invention described herein. Such equivalents are intended to be encompassed by the following claims.

All patents and publications cited above are hereby incorporated by reference herein in their entirety.

We claim:

1. A peptide that antagonizes a BMP-like biological activity, wherein the peptide comprises a peptide sequence having between 6 and 50 amino acid residues including at least five contiguous amino acids of at least one of SEQ ID Nos. 1, 2, 30, 31, 34, 35, 38, 39, 42, 43, 46, 47, 50, 51, 54, 55, 58, 59, 62, and 63.

2. The peptide of claim 1, wherein the peptide sequence has between 6 and 50 amino acid residues.

3. The peptide of claim 1, wherein the peptide sequence has between 8 and 40 amino acid residues.

4. The peptide of claim 1, wherein at least 90% of the amino acid residues of the peptide sequence are contiguous amino acid residues of at least one of SEQ ID Nos. 1, 2, 30, 31, 34, 35, 38, 39, 42, 43, 46, 47, 50, 51, 54, 55, 58, 59, 62, and 63.

5. The peptide of claim 1, 2, 3, or 4, wherein the peptide sequence has at least two non-adjacent cysteine residues that are joined by a disulfide bond to form a ring.

6. The peptide of claim 5, wherein the ring comprises between 8 and 30 amino acids.

7. The peptide of claim 5, wherein the cysteine residues are located on either end of the peptide sequence, such that the ring comprises all of the amino acid residues of the peptide backbone.

8. The peptide of claim 5, wherein the peptide sequence comprises a sequence at least 90% identical to at least one of SEQ ID Nos. 5, 6, 7, 9, 11, 12, 13, and 14.

9. The peptide of claim 5, wherein the peptide sequence comprises a sequence at least 95% identical to at least one of SEQ ID Nos. 5, 6, 7, 9, 11, 12, 13, and 14.

10. The peptide of claim 8, wherein the peptide antagonizes the biological activity of OP-1.

11. A peptide having a peptide sequence comprising any one of SEQ ID Nos. 5, 6, 7, 9, 11, 12, 13, and 14, wherein at least two cysteine residues are joined by a disulfide bond to form a ring.

12. A peptide that agonizes a BMP-like biological activity, wherein the peptide comprises a peptide sequence having between 6 and 50 amino acid residues including at least five contiguous amino acids residues of at least one of SEQ ID Nos. 3, 32, 36, 40, 44, 48, 52, 56, 60, and 64.

13. The peptide of claim 12, wherein the peptide sequence has between 8 and 40 amino acid residues.

14. The peptide of claim 12, wherein the peptide sequence has between 8 and 30 amino acid residues.

15. The peptide of claim 12, wherein at least 90% of the amino acid residues of the peptide sequence are contiguous amino acid residues of at least one of SEQ ID Nos. 3, 32, 36, 40, 44, 48, 52, 56, 60, and 64.

16. The peptide of claim 12, 13, 14, or 15, wherein the peptide sequence has at least two non-adjacent cysteine residues that are joined by a disulfide bond to form a ring.

17. The peptide of claim 16, wherein the ring comprises between 8 and 30 amino acids.

18. The peptide of claim 16, wherein the cysteine residues are located on either end of the peptide sequence, such that the ring comprises all of the amino acid residues of the peptide backbone.

19. The peptide of claim 16, wherein the peptide sequence comprises a sequence at least 90% identical to at least one of SEQ ID Nos. 16, 17, 19, 21, 22, 23, 24, 25, 26, 27, and 28.

20. The peptide of claim 16, wherein the peptide sequence comprises a sequence at least 95% identical to at least one of SEQ ID Nos. 16, 17, 19, 21, 22, 23, 24, 25, 26, 27, and 28.

21. A peptide having a peptide sequence comprising any one of SEQ ID Nos. 16, 17, 19, 21, 22, 23, 24, 25, 26, 27, and 28, wherein at least two cysteine residues are joined by a disulfide bond to form a ring.

22. A peptide having a peptide sequence comprising any one of SEQ ID Nos. 16, 17, 19, 21, 22, 23, 24, 25, 26, 27, and 28, wherein at least two cysteine residues are joined by a disulfide bond to form a ring, and wherein said peptide agonizes a biological activity of OP-1.

23. A cyclic peptide that antagonizes a BMP-like biological activity, wherein the peptide comprises a peptide sequence having a cysteine residue on each end and including at least three contiguous amino acids of at least one of SEQ ID Nos. 1, 2, 30, 31, 34, 35, 38, 39, 42, 43, 46, 47, 50, 51, 54, 55, 58, 59, 62, and 63, and wherein the cysteine residues are linked together by a disulfide bond.

24. The cyclic peptide of claim 23, wherein the peptide sequence includes at least ten contiguous amino acids of at least one of SEQ ID Nos. 1, 2, 30, 31, 34, 35, 38, 39, 42, 43, 46, 47, 50, 51, 54, 55, 58, 59, 62, and 63.

25. A cyclic peptide that agonizes a BMP-like biological activity, wherein the peptide comprises an OP-1 peptide sequence bounded by a cysteine residue on each end and consisting essentially of at least three contiguous amino acids of at least one of SEQ ID Nos. 3, 32, 36, 40, 44, 48, 52, 56, 60, and 64, and wherein the cysteine residues are linked together by a disulfide bond.

26. The cyclic peptide of claim 25, wherein the peptide sequence includes at least ten contiguous amino acids of at least one of SEQ ID Nos. 3, 32, 36, 40, 44, 48, 52, 56, 60, and 64.

27. A peptide that brings together a Type I and a Type II receptor, comprising a peptide backbone having
a) a first peptide sequence having a cysteine residue on each end and including at least three contiguous amino acids of at least one of SEQ ID Nos. 1, 2, 30, 31, 34, 35, 38, 39, 42, 43, 46, 47, 50, 51, 54, 55, 58, 59, 62, and 63, and wherein the cysteine residues are linked together by a disulfide bond; and
b) a second peptide sequence having a cysteine residue on each end and including at least three contiguous amino acids of at least one of SEQ ID Nos. 3, 32, 36, 40, 44, 48, 52, 56, 60, and 64, and wherein the cysteine residues are linked together by a disulfide bond.

28. A nucleic acid sequence encoding a peptide of any preceding claim.

29. A pharmaceutical preparation comprising a sterile excipient and a peptide of claim 1, 2, 3, 5, 23, or 27.

30. A pharmaceutical preparation comprising a sterile excipient and a peptide of claim 12, 13, 14, 16, or 25.

31. A pharmaceutical preparation of claim 30, further comprising a Bone Morphogenic Protein (BMP).

32. A pharmaceutical preparation of claim 30, further comprising an agent other than the peptide, which agent promotes growth, differentiation, or proliferation of a cell.

33. A pharmaceutical preparation of claim 32, wherein the agent is a cytokine, growth factor, or morphogen.

34. A method for inhibiting growth, differentiation, or proliferation of a cell, comprising contacting the cell with a peptide of claim 1 or 21.

35. A method for promoting growth, differentiation, or proliferation of a cell, comprising contacting the cell with a peptide of claim 12 or 25.

36. The method of claim 35, further comprising contacting the cell with a BMP.

37. The method of claim 36, wherein the BMP is OP-1.

38. The method of claim 35, further comprising contacting the cell with an agent other than the peptide, which agent promotes growth, differentiation, or proliferation of a cell.

39. The method of claim 38, wherein the agent is a cytokine, growth factor, or morphogen.

40. A method for inhibiting growth, differentiation, or proliferation of a cell, comprising contacting the cell with a nucleic acid sequence encoding a peptide of claim 1 or 23.

41. A method for promoting growth, differentiation, or proliferation of a cell, comprising contacting the cell with a nucleic acid sequence encoding a peptide of claim 12 or 25.

42. The method of claim 41, further comprising contacting the cell with a BMP.

43. The method of claim 42, wherein the BMP is OP-1.

44. The method of claim 41, further comprising contacting the cell with an agent other than the peptide, which agent promotes growth, differentiation, or proliferation of a cell.

45. The method of claim 44, wherein the agent is a cytokine, growth factor, or morphogen.

46. A method of any of claims 35 to 45, wherein the cell is a bone, cartilage, nerve, or liver cell.

47. A peptidomimetic of a peptide of any of claims 1-27.

48. A peptide that antagonizes a biological activity of a morphogenic protein of the BMP/OP subfamily, wherein the peptide comprises a peptide sequence having a cysteine residue on each end and including at least three contiguous amino acids of a region corresponding to a Finger 1 or Heel region of OP-1, and wherein the cysteine residues are linked together by a disulfide bond.

49. The cyclic peptide of claim 48, wherein the peptide sequence includes at least ten contiguous amino acids of the Finger 1 or Heel region.

50. A peptide that agonizes a biological activity of a morphogenic protein of the BMP/OP subfamily, wherein the peptide comprises a peptide sequence having a cysteine residue on each end and consisting essentially of at least three contiguous amino acids of a region corresponding to a Finger 2 region of OP-1, and wherein the cysteine residues are linked together by a disulfide bond.

51. The cyclic peptide of claim 50, wherein the peptide sequence includes at least ten contiguous amino acids of SEQ ID No. 3.

52. A peptide that brings together a Type I and a Type II receptor, comprising a peptide backbone having
a) a first peptide sequence having a cysteine residue on each end and including at least three contiguous amino acids of a region corresponding to a Finger 1 or Heel region of OP-1, and wherein the cysteine residues are linked together by a disulfide bond; and
b) a second peptide sequence having a cysteine residue on each end and including at least three contiguous amino acids of a region corresponding to a Finger 2 region of OP-1, and wherein the cysteine residues are linked together by a disulfide bond.
